(19) United States
(12) Patent Application Publication
(10) Pub. No.: US 2023/0203078 A1
(43) Pub. Date: Jun. 29, 2023

(54) SYNTHESIS OF 3'N NUCLEOSIDES THROUGH OXIME INTERMEDIATES AND RELATED COMPOUNDS

(71) Applicant: JANSSEN BIOPHARMA, INC., SAN FRANCISCO, CA (US)

(72) Inventors: Sergei Gryaznov, San Mateo, CA (US); Vivek Kumar Rajwanshi, Cupertino, CA (US); Ioannis N. Houpis, Antwerp (BE)

(21) Appl. No.: 17/912,418

(22) PCT Filed: Mar. 15, 2021
(86) PCT No.: PCT/IB2021/052141

§ 371 Date: Sep. 16, 2022

Related U.S. Application Data
(60) Provisional application No. 62/990,361, filed on Mar. 16, 2020.

(51) Int. Cl. C07H 1/00; C07H 19/06; C07H 19/16; C12N 15/11; C07H 19/10; C07H 19/20

(52) U.S. Cl. CPC C07H 1/00; C07H 19/06; C07H 19/16; C12N 15/111; C07H 19/10; C07H 19/20; C12N 2310/11; C12N 2310/14; C12N 2330/30

(57) ABSTRACT

Provided herein are novel synthetic routes to amines through an oxime intermediate, e.g., 3′-N nucleosides and novel and intermediate compounds produced during these synthetic procedures.

### TECHNICAL FIELD

Provided herein includes synthesis of amines through an oxime intermediate from, e.g., a secondary alcohol. Acyclic and cyclic structures are included. For example, the synthesis of 3′-N modified nucleosides and intermediate compounds thereof are included within the disclosure.

### BACKGROUND

The following description of the background is provided simply as an aid in understanding method and process described herein and is not admitted to describe or constitute prior art to the what is provided herein.

The synthesis of amines is a useful tool for synthetic chemists. Formation of amines via reduction of an azide moiety is known, but azides can be hazardous, especially when used on a scale needed for commercial manufacture of a compound.

Modified oligonucleotide compounds have gained attention over the past few years as potential therapeutic agents for numerous indications. These oligonucleotide compounds may include one or more nucleotides that are modified, e.g., at the 2′ and/or 3′ position of the sugar moiety. However, synthetic routes to the nucleoside building blocks of these modified oligonucleotides often include multiple synthetic steps with low overall yield, purity, and/or use of reagents that are suboptimal for synthesis on a scale needed for commercial manufacture of the ultimate modified oligonucleotide compound. Thus, a need exists for new, more facile synthetic routes to modified nucleosides that can be used, e.g., in the preparation of oligonucleotide compounds.

### SUMMARY

Provided herein are novel synthetic routes to amines through an oxime intermediate, e.g., 3′-N nucleosides and novel and intermediate compounds produced during these synthetic procedures. The new synthetic routes described herein to form amine-substituted moieties such as ribose or carbocyclic moieties which can be useful for, e.g., 3′-N nucleosides or 5′ modified nucleotides and novel and intermediate compounds produced during these synthetic procedures.

Some embodiments relate to a method of producing a nucleoside of formula (III):

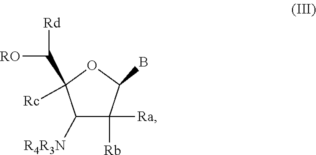

wherein B is an optionally protected nucleobase; R is H, a counterion or a protecting group, PG1; Ra and Rb are each independently selected from the group consisting of H, halogen, R¹, OR¹, OPG and OR²OR¹; Rc is selected from the group consisting of H, R¹, OPG, OR¹ and N(R₉)₂; Rd is H or R¹; R₃ is PG2 or OPG, and R₄ is H, OAc, or Ac, or R₃ and R₄ together form a cyclic protecting group, cPG, R¹ is C₁₋₃alkyl optionally substituted with one or more fluoro or PG, R² is C₁₋₅alkylene optionally substituted with one or more fluoro, each R⁹ is independently H or a C₁₋₆alkyl. In some embodiments, the method comprises preparing a 3′-oxime modified nucleoside; converting the 3′-oxime modified nucleoside to a 3′-NH modified nucleoside; and converting the 3′-NH modified nucleoside to a compound of formula (I). In some embodiments, at least one of Ra and Rb is not H.

The methods and compounds described in this application are notably useful in the field of the production of oligonucleotides, such as synthetic or modified nucleotides and antisense oligonucleotides (ASOs) or small interfering RNAs (siRNAs). The application encompasses more particularly a method of producing an antisense oligonucleotide (ASO) or a small interfering RNAs (siRNA), wherein the ASO or siRNA comprises at least one nucleoside of a formula described herein (e.g., formula (III)), wherein the method comprises producing the at least one nucleoside of a formula described herein (e.g., the formula (III)) by the method described herein.

Some embodiments relate to a method of producing a nucleoside of formula (III):

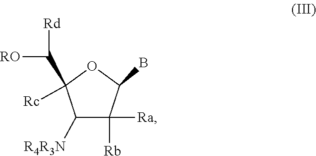

wherein B is an optionally protected nucleobase; R is H, a counterion or a protecting group, PG; Ra and Rb are each independently selected from the group consisting of H, F, R¹, OR¹, OPG and OR²OR¹; Rc is selected from the group consisting of H, R¹, OPG, OR¹ and N(R₉)₂; Rd is H or R¹; R₃ is PG or OPG, and R₄ is H, OAc, or Ac, or R₃ and R₄ together form a cyclic protecting group, cPG. R¹ is C₁₋₃alkyl optionally substituted with one or more fluoro or PG, R² is C₁₋₅alkylene optionally substituted with one or more fluoro, each R₉ is independently H or a C₁₋₆alkyl. The method comprises preparing a 3′-oxime modified nucleoside; converting the 3′-oxime modified nucleoside to a 3′-NH modified nucleoside; and converting the 3′-NH modified nucleoside to a compound of formula (I). In some embodiments, at least one of Ra and Rb is not H.

In embodiments, Rb is selected from OCF₂—CH₃, OCH₂CH₂OMe, OMe, OEt, OCH₂F, F, OTBDMS. In embodiments, the 3′-oxime modified nucleoside is represented by the following formula (I):

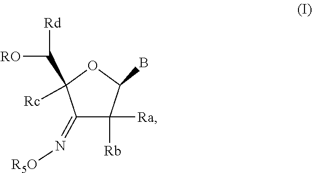

wherein B, R, Ra, Rb, Rc, Rd, P, R₁, R₂ are the same as formula (III) and R₅ is H or a C₁₋₆alkyl group (optionally substituted with an aryl group, such as phenyl). In embodiments, the 3′-NH modified nucleoside is represented by the following formula:

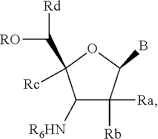

wherein B, R, Ra, Rb, Rc, Rd, P, R₁, R₂ are the same as formula (I) and R₆ is a C₁₋₃alkyl or a protecting group. In embodiments, the 3′-oxime modified nucleoside is converted to 3′-NH modified nucleoside directly through a hydroxylamine intermediate compound. In embodiments, the 3′-oxime modified nucleoside is converted to 3′-NH modified nucleoside a hydroxylamine intermediate compound in two or less steps. In embodiments, converting the 3′-oxime modified nucleoside to a 3′-NH modified nucleoside comprises a selective reduction of the 3′-oxime moiety. In embodiments, the selective reduction comprises use of NaB(OAc)₃ or pinacolborane. In embodiments, B is a protected or unprotected adenosine. In embodiments, B is a protected or unprotected guanosine. In embodiments, B is a protected or unprotected uridine. In embodiments, B is a protected or unprotected cytidine. In embodiments, the method includes one or two chromatography purification steps. In embodiments, the method does not include a chromatography purification step. In embodiments, the method is conducted on 1 kg or more 3′-oxime modified nucleoside. In embodiments, B is a protected or unprotected adenosine and Rb is F or MOE. In embodiments, adenosine is not protected with Bz. In embodiments, B is a protected or unprotected guanosine and Rb is F or MOE.

Other embodiments include a compound represented by formula (I′) or (II′):

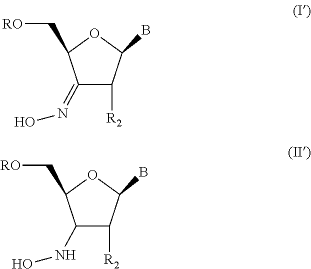

wherein B is an optionally protected nucleobase, R is H, —OH, a counterion, or a protecting group, R² is F, OR₇ or OR₈OR₇, R₇ is a C₁₋₃alkyl or fluoroalkyl, and R₈ is a C₁₋₅alkylene or fluoroalkylene.

In embodiments, R₂ is selected from OCF₂—CH₃, OCH₂CH₂OMe, OMe, OEt, OCH₂F, F, OTBDMS. In embodiments, B is a protected nucleobase. In embodiments, B is a protected adenine.

### Definitions

Unless defined otherwise, all technical and scientific terms used herein have the same meaning as is commonly understood by one of ordinary skill in the art. In the event that there are a plurality of definitions for a term herein, those in this section prevail unless stated otherwise.

As described herein, the terms “optionally substituted” and “substituted or unsubstituted” are synonyms. Whenever a group is described as being substituted, optionally or otherwise, by various indicated substituents, the group may be substituted with one or more of the indicated substituents.

As used herein, “alkyl” refers to a fully saturated linear, branched, or cyclic hydrocarbon group. The alkyl group may be a lower alkyl, having 1 to 6 carbon atoms. The alkyl group may be designated as “C1 to C6 alkyl” or similar designations, indicating that the alkyl group is a linear or branched alkyl group having up to six carbon atoms, such as methyl, ethyl, propyl, isopropyl, butyl, isobutyl, tertiary butyl, pentyl and hexyl. The alkyl group may be substituted or unsubstituted.

As used herein, “alkenyl” refers to a linear, branched, or cyclic hydrocarbon group having one or more double bonds. The double bond may be at any position, unless otherwise indicated. An alkenyl group may be unsubstituted or substituted.

As used herein, “alkynyl” refers to a linear or branched hydrocarbon group having one or more triple bonds. The triple bond may be at any position, unless otherwise indicated. An alkynyl group may be unsubstituted or substituted.

Unless otherwise indicated, “hydrocarbyl” refers to an alkyl, alkenyl, or alkynyl group.

As used herein, “aryl” refers to a monocyclic or bicyclic aromatic ring system having carbocyclic rings, unless otherwise indicated. Examples of aryl groups include, but are not limited to, benzene and naphthalene. An aryl group may be substituted or unsubstituted.

As used herein, “heteroaromatic” and “heteroaryl” refer to a monocyclic, bicyclic or tricyclic aromatic ring system that contain(s) one or more heteroatoms, including but not limited to, nitrogen, oxygen and sulfur. Furthermore, the term “heteroaromatic” and “heteroaryl” include fused ring systems where two rings, such as at least one aryl ring and at least one heteroaryl ring, or at least two heteroaryl rings, share a chemical bond. Examples of heteroaryl rings include, but are not limited to, a pyrrole ring, an imidazole ring; a pyrazole ring, an indole ring system, a benzimidazole ring system, an indazole ring system, or a purine ring system. A heteroaryl group may be substituted or unsubstituted.

As used herein, “arylalkyl” refers to an aryl group connected, as a substituent, to a lower alkylene group. The lower alkylene and aryl group of an aryl(alkyl) may be substituted or unsubstituted. Examples include but are not limited to benzyl, 2-phenyl(alkyl), 3-phenyl(alkyl), diphenylmethyl, and triphenylmethyl.

As used herein, “acyl” refers to an alkyl, alkenyl, alkynyl, or aryl group connected, as a substituent, to a carbonyl group. Examples include acetyl, propanoyl, and benzoyl. An acyl may be substituted or unsubstituted.

A “sulfonyl” group refers to an —SO₂R group, in which R can be alkyl, alkenyl, alkynyl, or aryl, heteroaryl. A sulfonyl may be substituted or unsubstituted.

The term “ester,” as used herein, refers to a —OCOR or —OSO₂R group in, which R can be alkyl, alkenyl, alkynyl, aryl, heteroaryl, or aryl(alkyl). An ester may be substituted or unsubstituted.

The term “nucleoside” is used herein refers to a compound composed of an optionally substituted ribose or deoxyribose moiety attached to a heterocyclic base via a N-glycosidic bond, such as attached via the 9-position of a purine base or the 1-position of a pyrimidine base. In some instances, the nucleoside can be a nucleoside analog.

As used herein, the term “heterocyclic base” refers to an optionally substituted nitrogen-containing heterocyclic ring compound that can be attached to a ribose or deoxyribose moiety. In some embodiments, the heterocyclic base can be selected from an optionally substituted purine base or an optionally substituted pyrimidine base. A non-limiting list of optionally substituted purine bases includes purine, adenine, guanine, hypoxanthine, xanthine, alloxanthine, theobromine, caffeine, uric acid and isoguanine. A non-limiting list of optionally substituted pyrimidine bases includes cytosine, thymine, uracil, and 5,6-dihydrouracil. Where a heterocyclic base has a ring carbonyl, an exocyclic amino substituent, or other functional groups, these groups may be protected with a protecting group by methods known in the art.

The term “protecting group” as used herein refer to any atom or group of atoms that is added to a molecule in order to prevent existing groups in the molecule from undergoing unwanted chemical reactions. Examples of protecting group moieties are described in T. W. Greene and P. G. M. Wuts, Protective Groups in Organic Synthesis, 3. Ed, John Wiley & Sons, 1999, incorporated by reference for the limited purpose of disclosing suitable protecting groups. A non-limiting list of protecting groups includes: Hydroxy protecting groups, such as methoxymethyl, ethoxymethyl, tetrahydropyran-2-yl, tetrahydrofuran-2-yl, t-butyl, allyl, benzyl, trimethylsilyl, t-butyldimethylsilyl, t-butyldiphenylsilyl, acetyl, pivaloyl, and benzoyl; 1,2-Diol protecting groups, such as acetonide and benzylidene; and Amino protecting groups, such as 9-fluorenylmethoxycarbonyl (Fmoc), t-butoxycarbonyl (Boc), benzyloxycarbonyl, phthalimide, benzyl, triphenylmethyl, and benzylidene.

The term “protected hydroxy group” as used herein refers to a moiety derived from a hydroxy group by replacing the hydroxyl hydrogen with a hydroxy protecting group. The term “protected amino group” as used herein refers to a moiety derived from an amino group by replacing at least one amino hydrogen with an amino protecting group.

As used herein, the term “counterion” refers to a positively charged ion that associates with one compound of the present invention when one of its components has a negative charge (ie, O⁻ or COO⁻). Examples of the counterions include but are not limited to H⁺, H₃O⁺, ammonium, potassium, calcium, lithium, magnesium and sodium.

Unless otherwise indicated, a person of ordinary skill in the art would understand that protecting groups can be replaced with other protecting groups which serve a similar protective function. For example, in the protection of a hydroxy group, methoxymethyl may be replaced with tetrahydropyran-2-yl, allyl, or benzoyl. In the protection of an amino group, t-butoxycarbonyl may be replaced with phthalimide, benzyl, or triphenylmethyl. Diols may be individually protected with separate hydroxy protecting groups, or protected as a cyclic acetal or ketal, e.g., as an acetonide.

Unless indicated otherwise, IUPAC numbering will be used herein. When referring to a compound of formula 1 or a derivative thereof, the ribose ring will be numbered as a tetrahydrofuran derivative. Thus, the R² group is normally identified as attached to the carbon atom in the 2-position, and fluorine is attached to the carbon atom in the 5-position, marked with an asterisk, although the numbering about the ribose ring may be reversed in some chemical names. In some cases, a compound of formula 1 or a derivative thereof may be named as a nucleoside derivative, e.g., 2′-ethynyl-4′-fluoroadenosine, where R² is adenine and R¹ is ethynyl. When a compound of formula 1 is named as a nucleoside derivative, the R² group is attached to the carbon atom in the 1′-position, and fluorine is attached to the carbon atom in the 4′-position, marked with an asterisk. Both numbering systems are known in the art, and should be understood as synonymous.

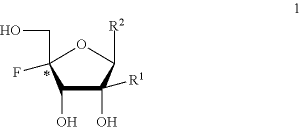

Terms and phrases used in this application and variations thereof, especially in the appended claims, unless otherwise expressly stated, should be construed as open ended as opposed to limiting. As examples of any of the foregoing, the terms “including,” “containing,” and “comprising” are synonymous, and should be read to mean “including but not limited to” or “including at least,” and do not exclude additional, unrecited elements or method steps. The term “example” is used to provide exemplary instances of the item in discussion, not an exhaustive or limiting list thereof. When used in the context of a process, the term “comprising” means that the process includes at least the recited steps, but may include additional steps. When used in the context of a compound or composition, the term “comprising” means that the compound or composition includes at least the recited features or components, but may also include additional features or components. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but should be read as “and/or” unless expressly stated otherwise.

Where a range of values is provided, it is understood that the upper and lower limit is encompassed within the range.

### DETAILED DESCRIPTION

The synthesis methods described herein help avoid the use of potentially hazardous azide and achieve a more efficient and safer preparation of the desired oligonucleotide compound. The formation of oxime intermediate during the synthesis steps as described herein simplifies the synthesis steps and reduce the manufacture cost. The methods described herein is more reliable and amenable to scale-up reactions and provides an efficient and safe option for oligonucleotide production.

### Synthetic Routes

Provided herein are methods of synthesizing amine moieties through an oxime intermediate. Oxime moieties, as discussed herein may have the following structure:

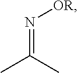

where R can be, e.g., an H or alkyl.

More particularly, am embodiment is related to a method comprising one or more of the steps in the following Scheme A.

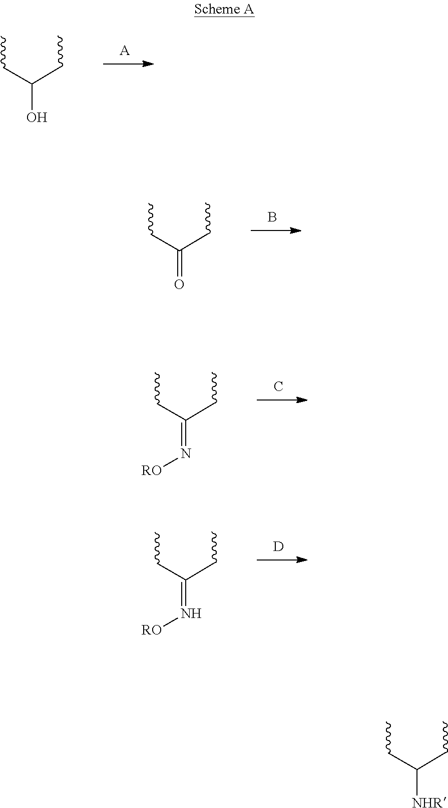

### Step A

The methods include, for example, providing a starting material having a hydroxyl or carbonyl moiety. In some embodiments, the starting material comprises a hydroxyl, which can be converted to a carbonyl via Step A. Step A may be carried out by synthetic methods disclosed in the art, e.g., an oxidation reaction. In some embodiments, the oxidation is performed using 1-ethyl-3-(3-dimethylaminopropyl)carbodiimide. Other oxidation conditions are also within the scope of this disclosure, non-limiting examples of which include Dess-Martin Oxidation, Jones Oxidation, Corey-Kim Oxidation, and Swern Oxidation. Further embodiments of this oxidation procedure are disclosed herein.

The disclosed method includes, in some embodiments, forming a 3′-oxime modified nucleoside from a starting material having an hydroxyl or carbonyl moiety is a cyclic compound, for example a ribose-type sugar or nucleoside. For example, in some embodiments, the starting material may be:

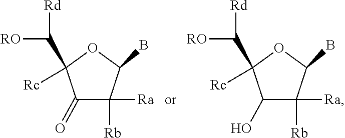

where B is an optionally protected nucleobase; R is H, a counterion or a protecting group, PG; Ra and Rb are each independently selected from the group consisting of H, F, R¹, OR¹, OPG and OR²OR¹; Rc is selected from the group consisting of H, R¹, OPG, OR¹ and N(R₉)₂; Rd is H or R¹; R¹ is C₁₋₃alkyl optionally substituted with one or more fluoro or PG; and each R₉ is independently H or C₁₋₆alkyl. In some embodiments, at least one of Ra and Rb is not H.

### Step B

A carbonyl-containing compound can be converted to an oxime moiety via Step B. The carbonyl-containing compound may be an isolated compound, or it may be carried over crude or partially purified from a previous reaction, such as the reaction in Step A. Step B may be carried out by synthetic methods suitable in the art. In some embodiments, Step B comprises a condensation of the ketone with hydroxylamine or alkylhydroxylamine. The present disclosure includes R groups other than H and C₁₋₆alkyl, as would be understood in the art, and thus, a hydroxylamine moiety used for the condensation with the ketone is not limited to the embodiments listed above. Further embodiments of this oxime conversion procedure are disclosed herein.

For example, in some embodiments, the oxime intermediate can be represented by the following formula (I):

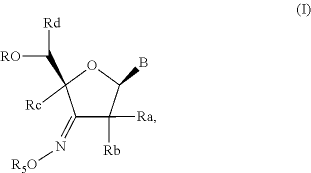

wherein
B is an optionally protected nucleobase; R is H, a counterion or a protecting group, PG; Ra and Rb are each independently selected from the group consisting of H, F, R¹, OR¹, OPG and OR²OR¹; Rc is selected from the group consisting of H, R¹, OPG, OR¹ and N(R₉)₂; Rd is H or R¹; R₅ is H or a C₁₋₆alkyl group (optionally substituted with an aryl group, such as phenyl) and R₉ is independently H or a C₁₋₆alkyl. In some embodiments, at least one of Ra and Rb is not H.

In some embodiments, R is a protecting group, such as a silyl protecting group. In some embodiments, Ra is not OH or OP. In some embodiments, Rb is H. In some embodiments, Rc is H. In some embodiments, Rd is H. In some embodiments, R⁵ is H. In some embodiments, the variables in compounds of formula (II) can be the same as embodiments for compounds of Formula (I).

In some embodiments, the 3′-oxime modified nucleoside is represented by the following formula (I′):

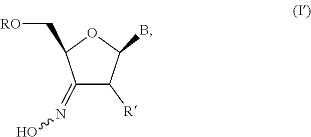

wherein B is a nucleobase, R is H, a counterion, or a protecting group, R′ F, OR¹ or OR²OR¹, R¹ is a C₁₋₃alkyl or fluoroalkyl, and R² is a C₁₋₅alkylene or fluoroalkylene.

### Step C

The oxime-containing compound can be converted to a reduced oxyamine compound, e.g., a hydroxylamine or alkoxyamine compound via Step C. The oxime-containing compound may be an isolated compound, or it may be carried over crude or partially purified from a previous reaction, such as the reaction in Step B. In some embodiments, the oxime-containing compound is reduced using, e.g., reagents known in the art to carry out the reduction, such as boranes including pinacolborane, borohydrides, and OAc-borohydrides such as NaBH(OAc)₃. Further embodiments of this reduction procedure are disclosed herein.

Reduction of the oxime moiety can be performed selectively. In some embodiments, the selective reduction comprises use of NaB(OAc)₃ or pinacolborane. The selective reduction may be performed by adding a reducing agent (e.g., OAc-borohydride or borane) at a reduced temperature, e.g., less than 10, 0, −10, −20, −30, −40, −50, −60, −70, or −80° C., or at any value within this range. In an embodiment, a OAc-borohydride or borane is added at a temperature of about −40° C. The selective reduction is allowed to occur for a certain amount of time, such as 30 min, 1, 2, 3, 4, 5, 6, 7, 8 or more hours, or at any value within this range. In an embodiment, the selective reduction is allowed to occur for a period of about 4 hours.

For example, in some embodiments, the reduced oxyamine intermediate can be represented by the following formula (II):

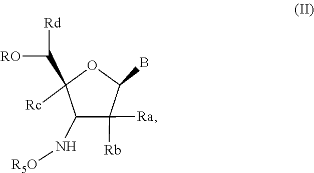

wherein
B is an optionally protected nucleobase; R is H, a counterion or a protecting group, PG; Ra and Rb are each independently selected from the group consisting of H, F, R¹, OR¹, OPG and OR²OR¹; Rc is selected from the group consisting of H, R¹, OPG, OR¹ and N(R₉)₂; Rd is H or R¹; R₅ is H or a C₁₋₆alkyl group (optionally substituted with an aryl group, such as phenyl) and R₉ is independently H or a C₁₋₆alkyl. In some embodiments, the, when R is a protecting group, this group is removed prior to reduction of the oxime moiety. In some embodiments, at least one of Ra and Rb is not H.

### Step D

The reduced oxyamine compound may be converted to an amine compound via Step D. The oxyamine-containing compound may be an isolated compound, or it may be carried over crude or partially purified from a previous reaction, such as the reaction in Step C. The reduced oxime moiety can be directly converted to a primary amine or can be converted in two steps or less. In some embodiments, Step D comprises hydrolysis of the oxyamine moiety. For example, reagents known in the art to carry out the conversion may be used, such as Pd/C and hydrogen. Further embodiments of this hydrogenation procedure are disclosed herein.

For example, in some embodiments, the resulting amine compound can be represented by formula (III):

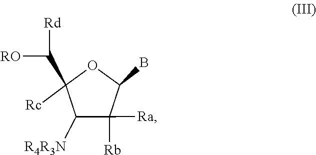

wherein

B is an optionally protected nucleobase;

R is H, a counterion or a protecting group, PG;

Ra and Rb are each independently selected from the group consisting of H, F, R¹, OR¹, OPG and OR²OR¹;

Rc is selected from the group consisting of H, R¹, OPG, OR¹ and N(R₉)₂;

Rd selected from the group consisting of H and R¹,

R³ is PG or OPG, and

R⁴ is H, OAc, or Ac, or

R³ and R⁴ together form a protecting group, such as a cyclic protecting group cPG, wherein

R¹ is C₁₋₃alkyl optionally substituted with one or more fluoro or PG,

R² is C₁₋₅alkylene optionally substituted with one or more fluoro,

each R₉ is independently selected from the group consisting of H and a C₁₋₆alkyl.

In some embodiments, at least one of Ra and Rb is not H.

Some embodiments relate to a method of producing a nucleoside of formula (III), said method comprising: preparing a 3′-oxime modified nucleoside; converting the 3′-oxime modified nucleoside to a 3′-NH modified nucleoside; and converting the 3′-NH modified nucleoside to a compound of formula (I). In some embodiments, Rb is selected from OCF₂—CH₃, OCH₂CH₂OMe, OMe, OEt, OCH₂F, F, OTBDMS. In some embodiments, the 3′-oxime modified nucleoside is represented by formula (II).

In embodiments, the 3′-NH modified nucleoside is represented by the following formula:

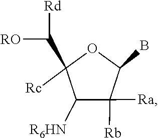

wherein
B, R, Ra, Rb, Rc, Rd, P, R₁, R₂ are the same as formula (III) and
R₆ is a C₁₋₃alkyl or a protecting group.

As used herein, the 1′ to 5′ positions refer to the traditional numbering convention for nucleotides, which is demonstrated in the following:

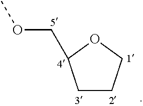

In an embodiment, PG is selected from the group consisting of a silyl protecting group, isobutyryl, Ac, Bn, Boc, TFA, CBz, Tr and MMTr. In an embodiment, R³ and R⁴ together form a protecting group, for example a benzylideneamine or cPG. In some embodiments, cPG is selected from the group consisting of phthalimide and pyrrolidinediones. Other known protecting groups are also included, such as those in T. W. Green, P. G. M. Wuts, Protective Groups in Organic Synthesis, Wiley-Interscience, New York, 1999, 503-507, 736-739.

In some embodiments, Ra is fluoro, OR¹ or OR²OR¹ and R¹ and R² are C₁₋₃alkyl optionally substituted with one or more fluoro or C₁₋₃ fluoroalkyl. OR¹ includes, for example, —OCH₃ (or OMe), —OCFH₂, —OCHF₂, OCF₃, —OCH₂OCH₃, —OCFH₂OCH₃, —OCHF₂OCH₃, —OCF₃OCH₃, —OCH₂OCFH₂, —OCH₂OCHF₂, —OCH₂OCF₃, —OCFH₂OCH₃, —OCFH₂OCFH₂, —OCFH₂OCHF₂, —OCFH₂OCF₃, —OCHF₂OCH₃, —OCHF₂OCFH₂, —OCHF₂OCHF₂, —OCHF₂OCF₃, —O(CR′₂)₃OCR′₃, —OCH₂CH₃ (or Et), —OCFH₂CH₃, —OCHF₂CH₃, —OCF₃CH₃, —OCH₂CFH₂, —OCH₂CHF₂, —OCH₂CF₃, —OCFH₂CH₃, —OCFH₂CFH₂, —OCFH₂CHF₂, —OCFH₂CF₃, —OCHF₂CH₃, —OCHF₂CFH₂, —OCHF₂CHF₂, —OCHF₂CF₃, —OCH₂CH₂CH₃, OCF₂CH₂CH₃, OCH₂CF₂CH₃, OCH₂CH₂CF₃, OCF₂CF₂CH₃, OCH₂CF₂CF₃, OCF₂CH₂CF₃, OCF₂CF₂CF₃, OCHFCH₂CH₃, OCHFCHFOCH₃, OCHFCH₂CFH₂, OCHFCH₂CHF₂ and OCH₂CHFCH₃. OR²OR¹ includes, for example, —OCH₂CH₂OCH₃ (or MOE), —OCF₂CH₂OCH₃, —OCH₂CF₂OCH₃, —OCH₂CH₂OCF₃, —OCF₂CF₂OCH₃, —OCH₂CF₂OCF₃, —OCF₂CH₂OCF₃, —OCF₂CF₂OCF₃, —OCHFCH₂OCH₃, —OCHFCHFOCH₃, OCHFCH₂OCFH₂, —OCHFCH₂OCHF₂ and —OCH₂CHFOCH₃.

In some embodiments, Ra is not OH or O-PG.

In some embodiments, at least one of Rb, Rc and Rd is H. In some embodiments, each of Rb, Rc and Rd is H.

The nucleobase may include adenine (A), guanine (G), thymine (T), cytosine (C), uracil (U), 5-methylcytosine (5meC), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 3′-amino-2′-deoxy-2,6-diaminopurine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluorometliyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine, 7-deazaguanosine, 2-aminopyridine and 2-pyridone.

The nucleobase may be a tricyclic pyrimidine such as phenoxazine cytidine(1H-pyrimido[5,4-b][1,4]benzoxazin-2(3H)-one) or phenothiazine cytidine (1H-pyrimido[5,4-b][1,4]benzothiazin-2(3H)-one), or a G-clamp such as a substituted phenoxazine cytidine (e.g., 9-(2-am-oe1hoxy)-H-pyrimido[5,4-b][1,4]benzoxazin-2(3H)-one), carbazole cytidine (2H-pyrimido[4,5-b]indol-2-one), and pyridoindole cytidine (H-pyrido[3,2,5]pyrrolo[2,3-d]pyrimidin-2-one).

As discussed above, the nucleobase may be a protected nucleobase. For example, the nucleobase may be protected in an orthogonal manner from other protecting groups present, meaning that one set of protecting group(s) may be removed, in any order, using reagents and conditions that do not affect the protecting group(s) in other sets. For example, in some embodiments, an adenine nucleobase may be protected with, e.g., a benzoate-protecting group or a benzyl-protecting group. A guanine, in some embodiments, may be protected with a benzoate or isobutyryl protecting group.

The disclosed method may also include one or more of the following steps: orthogonally protecting a 4′ OH of a nucleoside and an amine nitrogen of a nucleobase; oxidizing a 3′OH in the nucleoside to form a carbonyl moiety; converting the 3′ position to an oxime moiety; deprotecting the 4′ OH in a 3′-oxime modified nucleoside; selectively reducing the 3′-oxime to an amine; and converting a 3′-amine to a protected amine.

The method may further include one or more purifications of intermediates such as after performing one or more steps of the method. In some embodiments, chromatography purification is performed after 4, 3, 2, 1 or none of the method steps. In embodiments, no chromatography purification is necessary.

Nucleosides

### Adenosine Nucleobases

The method of the disclosure may include synthesis of a 2′-F, 3′-amine nucleoside having an adenosine nucleobase (“2′-F, 3′-N A-nucleoside”). In this case, the starting material can be a 2′-F, 3′-OH A-nucleoside, where the amine of the adenosine has been protected with a nitrogen protecting group, e.g., a Bz moiety and the 5′-OH is orthogonally protected with an alcohol protecting group, e.g., TBDMS. The 3′-OH is converted to a ketone, and may be then converted to an oxime without isolation. The orthogonally protected 5′-OH and/or the nitrogen protecting group may optionally be selectively deprotected, and optionally isolated, e.g., via crystallization. The optionally deprotected compound may then be converted to the hydroxylamine, which may be isolated crude and converted to the 2′-F, 3′-N A-nucleoside. Additional optional steps include orthogonally protecting the 3′-amine, e.g., with a MMTr, and optionally orthogonally protecting the amine of the adenosine, e.g., with a Bz moiety. As will be understood alternate protecting groups, as disclosed herein, may be used. In some embodiments, these additional protecting steps are carried out on a crude 2′-F, 3′-N A-nucleoside.

The method of the disclosure may include synthesis of a 2′-MOE, 3′-amine nucleoside having an adenosine nucleobase (“2′-MOE, 3′-N A-nucleoside”). In this case, the starting material can be a 2′-MOE, 3′-OH A-nucleoside, where the amine of the adenosine has been protected with a nitrogen protecting group, e.g., a Bz moiety and the 5′-OH is orthogonally protected with an alcohol protecting group, e.g., TBDMS. The 3′-OH is converted to a ketone, which can be isolated via crystallization, or it may be then converted to an oxime without isolation. In some embodiments, the oxime is isolated, e.g., via crystallization. The orthogonally protected 5′-OH and/or the nitrogen protecting group may optionally be selectively deprotected, and optionally isolated, e.g., via crystallization. The optionally deprotected compound may then be converted to the hydroxylamine, which can be isolated via crystallization, or may be isolated crude and converted to the 2′-MOE, 3′-N A-nucleoside. Additional optional steps include orthogonally protecting the 3′-amine, e.g., with an MMTr, and optionally orthogonally protecting the amine of the adenosine, e.g., with a Bz moiety. As will be understood alternate protecting groups, as disclosed herein, may be used. In some embodiments, these additional protecting steps are carried out on a crude 2′-MOE, 3′-N A-nucleoside.

The method of the disclosure may include synthesis of a 2′-OMe, 3′-amine nucleoside having an adenosine nucleobase (“2′-OMe, 3′-N A-nucleoside”). In this case, the starting material can be a 2′-MOE, 3′-OH A-nucleoside, where the amine of the adenosine has been protected with a nitrogen-protecting group, e.g., a Bz moiety and the 5′-OH is orthogonally protected with an alcohol-protecting group, e.g., TBDMS. The 3′-OH is converted to a ketone, and may be then converted to an oxime without isolation. In some embodiments, the oxime is isolated, e.g., via crystallization. The orthogonally protected 5′-OH and/or the nitrogen protecting group may optionally be selectively deprotected, and optionally isolated, e.g., via crystallization. The optionally deprotected compound may then be converted to the hydroxylamine, which can be isolated via crystallization or chromatography, or isolated crude and converted to the 2′-OMe, 3′-N A-nucleoside. Additional optional steps include orthogonally protecting the 3′-amine, e.g., with an MMTr, and optionally orthogonally protecting the amine of the adenosine, e.g., with a Bz moiety. As will be understood alternate protecting groups, as disclosed herein, may be used. In some embodiments, these additional protecting steps are carried out on a crude 2′-OMe, 3′-N A-nucleoside.

### Guanosine Nucleobases

The method of the disclosure may include synthesis of a 2′-F, 3′-amine nucleoside having a guanosine nucleobase (“2′-F, 3′-N G-nucleoside”). In this case, the starting material can be a 2′-F, 3′-OH G-nucleoside, where the amine of the guanosine has been protected with a nitrogen-protecting group, e.g., a Bz or isobutyryl moiety and the 5′-OH is orthogonally protected with an alcohol-protecting group, e.g., TBDMS. The 3′-OH is converted to a ketone, and may be then converted to an oxime without isolation. The orthogonally protected 5′-OH and/or the nitrogen protecting group may optionally be selectively deprotected, and optionally isolated, e.g., via crystallization. The optionally deprotected compound may then be converted to the hydroxylamine, which can be isolated via crystallization, or may be isolated crude and converted to the 2′-F, 3′-N G-nucleoside. In some embodiments, the optionally deprotected oxime is reduced by treatment with NaBH(OAc)₃. Additional optional steps include orthogonally protecting the 3′-amine, e.g., with an MMTr, and optionally orthogonally protecting the amine of the guanosine, e.g., with an isobutyryl moiety. As will be understood alternate protecting groups, as disclosed herein, may be used. In some embodiments, these additional protecting steps are carried out on a crude 2′-F, 3′-N G-nucleoside.

The method of the disclosure may include synthesis of a 2′-MOE, 3′-amine nucleoside having a guanosine nucleobase (“2′-MOE, 3′-N G-nucleoside”). In this case, the starting material can be a 2′-MOE, 3′-OH G-nucleoside, where the amine of the guanosine has been protected with a nitrogen-protecting group, e.g., a Bz or isobutyryl moiety and the 5′-OH is orthogonally protected with an alcohol-protecting group, e.g., TBDMS. The 3′-OH is converted to a ketone, and may be then converted to an oxime without isolation. In some embodiments, the oxime is isolated, e.g., via crystallization. The orthogonally protected 5′-OH and/or the nitrogen protecting group may optionally be selectively deprotected, and optionally isolated, e.g., via crystallization. The optionally deprotected compound may then be converted to the hydroxylamine, which can be isolated via crystallization, or may be isolated crude and converted to the 2′-MOE, 3′-N G-nucleoside. In some embodiments, the optionally deprotected oxime is reduced by treatment with NaBH(OAc)₃. Additional optional steps include orthogonally protecting the 3′-amine, e.g., with an MMTr, and optionally orthogonally protecting the amine of the guanosine, e.g., with a Bz or isobutyryl moiety. As will be understood alternate protecting groups, as disclosed herein, may be used. In some embodiments, these additional protecting steps are carried out on a crude 2′-MOE, 3′-N G-nucleoside.

The method of the disclosure may include synthesis of a 2′-OMe, 3′-amine nucleoside having a guanosine nucleobase (“2′-OMe, 3′-N G-nucleoside”). In this case, the starting material can be a 2′-MOE, 3′-OH G-nucleoside, where the amine of the guanosine has been protected with a nitrogen-protecting group, e.g., a Bz or isobutyryl moiety and the 5′-OH is orthogonally protected with an alcohol-protecting group, e.g., TBDMS. The 3′-OH is converted to a ketone, and may be then converted to an oxime without isolation. In some embodiments, the oxime is isolated, e.g., via crystallization. The orthogonally protected 5′-OH and/or the nitrogen protecting group may optionally be selectively deprotected, and optionally isolated, e.g., via crystallization. The optionally deprotected compound may then be converted to the hydroxylamine, which can be isolated via crystallization, or may be isolated crude and converted to the 2′-OMe, 3′-N G-nucleoside. In some embodiments, the optionally deprotected oxime is reduced by treatment with NaBH(OAc)₃. Additional optional steps include orthogonally protecting the 3′-amine, e.g., with an MMTr, and optionally orthogonally protecting the amine of the guanosine, e.g., with a Bz or isobutyryl moiety. As will be understood alternate protecting groups, as disclosed herein, may be used. In some embodiments, these additional protecting steps are carried out on a crude 2′-OMe, 3′-N G-nucleoside.

### Uridine Nucleobases

The method of the disclosure may include synthesis of a 2′-F, 3′-amine nucleoside having a uridine nucleobase (“2′-F, 3′-N U-nucleoside”). In this case, the starting material can be a 2′-F, 3′-OH U-nucleoside, where the 5′-OH is protected with an alcohol-protecting group, e.g., TBDMS. The 3′-OH is converted to a ketone, and may be then converted to an oxime without isolation. The protected 5′-OH may optionally be selectively deprotected, and optionally isolated, e.g., via crystallization. The optionally deprotected compound may then be converted to the hydroxylamine, which may be isolated crude and converted to the 2′-F, 3′-N U-nucleoside. Additional optional steps include orthogonally protecting the 3′-amine, e.g., with an MMTr. As will be understood alternate protecting groups, as disclosed herein, may be used. In some embodiments, these additional protecting steps are carried out on a crude 2′-F, 3′-N U-nucleoside.

The method of the disclosure may include synthesis of a 2′-MOE, 3′-amine nucleoside having a uridine nucleobase (“2′-MOE, 3′-N U-nucleoside”). In this case, the starting material can be a 2′-MOE, 3′-OH U-nucleoside, where the 5′-OH is protected with an alcohol-protecting group, e.g., TBDMS. The 3′-OH is converted to a ketone, and may be then converted to an oxime without isolation. In some embodiments, the oxime is isolated, e.g., via crystallization. The protected 5′-OH and may optionally be selectively deprotected, and optionally isolated, e.g., via crystallization. The optionally deprotected compound may then be converted to the hydroxylamine, which may be isolated crude and converted to the 2′-MOE, 3′-N U-nucleoside. Additional optional steps include protecting the 3′-amine, e.g., with an MMTr. As will be understood alternate protecting groups, as disclosed herein, may be used. In some embodiments, these additional protecting steps are carried out on a crude 2′-MOE, 3′-N U-nucleoside.

The method of the disclosure may include synthesis of a T-OMe, 3′-amine nucleoside having a uridine nucleobase (“2′-OMe, 3′-N U-nucleoside”). In this case, the starting material can be a 2′-MOE, 3′-OH U-nucleoside, where the 5′-OH is protected with an alcohol-protecting group, e.g., TBDMS. The 3′-OH is converted to a ketone, and may be then converted to an oxime without isolation. In some embodiments, the oxime is isolated, e.g., via crystallization. The protected 5′-OH may optionally be selectively deprotected, and optionally isolated, e.g., via crystallization. The optionally deprotected compound may then be converted to the hydroxylamine, which may be isolated crude and converted to the 2′-OMe, 3′-N U-nucleoside. Additional optional steps include orthogonally protecting the 3′-amine, e.g., with an MMTr. As will be understood alternate protecting groups, as disclosed herein, may be used. In some embodiments, these additional protecting steps are carried out on a crude 2′-OMe, 3′-N U-nucleoside.

### Cytidine Nucleobases

The method of the disclosure may include synthesis of a 2′-F, 3′-amine nucleoside having a cytidine nucleobase (“2′-F, 3′-N C-nucleoside”). In this case, the starting material can be a 2′-F, 3′-OH C-nucleoside, where the 5′-OH is protected with an alcohol-protecting group, e.g., TBDMS. The 3′-OH is converted to a ketone, and may be then converted to an oxime without isolation. The protected 5′-OH may optionally be selectively deprotected, and optionally isolated, e.g., via crystallization. The optionally deprotected compound may then be converted to the hydroxylamine, which may be isolated crude and converted to the 2′-F, 3′-N C-nucleoside. Additional optional steps include orthogonally protecting the 3′-amine, e.g., with an MMTr. As will be understood alternate protecting groups, as disclosed herein, may be used. In some embodiments, these additional protecting steps are carried out on a crude 2′-F, 3′-N C-nucleoside.

The method of the disclosure may include synthesis of a 2′-MOE, 3′-amine nucleoside having a cytidine nucleobase (“2′-MOE, 3′-N C-nucleoside”). In this case, the starting material can be a 2′-MOE, 3′-OH C-nucleoside, where the 5′-OH is protected with an alcohol-protecting group, e.g., TBDMS. The 3′-OH is converted to a ketone, and may be then converted to an oxime without isolation. In some embodiments, the oxime is isolated, e.g., via crystallization. The protected 5′-OH and may optionally be selectively deprotected, and optionally isolated, e.g., via crystallization. The optionally deprotected compound may then be converted to the hydroxylamine, which may be isolated crude and converted to the 2′-MOE, 3′-N C-nucleoside. Additional optional steps include protecting the 3′-amine, e.g., with an MMTr. As will be understood alternate protecting groups, as disclosed herein, may be used. In some embodiments, these additional protecting steps are carried out on a crude 2′-MOE, 3′-N C-nucleoside.

The method of the disclosure may include synthesis of a T-OMe, 3′-amine nucleoside having a cytidine nucleobase (“2′-OMe, 3′-N C-nucleoside”). In this case, the starting material can be a T-MOE, 3′-OH C-nucleoside, where the 5′-OH is protected with an alcohol-protecting group, e.g., TBDMS. The 3′-OH is converted to a ketone, and may be then converted to an oxime without isolation. In some embodiments, the oxime is isolated, e.g., via crystallization. The protected 5′-OH may optionally be selectively deprotected, and optionally isolated, e.g., via crystallization. The optionally deprotected compound may then be converted to the hydroxylamine, which may be isolated crude and converted to the 2′-OMe, 3′-N C-nucleoside. Additional optional steps include orthogonally protecting the 3′-amine, e.g., with an MMTr. As will be understood alternate protecting groups, as disclosed herein, may be used. In some embodiments, these additional protecting steps are carried out on a crude 2′-OMe, 3′-N C-nucleoside.

The present methods afford a more simple and efficient synthesis of a 3′-N modified nucleoside enabling the production of nucleoside monomers to be carried out on a commercial batch scale, such as, for example, on a scale of 500 g, 1 kg, 2 kg, 3 kg, 4 kg, 5 kg, or more of 3′-N modified nucleoside monomers.

In some embodiments, the present methods provide for improved yield and more facile synthetic conditions compared to other synthetic procedures, such as methods performed through an azide intermediate.

In some embodiments, the synthetic scheme comprises one or more of the following steps:

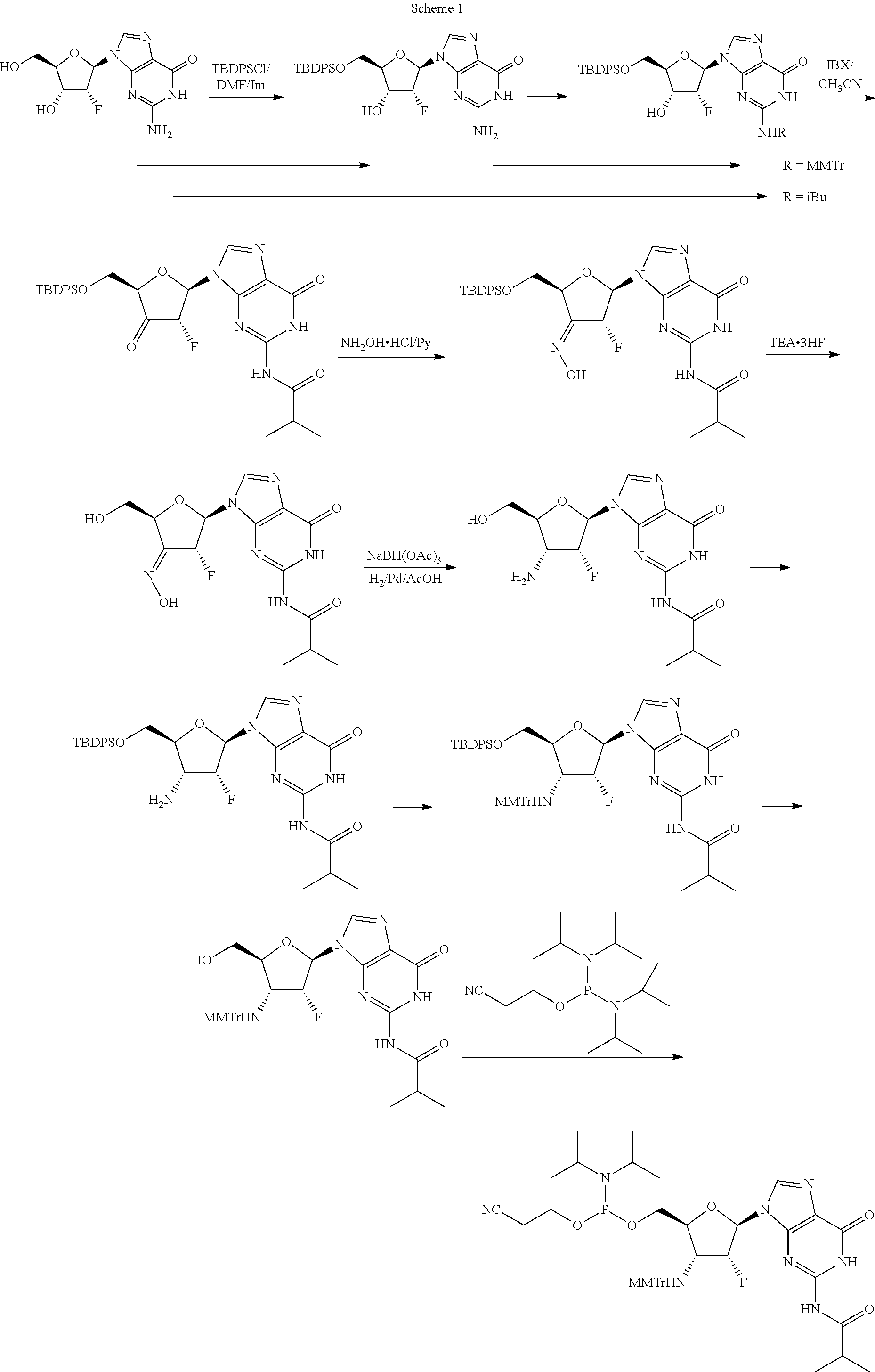

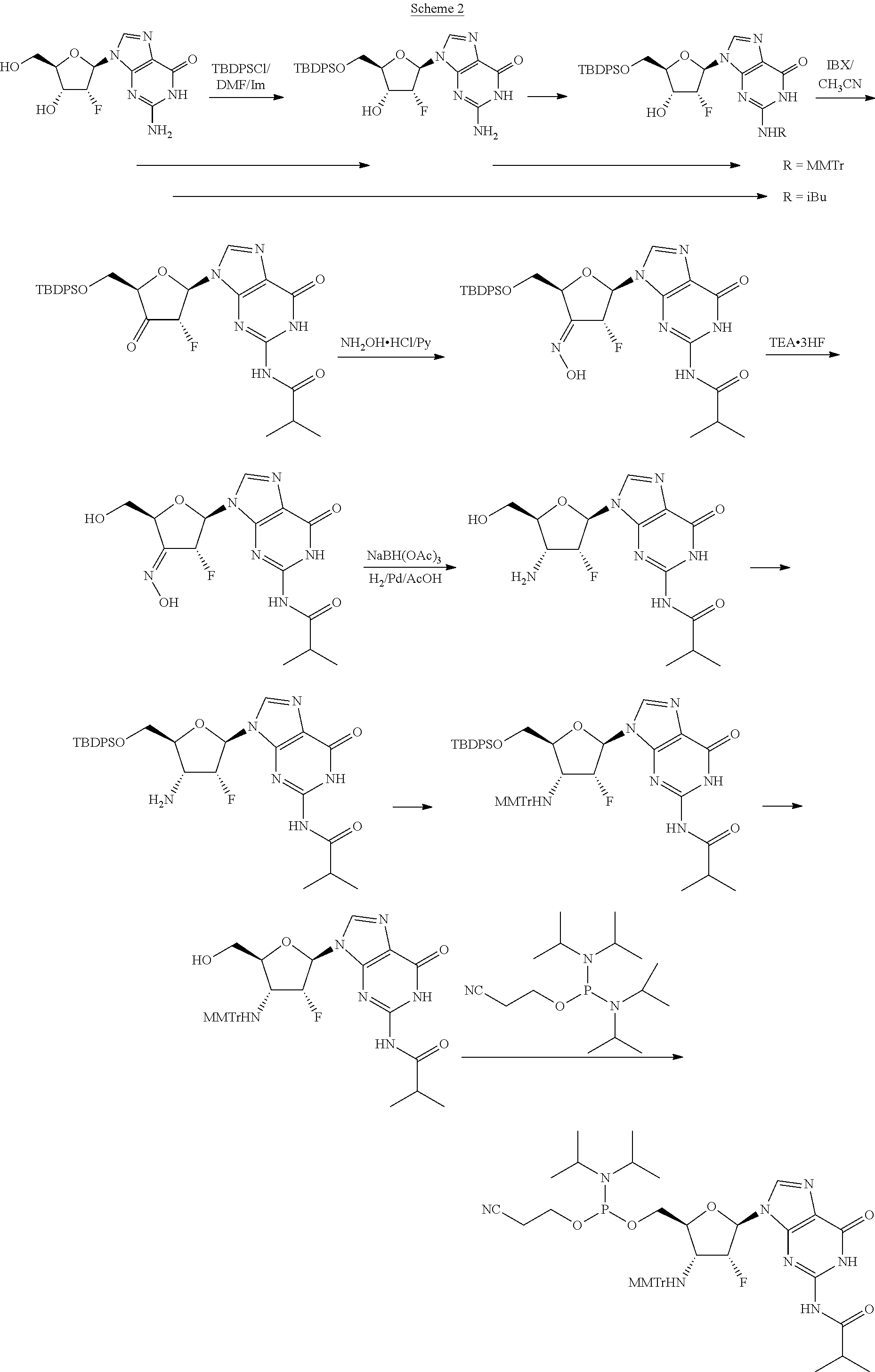

The following Scheme 3 and 4 demonstrates the overall reduction in impurities and isomers, along with the increase in yield of embodiments of the present disclosure compared to a traditional azide synthetic route.

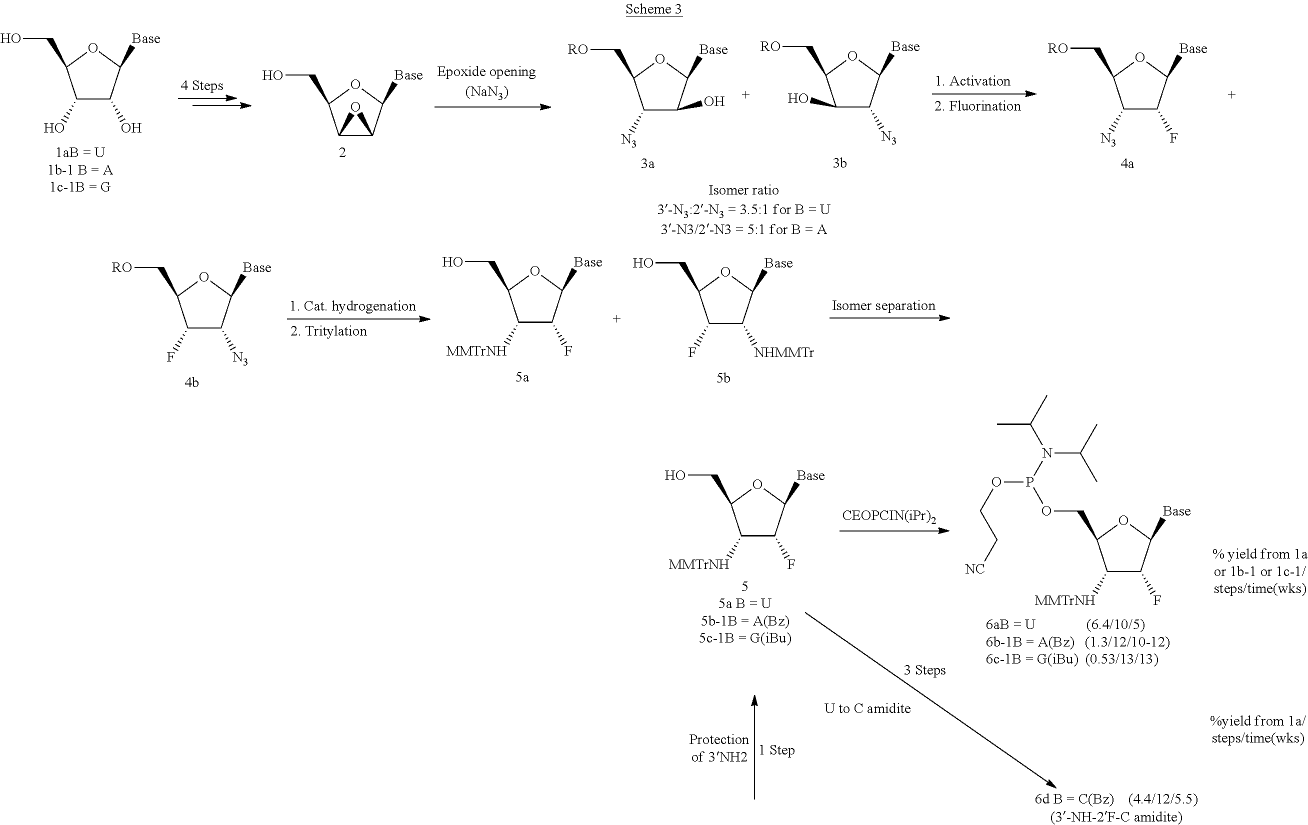

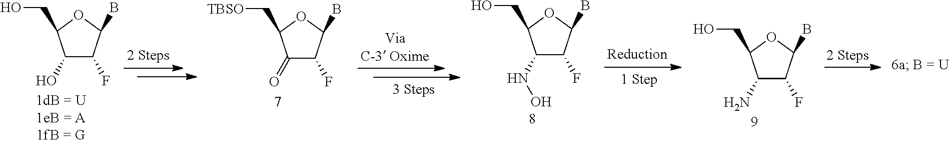

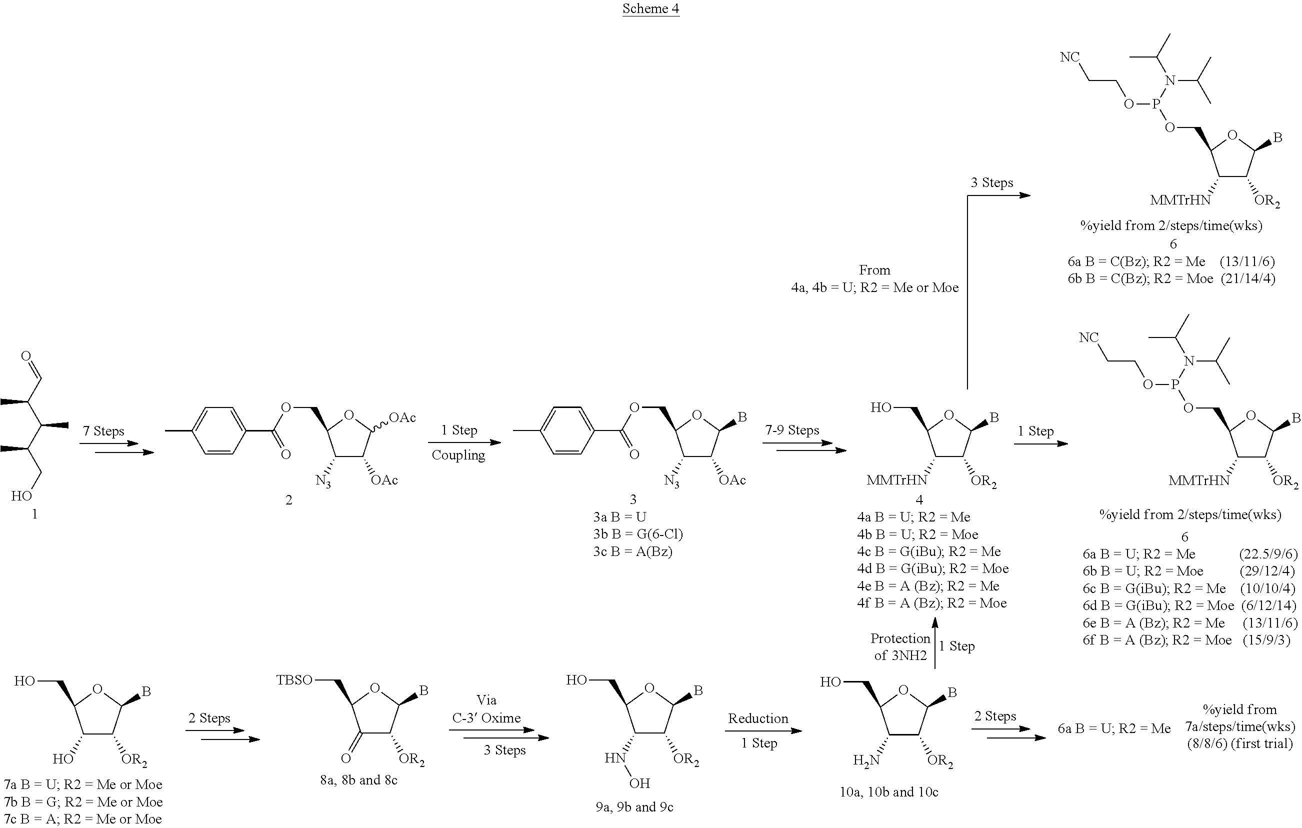

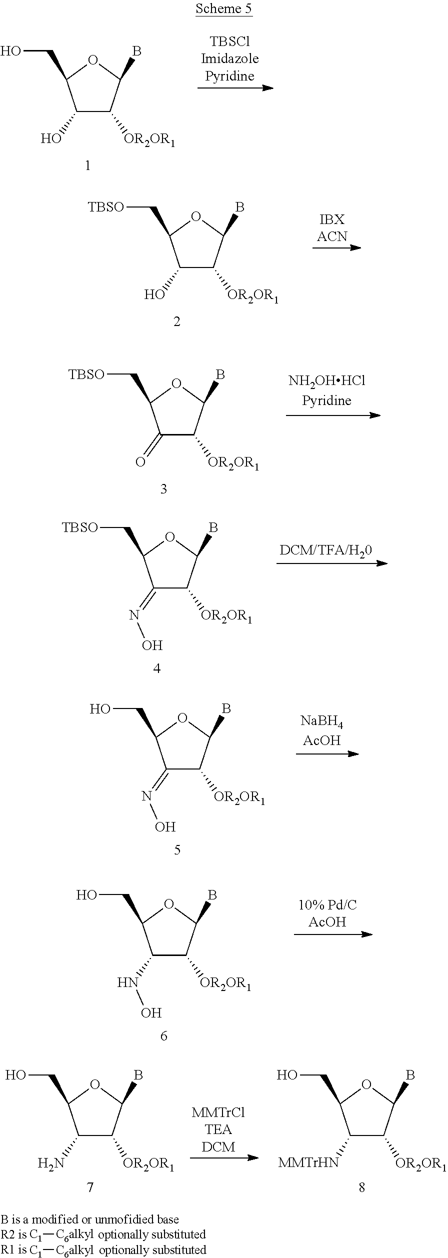

Provided herein also includes novel compounds, such as those represented by any of Formulas (I), (II), and (III).

### Novel Intermediates

For example, in some embodiments, the oxime intermediate can be represented by the following formula (I):

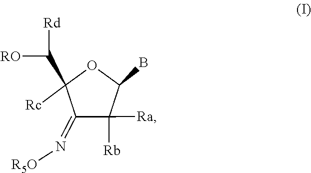

wherein
B is an optionally protected nucleobase; R is H, a counterion or a protecting group, PG; Ra and Rb are each independently selected from the group consisting of H, F, R¹, OR¹, OPG and OR²OR¹; Rc is selected from the group consisting of H, R¹, OPG, OR¹ and N(R₉)₂; Rd is H or R¹; R₅ is H or a C₁₋₆alkyl group (optionally substituted with an aryl group, such as phenyl) and R₉ is independently H or a C₁₋₆alkyl. In some embodiments, at least one of Ra and Rb is not H.

In some embodiments, R is a protecting group, such as a silyl protecting group. In some embodiments, Ra is not OH or OP. In some embodiments, Rb is H. In some embodiments, Rc is H. In some embodiments, Rd is H. In some embodiments, R⁵ is H.

For example, in some embodiments, the reduced oxyamine intermediate can be represented by the following formula (II):

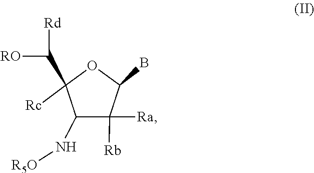

wherein
B is an optionally protected nucleobase; R is H, a counterion or a protecting group, PG; Ra and Rb are each independently selected from the group consisting of H, F, R¹, OR¹, OPG and OR²OR¹; Rc is selected from the group consisting of H, R¹, OPG, OR¹ and N(R₉)₂; Rd is H or R¹; R₅ is H or a C₁₋₆alkyl group (optionally substituted with an aryl group, such as phenyl) and R₉ is independently H or a C₁₋₆alkyl. In some embodiments, the, when R is a protecting group, this group is removed prior to reduction of the oxime moiety. In some embodiments, at least one of Ra and Rb is not H.

In some embodiments, the nucleoside is represented by the following formula (I′) or (II′):

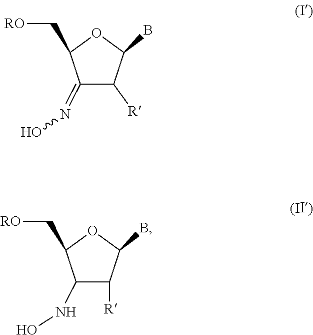

wherein B is a nucleobase, R is H, a counterion, or a protecting group, R′ is F, OR¹ or OR²OR¹, R¹ is a C₁₋₃alkyl or fluoroalkyl, and R² is a C₁₋₅alkylene or fluoroalkylene. The nucleoside and method described herein can be used for the synthesis of the oligonucleotides including ASOs and siRNAs.

For example, in some embodiments, the resulting product can be represented by the following (III):

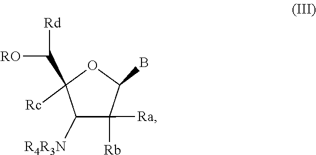

wherein
B is an optionally protected nucleobase; R is H, a counterion or a protecting group, PG; Ra and Rb are each independently selected from the group consisting of H, F, R¹, OR¹, OPG and OR²OR¹; Rc is selected from the group consisting of H, R¹, OPG, OR¹ and N(R₉)₂; Rd is H or R¹, R³ is PG or OPG, and R⁴ is H, OAc, or Ac, or R³ and R⁴ together form a protecting group, such as a cyclic protecting group cPG, wherein R¹ is C₁₋₃alkyl optionally substituted with one or more fluoro or PG, R² is C₁₋₅alkylene optionally substituted with one or more fluoro, each R₉ is independently H or a C₁₋₆alkyl. In some embodiments, at least one of Ra and Rb is not H.

In some embodiments, the variables for formulae (I), (II), or (III) are the same as disclosed above in the section titled “Synthetic Routes.”

### EXAMPLES

### Example 1: Procedure for preparation of 3′-NH-MMTr-2′-O-MOE-A-(Bz)-5′-OH

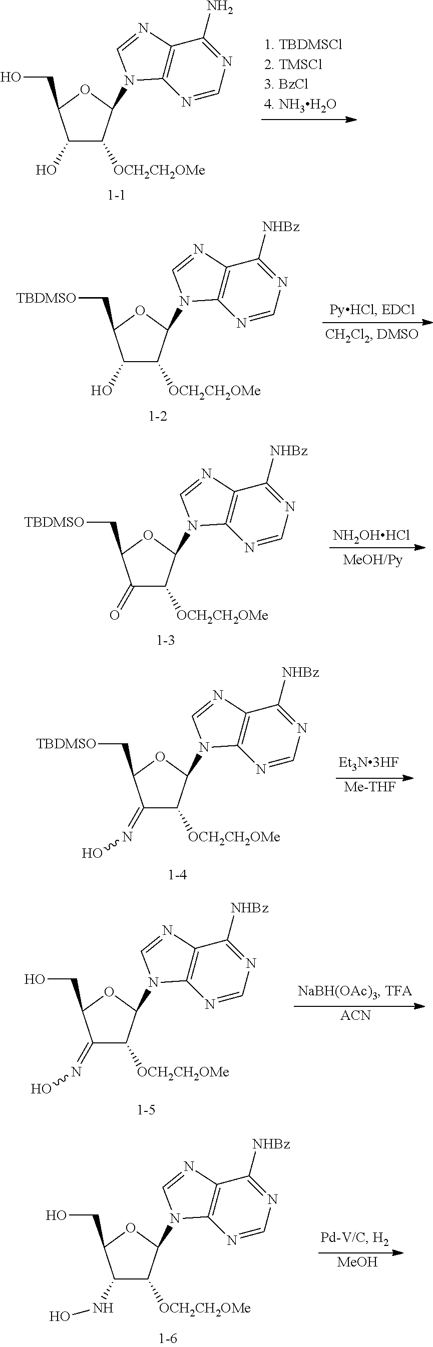

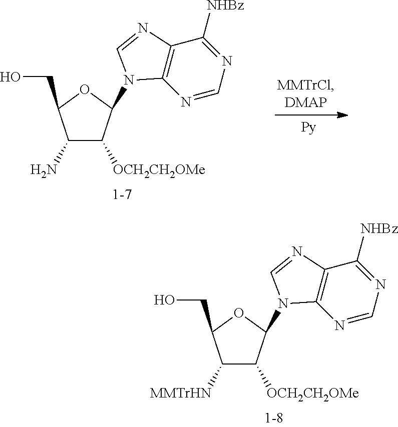

### Synthesis of 1-2

A mixture of 1-1 (30 g, 92.30 mmol) and Pyridine (300 mL) was stirred at 0-5° C. for 15 min. A solution of TBDMSCl (27.80 g, 184.60 mmol) in DCM (30 mL) was added drop wise at 0-5° C., stirred at 25° C. for 16 h. TMSCl (12.02 g, 110.76 mmol) was added at 0° C., stirred at 25° C. for 2 h. BzCl (13.61 g, 96.92 mmol) was added at 0° C., stirred at 25° C. for 2 h. H₂O was added drop wise at 0° C., stirred at 25° C. for 1 h. NH₃·H₂O was added at 25° C. The reaction was extracted with DCM, washed with H₂O, dried with Na₂SO₄, concentrated. Crude 1-2 was obtained as off-white oil (33 g, 65.8% yield). ¹H-NMR (400 MHz, d₆-DMSO) δ 11.16 (s, 1H), 8.70 (s, 1H), 8.58 (s, 1H), 7.99 (d, J=6.4 Hz, 2H), 7.58-7.62 (m, 1H), 7.47-7.52 (m, 2H), 6.11 (d, J=5.2 Hz, 1H), 5.21 (d, J=5.6 Hz, 1H), 4.53-4.56 (m, 1H), 4.31-4.33 (m, 1H), 3.96-3.97 (m, 1H), 3.83 (m, 1H), 3.69-3.75 (m, 2H), 3.60 (m, 1H), 3.36-3.39 (m, 2H), 3.10 (s, 3H), 0.82 (s, 9H), 0.00 (s, 6H), LC-MS ESI m/z: found 544.25 [M+H]⁺.

### Synthesis of 1-3

A solution of Py·HCl (29.61 g, 257.48 mmol) and DMSO (15 mL) in anhydrous DCM (30 mL) was stirred at 20-30° C. for 30 min. Then this mixture was added drop wise into a solution of EDCl (115.13 g, 809.25 mmol), 1-2 (100 g, 183.92 mmol) in anhydrous DCM over 1 h at 20-30° C., the mixture was stirred at 25° C. for 1 h. The reaction solution was telescoped to next step without workup.

### Synthesis of 1-4

MeOH (1000 mL) and Pyridine (110.35 g, 1.47 mol) were added into the solution obtained last step below 20° C., followed by addition of NH₂OH·HCl (31.95 g, 459.8 mmol) in portions below 20° C. The reaction was stirred at 25° C. for 1 h. H₂O was added drop wise below 20° C. From the separate phases, the organic phase was collected, and the aqueous phase was washed with DCM. The combined organic phase was washed with H₂O, 20% AcOH, brine, dried with Na₂SO₄. The organic phase was concentrated under vacuum below 45° C. Crude 1-4 was obtained (193.78 g), which would be telescoped to next step without further purification.

### Synthesis of 1-5

Et₃N·3HF (193 mL) was added drop wise into a solution of crude 1-4 (193.78 g, 347.90 mmol) in 2-MeTHF (1930 mL) below 15° C., then stirred at 30° C. for 3 h. H₂O (1930 mL) was added drop wise below 20° C., and stirred at 25° C. for 10 min. The aqueous phase was washed with 2-MeTHF. The combined organic phase was washed by sat. NaHCO₃ and H₂O, dried with Na₂SO₄, concentrated under vacuum. The crude product was purified via chromatography column. 1-5 (56.46 g, 69.4% yield) was obtained as a yellow solid. ¹H-NMR (400 MHz, d₄-MeOH) 8.68 (s, 1H), 8.66 (s, 1H), 8.02-8.05 (m, 2H), 7.58-7.60 (m, 1H), 7.49-7.53 (m, 2H), 6.14-6.17 (m, 1H), 5.22 (dd, J₁=6.4 Hz, J₂=1.6 Hz, 1H), 5.05-5.07 (m, 1H), 4.06-4.07 (m, 1H), 3.93 (m, 1H), 3.85-3.86 (m, 2H), 3.60-3.70 (m, 1H), 3.35-3.37 (m, 2H), 3.11 (s, 3H), LC-MS ESI m/z: found 443.2 [M+H]⁺.

### Synthesis of 1-6

TFA (10 mL) was added into a solution of 1-5 (0.95 g, 2.14 mmol) in ACN (10 mL) at 10° C., followed by addition of NaBH(OAc)₃ (912 mg, 4.30 mmol) at 5° C. in portions, the reaction was stirred at 10° C. for 2 h. MeOH (5 mL) was added into the reaction, the reaction was stirred at 20° C. for 15 h, concentrated, and purified via chromatography column. 1-6 (0.46 g, 48.2% yield) was obtained as a solid. ¹H-NMR (400 MHz, d₆-DMSO) 8.85 (s, 1H), 8.84 (s, 1H), 8.10-8.12 (m, 2H), 7.70-7.74 (m, 1H), 7.60-7.64 (m, 2H), 6.42 (d, J=5.2 Hz, 1H), 5.05 (m, 1H), 4.55-4.57 (m, 1H), 4.47-4.50 (m, 1H), 3.82-3.86 (m, 2H), 3.69-3.70 (m, 1H), 3.41-3.43 (m, 2H), 3.09 (s, 3H), LC-MS ESI m/z: found 445 [M+H]⁺.

### Synthesis of 1-7

Pt-V/C (0.23 g) was added into a solution of 1-6 (0.46 g, 1.04 mmol) in MeOH (5 mL) at 25° C., the reaction was stirred at 25° C. for 20 h under H₂ (50 PSI). The reaction was filtered and concentrated, crude 1-7 (0.36 g, 81.2% yield) was obtained as a solid. LC-MS ESI m/z: found 429 [M+H]⁺.

### Synthesis of 1-8

MMTrCl (312 mg, 1.01 mmol) and DIPEA (218 mg, 1.69 mmol) were added into a solution of 1-7 (0.36 g, 0.84 mmol) in THF (4 mL), the reaction was stirred at 35° C. for 1 h. The reaction was quenched by H₂O (5 mL) and then 1N HCl was added to adjust pH to 7-8. The aqueous phase was separated and aqueous was extracted by THF. The combined THF solution was then concentrated and the residue was purified via chromatography column. 1-8 was obtained as a solid. ¹H-NMR (400 MHz, CDCl₃) 9.06 (s, 1H), 8.71 (s, 1H), 8.34 (s, 1H), 8.02-8.04 (m, 2H), 7.60 (m, 1H), 7.49-7.53 (m, 6H), 7.40-7.43 (m, 2H), 7.19-7.21 (m, 6H), 6.73-6.75 (d, J=8.8 Hz, 2H), 6.11 (d, J=2 Hz, 1H), 4.07-4.10 (m, 2H), 3.90 (m, 1H), 3.74 (s, 3H), 3.55 (m, 1H), 3.40-3.42 (m, 3H), 3.20-3.24 (m, 2H), 2.98-3.00 (m, 1H), 2.87-2.88 (m, 1H), LC-MS ESI m/z: found 701 [M+H]⁺.

### Example 2

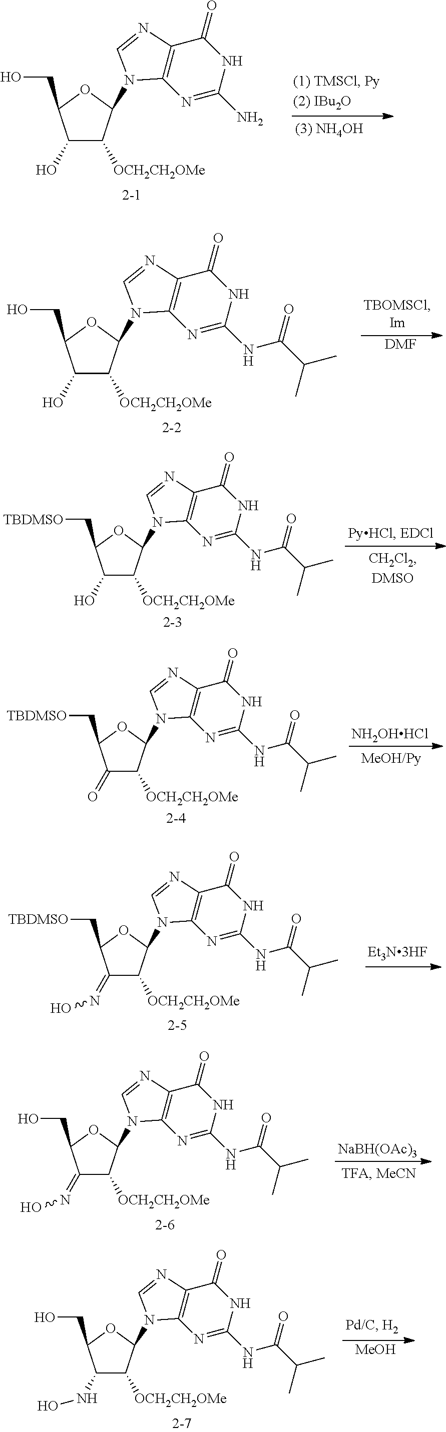

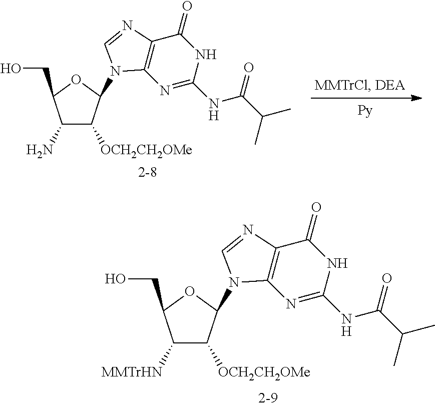

### Synthesis of 2-2

To the 2′-O-Methoxyethyl-guanosine (2-1, 100 g) in flask was added pyridine (400 mL) and CH₂Cl₂ (600 mL). After cooling flask to 0-5° C., TMSCl (3.5 eq.) was added dropwise into reaction solution. Reaction solution was stirred at 25° C. for 2 h. The reaction mixture was cooled to 0-5° C. Isobutyric anhydride (2 eq.) was added into reaction. The mixture was stirred at 25° C. for 0.5 h. The mixture was processed: water was added into flask below 20° C. aq. NH₄OH was added into flask below 20° C. The mixture was stirred at 20-25° C. for 30 min. The mixture was stirred at 25° C. for another 12 h. The aqueous layer was separated and extracted with DCM:MeOH=10:1. The organic layers were separated and combined. The combined organic layers were concentrated under reduced pressure. After addition of toluene, the mixture was concentrated under vacuum. After addition of 1,1,1,3,3,3-Hexafluoro-2-Propanol (HFIP) into flask dropwise at 0° C., MTBE was added into the solution dropwise at 25° C. Crystallization was observed immediately. The mixture was stirred at 25° C. for 1 h. The mixture was filtered and washed filter cake with 300 mL MTBE. The filter cake was collected and dried at 40° C. under vacuum to afford 2-2 as a white solid 106.6 g, 58.5% (yield corrected by assay). ¹H-NMR (400 MHz, d₆-DMSO) δ12.10 (bs, 1H), 11.68 (bs, 1H), 8.29 (s, 1H), 5.90 (d, J=6.4 Hz, 1H), 5.14 (d, J=4.4 Hz, 1H), 5.08 (t, J=5.6 Hz, 1H), 4.42-4.41 (m, 1H), 4.30-4.29 (m, 1H), 4.19-4.11 (m, 1H), 3.94-3.93 (m, 1H), 3.68-3.53 (m, 4H), 3.16 (s, 3H), 2.79-2.75 (m, 1H), 1.12 (d, J=6.8 Hz, 6H). ¹³C-NMR (100 MHz, d₆-DMSO): 180.6, 155.3, 149.3, 148.7, 138.0, 120.4, 86.5, 84.9, 82.0, 71.5, 69.3, 61.6, 58.4, 35.2, 19.2. LC-MS ESI m/z: found 412 [M+H]⁺, 410 [M−H]⁻.

### Synthesis of 2-3

To a solution of 2-2 (50 g, Net amount: 33.11 g, 80.48 mmol) and imidazole (16.44 g, 241.44 mmol, 3.0 eq) in DMF (400 mL) was added a solution of TBDMSCl (13.34 g, 88.53 mmol, 1.1 eq) in DMF (100 mL) dropwise at 20-25° C. The reaction was stirred for 30 min at 20-25° C. The reaction was cooled to 5° C. and quenched by H₂O. The mixture was washed with methylcyclohexane. The aqueous phase was extracted with EA. The combined organic layer was washed by H₂O, dried over Na₂SO₄ and concentrated to give colorless oil. A mixture of EA/Methylcyclohexane was added into the oil and the mixture was stirred at 20-25° C. for 4 h. The solid 2-3 was collected by filtration and dried under reduced pressure at 40° C. to afford white solid (28.0 g, 66.7% yield uncorrected by assay). ¹H-NMR (400 MHz, d₆-DMSO) δ 12.04 (brs, 1H), 11.63 (brs, 1H), 8.11 (s, 1H), 5.85 (d, J=6.0 Hz, 1H), 5.15 (d, J=4.8 Hz, 1H), 4.34-4.33 (m, 1H), 4.23-4.20 (m, 1H), 3.91-3.90 (m, 1H), 3.74-3.69 (m, 3H), 3.64-3.55 (m, 1H), 3.37-3.35 (m, 1H), 3.11 (s, 3H), 2.70 (hept, J=6.8 Hz, 1H), 1.06 (d, J=6.4 Hz, 6H), 0.84 (s, 9H), 0.00 (s, 6H). ¹³C-NMR (100 MHz, d₆-DMSO): 180.6, 155.3, 149.3, 148.7, 137.5, 120.6, 85.7, 85.1, 82.0, 71.7, 69.6, 69.1, 63.4, 58.5, 35.2, 26.3, 19.3, 18.5, 5.0. LC-MS ESI m/z: found 526 [M+H]⁺, 524 [M−H]⁻.

### Synthesis of 2-4

To the Py·HCl (19.23 g, 166.45 mmol) in flask was added DCM (1.25 L). The solution was concentrated to 600 mL. After addition of another DCM (600 mL) into flask, 2-3 (125.0 g, 237.78 mmol) and EDCl (113.96 g, 594.45 mmol) was charged into reaction solution. DMSO was added into reaction solution dropwise. The mixture was stirred for 1 h at 20-25° C. The reaction solution was telescoped to next step directly. LC-MS ESI m/z: found 524 [M+H]⁺, 540 [M+18−H]⁻ for hydrate compound, 542 [M+18+H]⁺ for hydrate compound.

### Synthesis of 2-5

A mixture of MeOH/Py (1250 mL/312 mL) was added into the solution of 2-4 from last step. After charging NH₂OH·HCl (82.69 g, 1.19 mol) into the solution, the reaction mixture was stirred for 1 h at 20-25° C. DCM was added into the mixture and reaction mixture was washed by H₂O. Organic layer was separated, dried over Na₂SO₄ and concentrated to afford a crude oil as a mixture of 2-5 isomer (0.7/0.3, E/Z not determined), (140.0 g, 2-step yield: 109%, uncorrected by assay). ¹H-NMR (400 MHz, d₆-DMSO) δ 12.15 (s, 1H), 11.73 (s, 1H), 8.20 (s, 1H*0.7), 8.05 (s, 1H*0.3), 6.0 (d, J=3.2 Hz, 1H*0.3), 5.90 (d, J=6.8 Hz, 1H*0.7), 5.17-5.15 (m, 1H), 4.97 (s, 1H*0.7), 4.79 (s, 1H*0.3), 4.03-3.75 (m, 5H), 3.39-3.37 (m, 2H), 3.12 (s, 3H), 2.82-2.79 (m, 1H), 1.14-1.09 (m, 6H), 0.85 (s, 9H), 0.04-0.02 (m, 6H). ¹³C-NMR (100 MHz, d₆-DMSO): 183.0, 158.1, 157.6, 151.6, 151.2, 140.0, 122.9, 89.0, 87.7, 82.2, 81.8, 81.3, 79.4, 74.2, 74.1, 72.4, 72.1, 66.6, 64.9, 60.7, 60.6, 43.2, 28.5, 28.4, 21.6, 21.5, 20.9, 20.8, −2.76, −2.80. LC-MS ESI m/z: found 537 [M−H]⁻.

### Synthesis of 2-6

After addition dropwise of Et₃N·3HF (140 mL, 3.3 eq) to a solution of 2-5 (140 g crude form last step) in 2-Me-THF (1.4 L), the reaction was stirred for 18 h at 20-25° C. 2-Me-THF (2.8 L) was added into the reaction and reaction mixture was washed by H₂O. Organic layer was dried by Na₂SO₄ and concentrated to afford crude product. Triturated the crude product with MTBE for 0.5 h at 20-25° C. The solid was collected by filtration and dried under reduced pressure to afford yellow solid 2-6 (110 g crude, yield: 100%, uncorrected by assay). ¹H-NMR (400 MHz, d₆-DMSO) δ11.53 (brs, 2H), 8.42 (s, 1H), 5.91 (d, J=6.8 Hz, 1H), 5.23-5.20 (m, 1H), 4.96 (s, 1H), 3.95-3.68 (m, 5H), 3.42-3.41 (m, 2H), 3.39 (s, 3H), 2.83-2.81 (m, 1H), 1.17 (d, J=4.0 Hz, 6H). ¹³C-NMR (100 MHz, d₆-DMSO): 180.6, 180.4, 172.5, 156.4, 155.2, 150.0, 149.4, 148.8, 138.2, 136.6, 124.3, 120.4, 85.1, 79.8, 79.3, 71.7, 71.6, 70.1, 69.8, 60.4, 58.4, 58.3, 35.1, 21.5, 19.3. LC-MS ESI m/z: found 425 [M+H]⁺, 423 [M−H]⁻.

### Synthesis of 2-7

To NaBH(OAc)₃ (468.92 g, 2.21 mol) in MeCN (6.26 L) was added TFA (939 mL) at 0-5° C. A solution of 2-6 (313 g, 737.51 mmol) in MeCN/TFA (3.13 L/939 mL) was added dropwise into the flask at 0-5° C. The reaction was stirred at 0-5° C. for 1 h. The reaction was quenched by MeOH (1565 mL) and then concentrated to afford crude product. The crude was purified by silica gel column to afford 2-7 as a yellow solid (231.0 g, yield: 74%, uncorrected by assay). ¹H-NMR (400 MHz, d₆-DMSO) δ8.36 (s, 1H), 6.0 (d, J=4.0 Hz, 1H), 4.49 (t, J=12.4 Hz, 1H), 4.12 (d, J=3.2 Hz, 1H), 3.65-3.57 (m, 5H), 3.40-3.38 (m, 2H), 3.17 (s, 3H), 2.77-2.74 (m, 1H), 1.12 (d, J=18.8 Hz, 6H). ¹³C-NMR (100 MHz, d₆-DMSO): 180.6, 172.6, 155.3, 149.3, 148.7, 137.7, 120.4, 86.0, 83.2, 82.0, 71.6, 70.2, 62.7, 62.1, 58.5, 49.0, 35.2, 21.4, 19.2. LC-MS ESI m/z: found 427 [M+H]⁺, 425 [M−H]⁻.

### Synthesis of 2-8

A mixture of 2-7 (50 g crude, from last step) and Pd/C (25 g) in MeOH (1.2 L) and TFA (200 mL) was stirred at 20-25° C. under H₂ (40-45 Psi) atmosphere for 40 h. The reaction was filtered. The filtrate was concentrated to afford crude product oil (79.2 g), which was telescoped to next step directly without purification. ¹H-NMR (400 MHz, d₆-DMSO) δ8.28 (s, 1H), 5.98 (d, J=3.2 Hz, 1H), 5.12 (bs, 1H), 4.27-4.25 (m, 1H), 3.87-3.71 (m, 5H), 3.62-3.53 (m, 1H), 3.47-3.46 (m, 2H), 3.20 (s, 3H), 2.77-2.74 (m, 1H), 1.12 (d, J=18.8 Hz, 6H). ¹³C-NMR (100 MHz, d₆-DMSO): 180.6, 155.3, 148.7, 148.5, 137.9, 120.6, 86.4, 85.3, 83.1, 71.6, 70.1, 60.9, 58.5, 51.6, 46.0, 35.1, 19.3, 9.2. LC-MS ESI m/z: found 411 [M+H]⁺.

### Synthesis of 2-9

To a solution of 2-8 (366 g crude, from last step) in Py/DIEA (5.49 L/1.1 L) was added MMTrCl (303.23 g, 981.95 mmol). The reaction mixture was stirred at 20-25° C. for 0.5 h. The reaction mixture was concentrated to afford crude oil. The crude was dissolved in DCM and washed with H₂O. The organic layer was dried over Na₂SO₄ and concentrated to afford a crude product. The crude was recrystallized from DCM/MTBE at 20-25° C. for three times to afford product 2-9 (221 g, 2-step yield: 55%, uncorrected by assay) as yellow solid. ¹H-NMR (400 MHz, d₆-DMSO) δ12.13 (bs, 1H), 11.39 (bs, 1H), 8.09 (s, 1H), 7.48-7.44 (m, 4H), 7.29-7.21 (m, 6H), 7.20-7.12 (m, 2H), 6.75 (d, J=8.8 Hz, 2H), 5.80-5.76 (m, 1H), 5.09-5.07 (m, 1H), 3.92-3.90 (m, 3H), 3.65 (s, 3H), 3.59-3.51 (m, 1H), 3.23-3.22 (m, 2H), 3.20-3.14 (m, 1H), 3.15 (s, 3H), 3.10-3.01 (m, 1H), 2.84-2.78 (m, 2H), 1.97-1.95 (m, 1H), 1.17-1.13 (m, 6H). ¹³C-NMR (100 MHz, d₆-DMSO): 180.7, 158.1, 155.3, 148.3, 148.0, 147.2, 146.7, 138.1, 137.1, 130.3, 128.6, 128.3, 126.7, 126.6, 120.7, 113.5, 85.6, 84.3, 81.2, 71.5, 70.1, 69.3, 59.8, 58.7, 55.4, 53.4, 35.1, 19.4. LC-MS ESI m/z: found 683 [M+H]⁺, 681 [M−H]⁻.

### Example 3

### Preparation of 2′-Moe-U Nucleoside

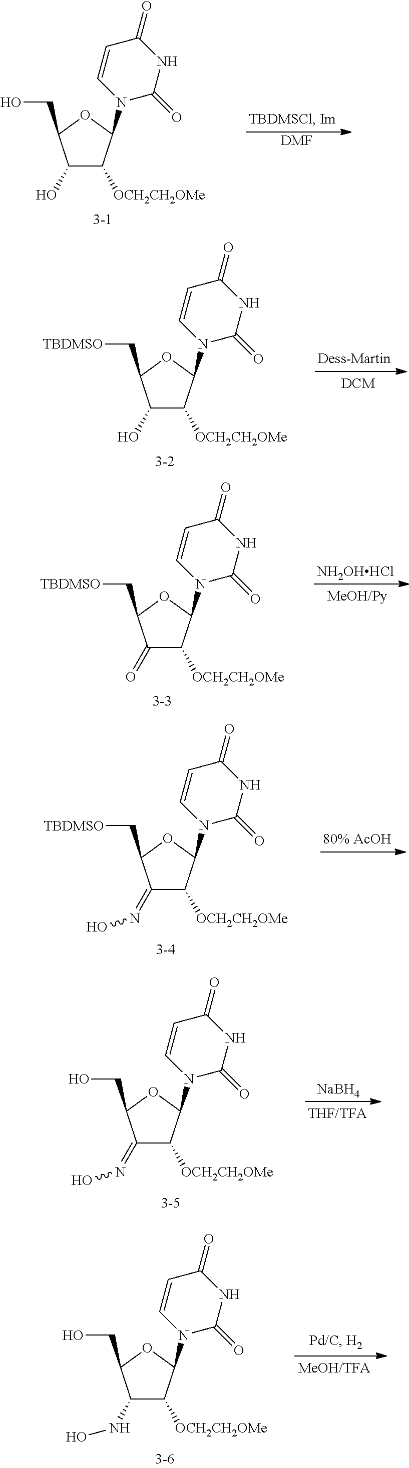

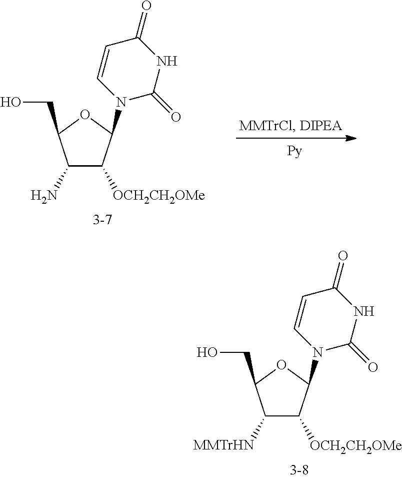

### Synthesis of 3-2

To a solution of 1-[(2R,3R,4R,5R)-3-MOE-4-hydroxy-5-(hydroxymethyl)oxolan-2-yl]-1,2,3,4-tetrahydropyrimidine-2,4-dione (3-1, 708 g, 2.34 mol, 1.0 eq.) in N,N-dimethylformamide (3540 ml) with an inert atmosphere of nitrogen was added imidazole (4.68 mol, 2.0 eq.). Then tert-butyl (chloro)dimethylsilane (3.04 mol, 1.3 eq.) in N,N-dimethylformamide (3540 ml) was followed in several batches at 0 degree and control inner temperature below 15 degree. The resulting solution was stirred for 16 h at room temperature. The reaction was then quenched by methanol. The resulting mixture was concentrated under reduced pressure. The residue was dissolved in dichloromethane. The resulting mixture was washed with H₂O. The mixture was dried over anhydrous sodium sulfate. The solids were filtered out. The resulting mixture was concentrated under reduced pressure. The residue was purified by silica gel column. After concentration, yellowish oil 3-2 was obtained with 99% HPLC Purity. (920 g, Yield: 95%). ¹H-NMR (400 MHz, d₆-DMSO) δ11.30 (s, 1H), 7.73 (d, J=8.2 Hz, 114), 5.75 (d, J=4.4 Hz, 1H), 5.48 (d, J=8.2 Hz, 1H), 5.02 (d, J=6.0 Hz, 1H), 3.99-3.97 (m, 1H), 3.85-3.81 (m, 2H), 3.79-3.75 (m, 1H), 3.67-3.63 (m, 1H), 3.61-3.54 (m, 2H), 3.37 (t, J=9.6 Hz, 2H), 3.14 (s, 3H), 0.81 (s, 9H), 0 (s, 6H).

### Synthesis of 3-3

To a solution of 3-2 (165 g, 0.40 mol, 1.0 eq.) in dichloromethane (1650 ml) with an inert atmosphere of nitrogen was added Dess-Martin periodinane (0.52 eq., 1.3 eq.) in several batches at 0 degree in 10-20 min. The resulting solution was stirred at 20-30 degree until A′ was consumed. The resulting solution was diluted with dichloromethane and H₂O. The resulting mixture was filtered under vacuum and washed with H₂O. The mixture was dried over anhydrous sodium sulfate. The solids were filtered out. The resulting mixture was concentrated under reduced pressure to afford 3-3 as white solid. (167 g, crude Yield: 101%).

### Synthesis of 3-4

To a solution of 3-3 (167 g, 0.4 mol, 1.0 eq.) in methanol/pyridine (2004 ml/501 ml) with an inert atmosphere of nitrogen was added Hydroxylamine hydrochloride (2.0 mol, 5.0 eq.). The resulting solution was stirred at 20-30 degree until 3-3 was consumed. The resulting mixture was concentrated under reduced pressure. The residue was dissolved in dichloromethane. The resulting mixture was washed with water, 15˜20% HOAc and H₂O respectively. The mixture was dried over anhydrous sodium sulfate. The solids were filtered out. The resulting mixture was concentrated under reduced pressure to afford 3-4 as yellowish oil with 92% HPLC purity. (136 g, Yield: 78.5%). MS m/z [M+H]⁺ (ESI): 430.

### Synthesis of 3-5

To a solution of 80% HOAc (795 ml) was added 3-4 (53 g, 0.12 mol, 1.0 eq.). Then the solution was warm to 40 degree and stir at the temperature until IPC showed the C′ was consumed. Concentrate the mixture under vacuum below 40 degree. And charge toluene to swap out HOAc. After workup, 3-5 brown oil was obtained with 85% HPLC purity (54 g, Crude yield>100%).

### Synthesis of 3-6

Into round-bottom flask purged and maintained with an inert atmosphere of nitrogen, was placed 3-5 (2 g, 6.34 mmol, 1.0 eq.), then THF/TFA (20 ml/4 ml) was added. The resulting solution was stirred at 20-30 degree until the reaction was finished. Quench the reaction with methanol (6 ml). The resulting mixture was concentrated under reduced pressure to afford 3-6 as a light yellow oil with 80.5% HPLC purity (7.1 g, Crude yield>100%). ¹H-NMR (400 MHz, d₆-DMSO) δ11.34 (s, 1H), 8.02 (d, J=8.1 Hz, 1H), 7.53 (s, 1H), 5.89 (d, J=4.6 Hz, 1H), 5.66 (d, J=8.1 Hz, 2H), 5.21 (t, J=4.8 Hz, 1H), 4.08 (t, J=5.2 Hz, 1H), 3.96-3.94 (m, 1H), 3.75-3.65 (m, 3H), 3.57-3.49 (m, 2H), 3.46 (t, J=4.6 Hz, 2H), 3.24 (s, 3H). ¹³C-NMR (100 MHz, d₆-DMSO): 163.6, 151.0, 140.8, 102.2, 87.5, 82.5, 81.3, 71.6, 70.0, 61.9, 61.8, 58.6. LC-MS ESI m/z: found 318 [M+H]⁺, 340 [M+Na]⁺.

### Synthesis of 3-7

To a solution of 3-6 (0.5 g, 1.58 mmol, 1.0 eq.) in methanol/trifluoroacetic acid (20:1; 5 ml) was added 10% Palladium on activated carbon (0.5×). The flask was evacuated and flushed 3 times with hydrogen. The resulting solution was stirred at 20-30 degrees until 3-6 was consumed. The solids were filtered out. The resulting mixture was concentrated under reduced pressure to afford crude 3-7 as brown oil with 89.4% HPLC purity (0.7 g, crude yield>100%). MS m/z [M+H]⁺ (ESI): 302.

### Synthesis of 3-8

To a solution of 3-7 (0.7 g, 2.32 mmol, 1.0 eq.) in Pyridine (7 ml) was added DIPEA (10.9 mmol, 4.7 eq.), MMTrCl (3.02 mmol, 1.3 eq.). The mixture was stirred at 20-30 degrees until the 3-7 was consumed. This was followed by concentration and extraction. The crude product was purified by column. Concentrate the fraction to afford 3-8 as off white solid with 96.7% HPLC purity (0.7 g, yield: 52.6%). ¹H-NMR (400 MHz, d₆-DMSO) δ11.27 (d, J=1.8 Hz, 1H), 8.00 (d, J=8.2 Hz, 1H), 7.47-7.44 (m, 4H), 7.33 (d, J=8.8 Hz, 2H), 7.28 (t, J=7.6 Hz, 4H), 7.21-7.17 (m, 2H), 6.84 (d, J=8.9 Hz, 2H), 5.51-5.48 (m, 2H), 5.18 (t, J=3.8 Hz, 1H), 4.06-4.01 (m, 2H), 3.87 (d, J=10.0 Hz, 1H), 3.71 (s, 3H), 3.12-3.06 (m, 1H), 2.98 (s, 3H), 2.61 (d, J=10.8 Hz, 1H), 1.31 (d J=4.4 Hz, 1H). LC-MS ESI m/z: found 572 [M−H]⁻.

### Example 4

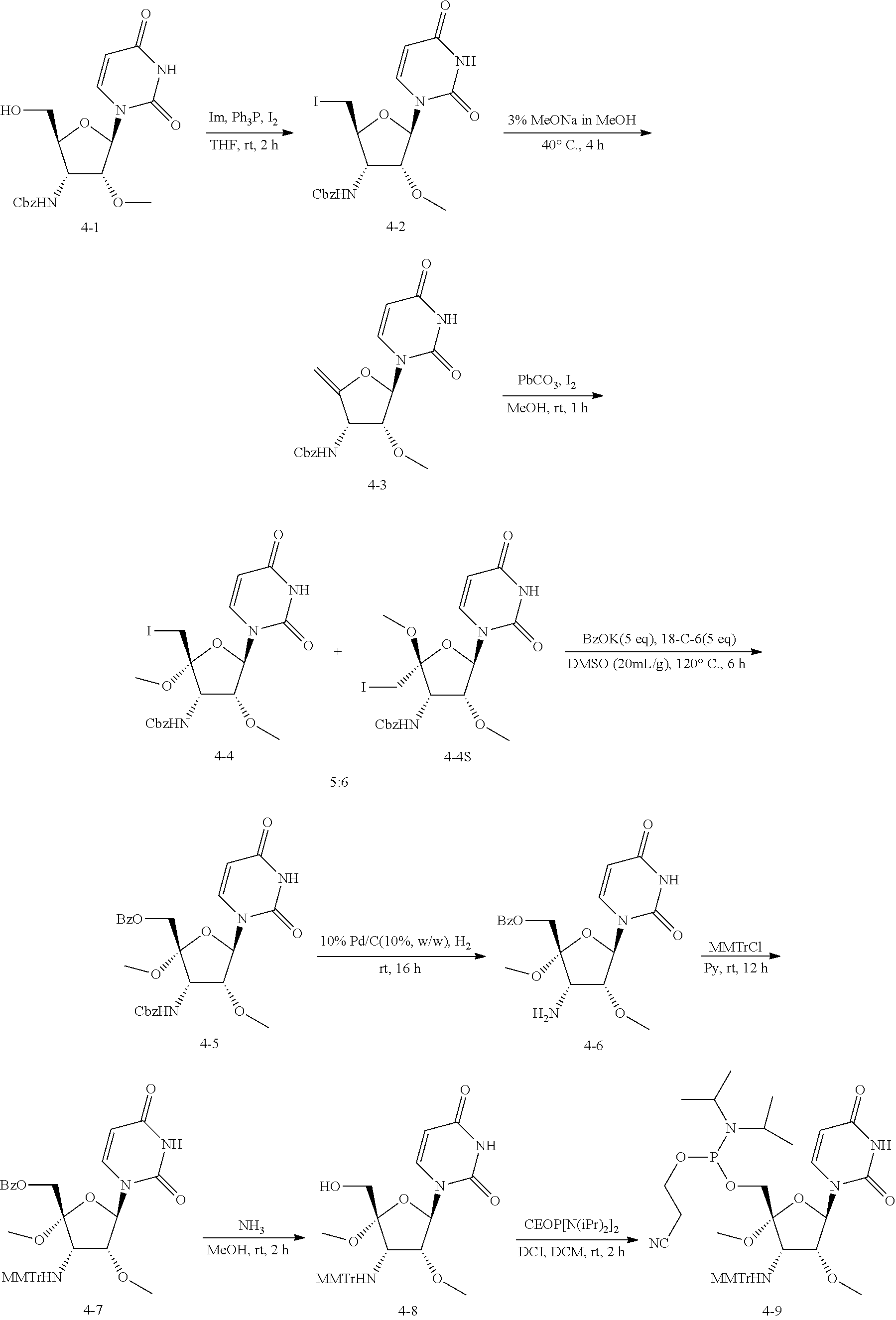

### Synthesis of 4-2

To a solution of 4-1 (40.0 g, 102.2 mmol, 1.00 eq.) in tetrahydrofuran (300 mL) with an inert atmosphere of nitrogen was added imidazole (27.8 g, 408.3 mmol, 4.00 eq.) and triphenylphosphane (107.2 g, 409.2 mmol, 4.00 eq.) in order at room temperature. Then a solution of iodane (52 g, 204.7 mmol, 2.00 eq.) in tetrahydrofuran (100 mL) was added dropwise with stirring at 0° C. The resulting solution was stirred for 2 h at room temperature, diluted with dichloromethane. The resulting solution was washed with 10% aqueous sodium thiosulfate and water respectively. The organic phase was dried over anhydrous sodium sulfate, filtered, and concentrated under reduced pressure. The residue was applied onto a silica gel column. 42 g (82%) of 4-2 were obtained as a white solid. MS m/z [M+H]+ (ESI): 502.

### Synthesis of 4-3

4-2 (50 g, 99.7 mmol, 1.00 eq.) was dissolved in 500 mL of 3% sodium methanolate in methanol at room temperature. The resulting solution was stirred for 4 h at 40° C. The pH value of the solution was adjusted to 7 with acetic acid. The resulting solution was concentrated under reduced pressure. The residue was purified by Flash-Prep-HPLC. 23 g (62%) of 4-3 were obtained as a white solid. MS m/z [M+H]+ (ESI): 396. Synthesis of 4-4

### Synthesis of 4-4

To a solution of 4-3 (20 g, 53.6 mmol, 1.00 eq.) in methanol (200 mL) was added lead carbonate (28.6 g, 107.1 mmol, 2.00 eq.) and iodane (27.2 g, 107.1 mmol, 2.00 eq.) in order at room temperature. The resulting solution was stirred for 1 h at room temperature. The resulting solution was concentrated under reduced pressure. The residue was diluted with 500 mL of dichloromethane and washed with 10% aqueous sodium thiosulfate. The organic phase was dried over anhydrous sodium sulfate, filtered, and concentrated under reduced pressure. The residue was purified by Flash-Prep-HPLC. 20 g (crude) of 4-4/4-4S (5:6) were obtained as a yellow solid. MS m/z [M+H]+ (ESI): 532.

### Synthesis of 4-5

To a solution of 4-4/4-4S (20 g, 37.6 mmol, 1.00 eq.) in dimethyl sulfoxide (400 mL) with an inert atmosphere of nitrogen was followed by the addition of potassium benzoate (30.1 g, 188.1 mmol, 5.00 eq.) and 18-C-6 (49.7 g, 188.3 mmol, 5.00 eq.) in order at room temperature. The resulting solution was stirred for 6 h at 120° C. Then the reaction was cooled to 25° C. and quenched water. The resulting solution was extracted with ethyl acetate. The organic phases were combined, washed with saturated aqueous sodium chloride. The organic phase was dried over anhydrous sodium sulfate, filtered, and concentrated under reduced pressure. The residue was by Flash-Prep-HPLC. 3.32 g (37%) of 4-5 were obtained as a brown solid and 3.29 g (40%) of 4-5S were obtained as a light yellow solid.

### Synthesis of 4-6

To a solution of 4-5 (5.0 g, 9.5 mmol, 1.00 eq.) in tetrahydrofuran/triethylamine (50 mL, 4:1) was added 10% palladium on activated carbon (500 mg) at room temperature. The flask was evacuated and flushed five times with hydrogen. The resulting solution was stirred for 16 h at room temperature. The solid was filtered out. The filtrate was concentrated under reduced pressure. 3.02 g (81%) of 4-6 were obtained as a light yellow solid. MS m/z [M+H]+ (ESI): 392.

### Synthesis of 4-7

To a solution of 4-6 (4.0 g, 10.2 mmol, 1.00 eq.) in 40 mL of pyridine with an inert atmosphere of nitrogen was added 1-(chlorodiphenylmethyl)-4-methoxybenzene (3.3 g, 10.7 mmol, 1.05 eq.) at room temperature. The resulting solution was stirred for 12 h at room temperature. The reaction was quenched by the addition of 2 mL of methanol. The resulting mixture was concentrated under reduced pressure. The resulting solution was diluted with 200 mL of dichloromethane. The resulting solution was washed with water. The organic phase was dried over anhydrous sodium sulfate, filtered, and concentrated under reduced pressure. The residue was applied onto a silica gel column. 4.72 g (70%) of 4-7 were obtained as a light yellow solid. MS m/z [M−H]⁻ (ESI): 662.

### Synthesis of 4-8

4-7 (5.0 g, 7.5 mmol, 1.00 eq.) was dissolved in 50 mL of the addition of ammonia/methanol (7 M) at room temperature. The resulting solution was stirred for 2 h at room temperature. The resulting mixture was concentrated under reduced pressure. The residue was applied onto a silica gel column. 3.32 g (79%) of 4-8 were obtained as a yellow solid. MS m/z [M−H]⁻ (ESI): 558. 1H NMR (DMSO-d₆, 300 Hz): δ1.44 (m, 1H), 2.67-2.71 (m, 1H), 2.77-2.80 (s, 3H), 3.39-3.42 (s, 3H), 3.43-3.45 (m, 1H), 3.70-3.79 (s, 3H), 3.81-3.95 (m, 2H), 5.16-5.019 (t, J=4.2 Hz, 1H), 5.56-5.59 (d, J=8.1 Hz, 1H), 5.67 (s, 1H), 6.83-6.86 (m, 2H), 7.17-7.46 (m, 8H), 7.49-7.47 (m, 4H), 7.57-7.59 (d, J=8.1 Hz, 1H), 11.32 (s, 1H).

### Synthesis of 4-9

To a solution of 4-8 (2.2 g, 3.93 mmol, 1.00 eq.) in dichloromethane (33 mL) with an inert atmosphere of nitrogen was followed by the addition of bis(diisopropylamino)(2-cyanoethoxy)phosphine (1.3 g, 4.31 mmol, 1.10 eq.) and 4, 5-dicyanoimidazole (464 mg, 3.93 mmol, and 1.00 eq.) at room temperature. The resulting solution was stirred for 2 h at room temperature. The resulting solution was diluted with 200 mL of dichloromethane. The resulting solution was washed with aqueous sodium bicarbonate and saturated aqueous sodium chloride respectively. The organic phase was dried over anhydrous sodium sulfate, filtered, and concentrated under reduced pressure. The residue was purified by Flash-Prep-HPLC. The fractions were diluted with dichloromethane and dried over anhydrous sodium sulfate. The solid was filtered out. The filtrate was concentrated until no residual solvent left at 30° C. under reduced pressure. 1.82 g (61%) of 4-9 was obtained as a light yellow solid. MS m/z [M−H]⁻ (ESI): 758. 1H NMR (DMSO-d₆, 300 Hz): δ 0.98-1.13 (m, 12H), 1.22-1.65 m, 1H), 2.69-2.78 (m, 4H), 2.82-2.83 (s, 2H), 3.31-3.36 (s, 3H), 3.49-3.54 (m, 4H), 3.70 (s, 4H), 3.85-4.05 (m, 2H), 5.49-5.56 (m, 1H), 5.61-5.70 (m, 1H), 6.82-6.87 (m, 2H), 7.18-7.51 (m, 13H), 11.40 (s, 1H). ³¹PNMR (DMSO-d₆, 300 Hz): δ 147.14, 147.95.

### Example 5

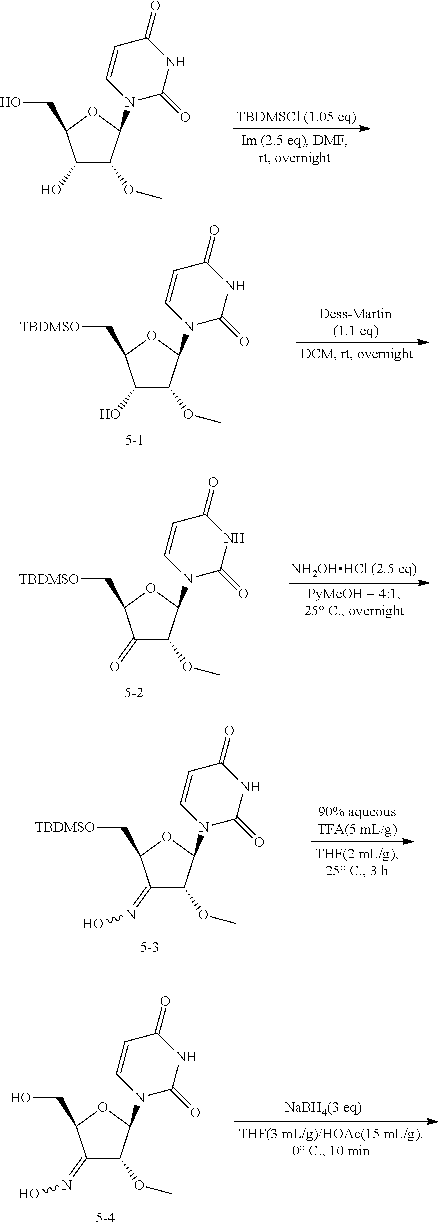 [SMILES: CO[C@@H]1[C@H](O)[C@@H](CO)O[C@H]1n1ccc(=O)[nH]c1=O ; CO[C@@H]1[C@H](O)[C@@H](CO[Si](C)(C)C(C)(C)C)O[C@H]1n1ccc(=O)[nH]c1=O ; CO[C@@H]1C(=O)[C@@H](CO[Si](C)(C)C(C)(C)C)O[C@H]1n1ccc(=O)[nH]c1=O ; CO[C@@H]1C(=NO)[C@@H](CO[Si](C)(C)C(C)(C)C)O[C@H]1n1ccc(=O)[nH]c1=O ; CO[C@@H]1C(=NO)[C@@H](CO)O[C@H]1n1ccc(=O)[nH]c1=O]

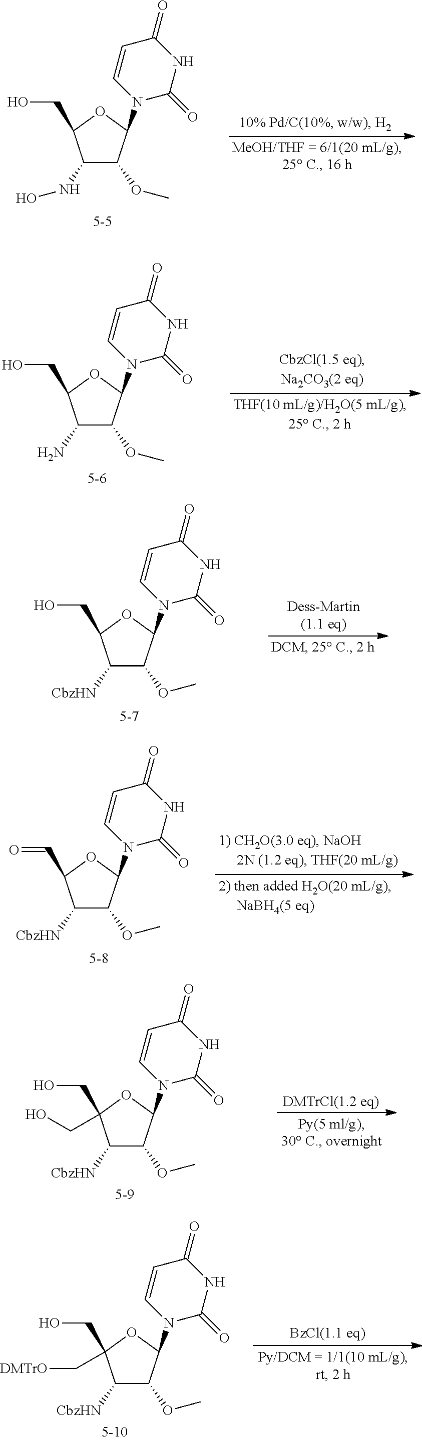

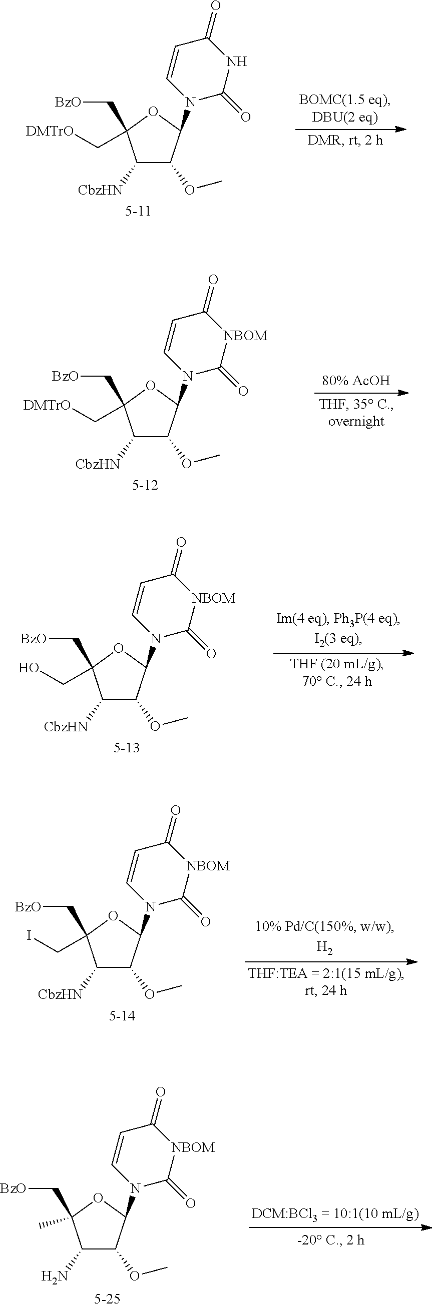

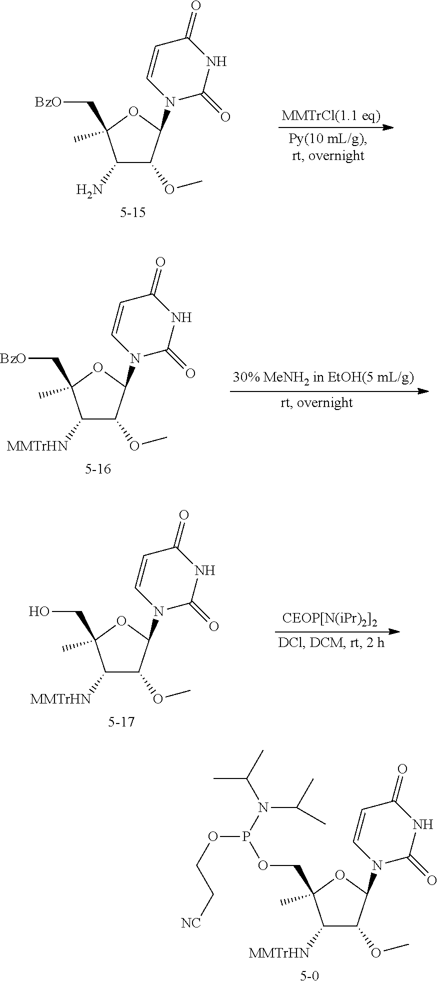

### Synthesis of 5-1

To a solution of 1-[(2R,3R,4R,5R)-4-hydroxy-5-(hydroxymethyl)-3-methoxyoxolan-2-yl]-1,2,3,4-tetrahydropyrimidine-2,4-dione (350 g, 1.36 mol, 1.00 eq.) in 7000 mL of N,N-dimethylformamide was added imidazole (231 g, 3.39 mol, 2.50 eq.) at room temperature. To this was added tert-Butyldimethylsilyl chloride (214 g, 1.42 mol, 1.05 eq.) in several batches and then stirred overnight at room temperature. The reaction was quenched by water and extracted with dichloromethane. The organic phases were combined and washed with saturated aqueous sodium bicarbonate and saturated aqueous sodium chloride respectively. The organic phase was dried over anhydrous sodium sulfate, filtered, and concentrated under reduced pressure. The residue was applied onto a silica gel column. 420 g (83%) of 5-1 were obtained as a white solid. MS m/z [M+H]+ (ESI): 373.

### Synthesis of 5-2

To a solution of 5-1 (840 g, 2.26 mol, 1.00 eq.) in 8400 mL of dichloromethane was added Dess-Martin (1000 g, 2.36 mol, 1.1 eq.) at room temperature. The resulting solution was stirred overnight at room temperature. The resulting solution was diluted with dichloromethane. The resulting solution was washed with saturated aqueous potassium hydrogen carbonate and saturated aqueous sodium chloride respectively. The organic phase was dried over anhydrous sodium sulfate, filtered, and concentrated under reduced pressure. 1200 g (crude) of 5-2 were obtained as a white solid. MS m/z [M+H]+ (ESI): 371. This crude product was used in next step directly without further purification.

### Synthesis of 5-3

To a solution of 5-2 (840 g, 2.27 mol, 1.00 eq.) in the mixture of pyridine and methanol (12500 mL, 4:1) was added hydroxylamine hydrochloride (400 g, 2.50 eq.). The resulting solution was stirred overnight at 25° C., and then concentrated under reduced pressure. The residue was dissolved in 30000 mL of dichloromethane and washed with water and saturated aqueous sodium chloride respectively. The organic phase was dried over anhydrous sodium sulfate, filtered, and concentrated under reduced pressure. The residue was applied onto a silica gel column. 612 g (70%) of 5-3 were obtained as a white solid. MS m/z [M+H]+ (ESI): 386.

### Synthesis of 5-4

5-3 (300 g, 778.2 mmol, 1.00 eq.) was dissolved in 90% aqueous trifluoroacetic acid (3000 mL) at room temperature. The resulting solution was stirred for 3 h at 25° C. The resulting solution was concentrated under reduced pressure. The residue was dissolved in water. The resulting solution was washed with dichloromethane. The aqueous phase was concentrated under reduced pressure, and then purified by Flash-Prep-HPLC. 135 g (64%) of 5-4 were obtained as a brown solid. MS m/z [M+H]+ (ESI): 272.

### Synthesis of 5-5

To a solution of sodium borohydride (21 g, 555.11 mmol, 3.00 eq.) in the mixture of acetic acid/tetrahydrofuran (420 mL, 5:1) was added a solution of 5-4 (50 g, 184.35 mmol, 1.00 eq.) in acetic acid/tetrahydrofuran (420 mL, 5:1) dropwise at 0° C. The resulting solution was stirred for 10 min at 0° C. The reaction was quenched by the addition of 40 mL of water/methanol (1:1). The resulting mixture was concentrated under reduced pressure. The residue was purified by Flash-Prep-HPLC. 12.7 g (60%) of 5-5 were obtained as a white solid. MS m/z [M+H]+ (ESI): 274.

### Synthesis of 5-6

To a solution of 5-5 (60 g, 219.59 mmol, 1.00 eq.) in methanol/trifluoroacetic acid (780 mL, 6:1) was added 10% palladium carbon (6 g) at room temperature. The flask was evacuated and flushed five times with hydrogen. The resulting solution was stirred for 16 h at 25° C. The solid was filtered out. The filtrate was concentrated under reduced pressure. 60 g (crude) of 5-6 were obtained as a yellow solid. MS m/z [M+H]+ (ESI): 258.

### Synthesis of 5-7

To a solution of 5-6 (60 g, 233.24 mmol, 1.00 eq.) in tetrahydrofuran (600 mL) was added a solution of sodium carbonate (49.6 g, 467.97 mmol, 2.00 eq.) in water (300 mL) and benzyl carbonchloridate (60 g, 351.72 mmol, 1.50 eq.) in order. The resulting solution was stirred for 2 h at 25° C. The resulting solution was extracted with 2×600 mL of dichloromethane. The organic phases were combined, dried over anhydrous sodium sulfate, filtered, and concentrated under reduced pressure. The residue was applied onto a silica gel column. 50 g (55%) of 5-7 were obtained as a white solid. MS m/z [M+H]+ (ESI): 392.

### Synthesis of 5-8

To a solution of 5-7 (40 g, 102.20 mmol, 1.00 eq.) in dichloromethane (400 mL) was added Dess-Martin (47.71 g, 112.52 mmol, 1.10 eq.) was added at room temperature. The resulting solution was stirred for 2 h at 25° C. and diluted with 1000 mL of dichloromethane. The resulting solution was washed with 2×500 mL of saturated aqueous sodium thiosulfate, saturated aqueous potassium hydrogen carbonate and saturated aqueous sodium chloride respectively. The organic phase was dried over anhydrous sodium sulfate, filtered, and concentrated under reduced pressure. 40 g (crude) of 5-8 were obtained as a white solid. This crude was used in next step directly without further purification.

### Synthesis of 5-9

To a solution of 5-8 (30 g, 77.05 mmol, 1.00 eq.) in tetrahydrofuran (600 mL), was added 30% saturated aqueous formaldehyde (18.8 g, 626.67 mmol, 3.00 eq.) and 2N sodium hydroxide (46 mL, 1.20 eq.) in order at 0° C. The resulting solution was stirred overnight at 25° C. To this was added water (600 mL) and sodium borohydride (17.58 g, 464.71 mmol, 5.00 eq.) at 0° C. The resulting solution was allowed to react with stirring for an additional 3 h at 25° C. Acetic acid was employed to adjust the pH to 7. The resulting solution was concentrated under reduced pressure. The residue was dissolved in 2-methyltetrahydrofuran and dried over anhydrous sodium sulfate, filtered and concentrated under reduced pressure. The residue was applied onto a silica gel column. 15 g (46%) of 5-9 were obtained as a white solid. MS m/z [M+H]+ (ESI): 422.

### Synthesis of 5-10

To a solution of 5-9 (15 g, 35.61 mmol, 1.00 eq.) in pyridine (75 mL) was added 1-[chloro (4-methoxyphenyl) benzyl]-4-methoxybenzene (13.23 g, 39.06 mmol, 1.20 eq.). The resulting solution was stirred overnight at 30° C. and quenched by the addition of methanol (20 mL). The resulting solution was concentrated under reduced pressure. The residue was dissolved in 500 mL of dichloromethane and washed with saturated aqueous sodium bicarbonate and saturated aqueous sodium chloride respectively. The organic phase was dried over anhydrous sodium sulfate, filtered, and concentrated under reduced pressure. The residue was applied onto a silica gel column. 9 g (35%) of 5-10 were obtained as a white solid. MS m/z [M+H]+ (ESI): 724.

### Synthesis of 5-11

To a solution of 5-10 (13 g, 17.95 mmol, 1.00 eq.) in the mixture of pyridine/dichloromethane (250 mL, 1:1) was added benzoyl chloride (3.78 g, 26.85 mmol, 1.50 eq.) dropwise with stirring at 0° C. in 10 min. The resulting solution was stirred for 2 h at room temperature. Then the reaction was quenched by the addition of methanol (10 mL) and concentrated under reduced pressure. The residue was dissolved with 1000 mL of dichloromethane and washed with water and saturated aqueous sodium chloride respectively. The organic phase was dried over anhydrous sodium sulfate, filtered, and concentrated under reduced pressure. The residue was purified by Flash-Prep-HPLC. 13 g (87%) of 5-11 were obtained as a yellow solid. MS m/z [M+H]+ (ESI): 828.

### Synthesis of 5-12

To a solution of 5-11 (14 g, 16.9 mmol, 1.00 eq.) in N, N-dimethylformamide (150 mL) was added 1, 8-diazabicyclo [5.4.0] undec-7-ene (5.18 g, 20.55 mmol, 2.00 eq.) and [(chloromethoxy) methyl] benzene (3.17 g, 20.25 mmol, 1.20 eq.) in order. The resulting solution was stirred for 2 h at room temperature, and then concentrated under reduced pressure. The residue was dissolved in dichloromethane and washed with water. The organic phase was dried over anhydrous sodium sulfate, filtered, and concentrated under reduced pressure. 15 g (crude) of 5-12 were obtained as a yellow solid. MS m/z [M+H]+ (ESI): 948. This crude was used in next step directly without further purification.

### Synthesis of 5-13

To a solution of 5-12 (15 g, 15.8 mmol, 1.00 eq.) in tetrahydrofuran (30 mL) was added 80% acetic acid (150 mL, in water). The resulting solution was stirred overnight at 35° C., and then concentrated under reduced pressure. The residue was purified by Flash-Prep-HPLC. 7.5 g (73%) of 5-13 were obtained as a white solid. MS m/z [M+H]+ (ESI): 646.

### Synthesis of 5-14

To a solution of 5-13 (9.2 g, 14.25 mmol, 1.00 eq.) in tetrahydrofuran (90 mL) was added 1H-imidazole (3.822 g, 57.04 mmol, 4.00 eq.) and triphenylphosphane (14.95 g, 57.00 mmol, 4.00 eq.) in order. This was followed by the addition of a solution of iodine (7.2644 g, 28.62 mmol, 2.00 eq.) in tetrahydrofuran (90 mL) dropwise with stirring at room temperature. The resulting solution was stirred for 24 h at 70° C. The reaction was cooled to 25° C. and diluted with 500 mL of ethyl acetate. The resulting solution was washed with saturated aqueous sodium thiosulfate and saturated aqueous sodium chloride respectively. The organic phase was dried over anhydrous sodium sulfate, filtered, and concentrated under reduced pressure. The residue was applied onto a silica gel column. 5 g (46%) of 5-14 were obtained as a yellow solid. MS m/z [M+H]+ (ESI): 756.

### Synthesis of 5-25

To a solution of 5-14 (6 g, 1.99 mmol, 1.00 eq.) in the mixture of tetrahydrofuran/triethylamine (90 mL, 2:1) was added 10% palladium carbon (9 g). The flask was evacuated and flushed five times with hydrogen. The resulting solution was stirred for 24 h at room temperature. The solid was filtered out. The filtrate was concentrated under reduced pressure. The residue was applied onto a silica gel column. 4.3 g (crude) of 5-25 were obtained as yellow oil. MS m/z [M+H]+ (ESI): 496.

### Synthesis of 5-15

To a solution of 5-25 (3 g, 6.06 mmol, 1.00 eq.) in dichloromethane (30 mL) was added trichloroborane (3 mL, 2.00 eq.) dropwise with stirring at −20° C. in 5 min. The resulting solution was stirred for 2 hours at −20° C. and quenched by the addition of ammonia, and then concentrated under reduced pressure. The residue was purified by Flash-Prep-HPLC. 2.25 g (99%) of 5-15 were obtained as a white solid. MS m/z [M+H]+ (ESI): 376.

### Synthesis of 5-16

To a solution of 5-15 (800 mg, 2.13 mmol, 1.00 eq.) in the mixture of pyridine (8 mL) and triethylamine (1 ml) was added 1-(chlorodiphenylmethyl)-4-methoxybenzene (720 mg, 2.33 mmol, 1.10 eq.) at room temperature. The resulting solution was stirred overnight at room temperature. The reaction was quenched by the addition of methanol (2 mL) and concentrated under reduced pressure. The residue was dissolved in 150 mL of dichloromethane and washed with saturated aqueous sodium bicarbonate. The organic phase was dried over anhydrous sodium sulfate, filtered, and concentrated under reduced pressure. The residue was purified by Flash-Prep-HPLC. 1 g (72%) of 5-16 was obtained as a white solid. MS m/z [M+H]+ (ESI): 648.

### Synthesis of 5-17

5-16 (1.6 g, 2.48 mmol, 1.00 eq.) was dissolved in 8 mL of 30% methylamine ethanol and stirred overnight at room temperature. The resulting mixture was concentrated under reduced pressure. The residue was purified by Flash-Prep-HPLC. 1.1 g (85%) of 5-17 was obtained as a white solid. MS m/z [M+H]+ (ESI): 544. 1HNMR (DMSO, 400 MHz) 11.24 (s, 1H), 7.85 (d, J=8.0 Hz, 1H), 7.49 (m, 4H), 7.39 (m, 2H), 7.30 (m, 4H), 7.19 (m, 2H), 6.85 (d, J=3.2 Hz, 2H), 5.57 (s, 1H), 5.52 (d, J=8.0 Hz, 1H), 5.01 (t, J=4.0 Hz, 1H), 3.77 (m, 1H), 3.70 (s, 3H), 3.63 (m, 1H), 3.30 (m, 1H), 2.91 (s, 3H), 2.66 (d, J=8.8 Hz, 1H), 1.29 (s, 3H), 1.27 (m, 1H).

### Synthesis of 5-0

To a solution of 5-17 (1.12 g, 2.07 mmol, 1.00 eq.) in dichloromethane (12 mL) was added 3-(bis(diisopropylamino)phosphinooxy)propanenitrile (688 mg, 2.28 mmol, 1.10 eq.) and 1H-imidazole-4,5-dicarbonitrile (243 mg, 2.07 mol, 1.00 eq.) in order. The resulting solution was stirred for 2 h at room temperature. The resulting solution was diluted with 100 mL of dichloromethane. The resulting solution was washed with saturated aqueous sodium bicarbonate and saturated aqueous sodium chloride respectively. The organic phase was dried over anhydrous sodium sulfate, filtered, and concentrated under reduced pressure. The residue was purified by Flash-Prep-HPLC. The fractions were diluted with 400 mL of dichloromethane and dried over anhydrous sodium sulfate. The solid was filtered out. The filtrate was concentrated until no residual solvent left at 30° C. under reduced pressure, obtained 1.12 g (73%) of 5-0 as a white solid, which was dried for 5 hours under reduced pressure at 25° C. MS m/z [M−H]− (ESI): 742, 1HNMR (CD₃CN, 400 MHz) 8.89 (bs, 1H), 7.83 (m, 1H), 7.60 (m, 4H), 7.50 (m, 2H), 7.31 (m, 4H), 7.22 (m, 2H), 6.86 (m, 2H), 5.68 (d, J=19.6 Hz, 1H), 5.53 (m, 1H), 4.03 (m, 1H), 3.87 (m, 1H), 3.76 (s, 3H), 3.70 (m, 1H), 3.65˜3.37 (m, 4H), 3.38 (m, 1H), 3.00 (m, 3H), 2.88 (m, 1H), 2.58 (m, 1H), 2.49 (m, 1H), 1.42 (s, 3H), 1.23˜1.05 (m, 12H), P-NMR (DMSO, 162 MHz) 147.46, 146.80.

### Example 6

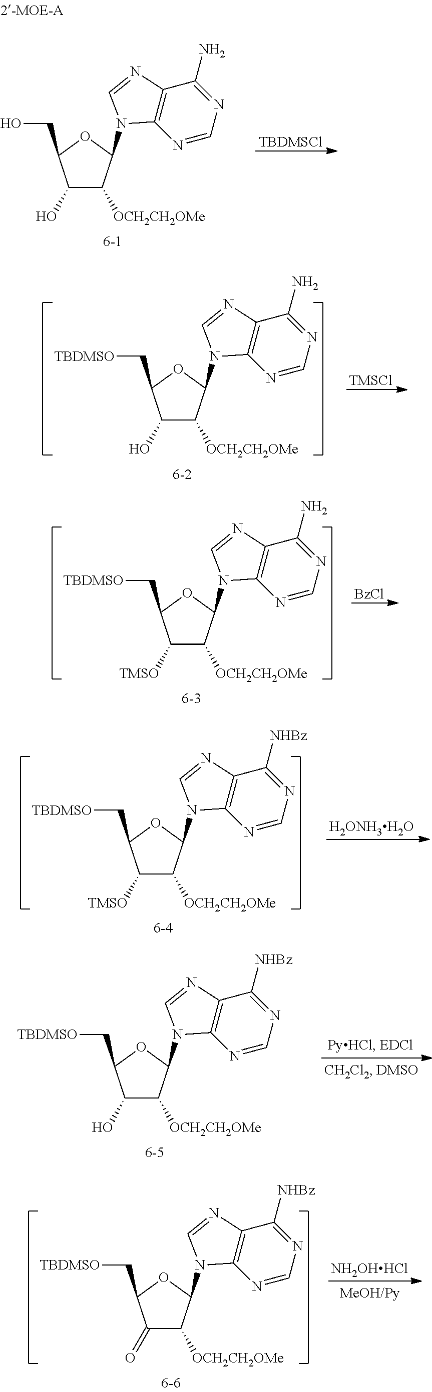

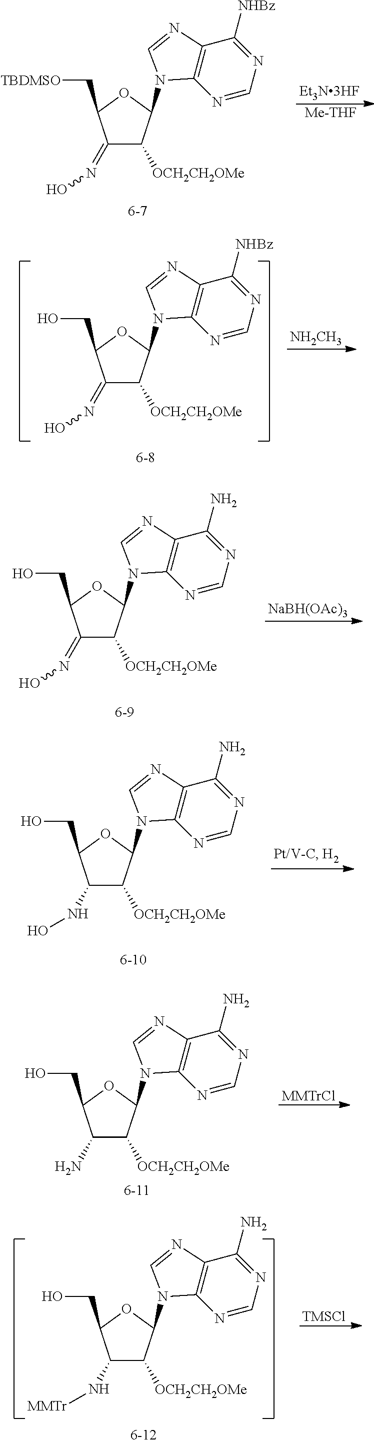

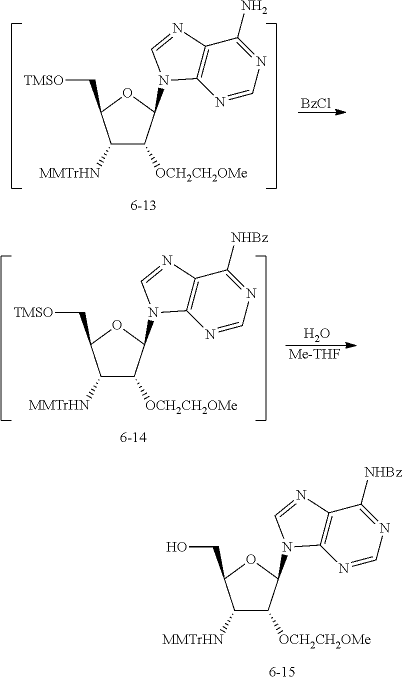

### Preparation of 6-10: ((2S,3R,4R,5R)-5-(6-amino-9H-purin-9-yl)-3-(hydroxyamino)-4-(2-methoxyethoxy)tetrahydrofuran-2-yl)methano

TFA (560 mL) was added into a solution of 6-9 (280 g, 0.83 mol) in THF (2.8 L) at 10° C., followed by addition of NaBH(OAc)₃ (526 g, 2.48 mmol) at 10° C. in portions, the reaction mixture was stirred at 10° C. for 1 h. MeOH (840 mL) was added into the reaction, followed by stirring at 10° C. for 17 h, then neutralized to pH=8 with Et₃N, solid precipitated out. Filtered, 480 g crude 6-10 was obtained. The crude 6-10 was slurred with ACN/H₂O, filtered and dried, 6-10 (259 g, 92.1% yield) was obtained as an off-white solid. ¹H-NMR (400 MHz, d₆-DMSO) δ 8.43 (s, 1H), 8.15 (s, 1H), 7.29-7.56 (m, 1H), 7.31 (S, 2H), 6.039-6.054- (m, 1H), 5.532-5.61 (m, 1H), 4.61-4.64 (m, 1H), 4.13-4.15 (m, 1H), 3.71-3.72 (m, 2H), 3.69-3.70 (m, 2H), 3.61-3.63 (m, 1H), 3.38-3.40 (m, 2H), 3.15 (s, 3H). ¹³C-NMR (100 MHz, d₆-DMSO) δ 58.5, 62.3, 62.9, 70.2, 71.6, 81.3, 83.5, 87.7, 119.8, 140.2, 149.3, 152.9, 156.6. LC-MS ESI m/z: found 341 [M+H]⁺.

### Preparation of 6-11

Pt-V/C (6 g) was added into a solution of 6-10 (30 g, 87.9 mmol) in THE/H₂O (300 mL/150 mL) at 25° C., followed by stirring at 25° C. for 40 h under H₂ (15 Psi). The reaction mixture was filtered and concentrated, crude 6-11 (28 g, 97.9% yield) was obtained as a solid. ¹H-NMR (400 MHz, d₆-DMSO) δ 8.43 (s, 1H), 8.15 (s, 1H), 7.31 (S, 2H), 6.03-6.06 (m, 1H), 5.16-4.18 (m, 1H), 3.71-3.78 (m, 4H), 3.60-3.61 (m, 2H), 3.47-3.50 (m, 2H), 3.24 (s, 3H), 1.63 (s, 1H), LC-MS ESI m/z: found 325 [M+H]⁺.

Preparation of 6-10 was carried out on a batch scale. For the batch, 5.45 Kg 6-10 (purity: 96.9%, assay: 60.6%) was obtained from 6.7 Kg (assay: 82.7%) in 59% yield. The details were summarized in tables below.

| | |
|---|---|
| 1. | Charge E (5.5 kg,) |
| 2. | Charge THF (48 kg, 8.73×) |
| 3. | Adjust batch temperature to 0-10° C. under nitrogen protection. |
| 4. | Drop wise TFA solution (17 kg, 3.09×) via head tank at 0-10° C. |
| 5. | Charge NaBH(AcO)₃ (10.7 kg, 1.95×) in portions at 0-10° C. |
| 6. | Adjust batch temperature to 8-12° C. under nitrogen protection. |
| 7. | Stir at 8-12° C. for 2-3 h under nitrogen protection. |
| 8. | Adjust batch temperature to 0-10° C. under nitrogen protection. |
| 9. | Drop wise MeOH (13.5 kg, 2.45×) via head tank at 0-12° C. |
| | under nitrogen protection. |
| 10. | Adjust batch temperature to 8-12° C. under nitrogen protection. |
| 11. | Stir at 8-12° C. for 16-20 h under nitrogen protection. |
| 12. | Adjust to 0-10° C. under nitrogen protection. |
| 13. | Drop wise TEA (20 kg, 3.64×) via head tank at 0-10° C. under |
| | nitrogen protection, adjust pH to 7-8. |
| 14. | Adjust to 8-12° C. under nitrogen protection. |
| 15. | Stir at 8-12° C. for 2-5 h under nitrogen protection. |
| 16. | Filter material |
| 17. | Filter to dryness |

### Preparation of Compound 6-11

Preparation of 6-11 was carried out on one batch by methods similar to disclosed herein.

### Example 7

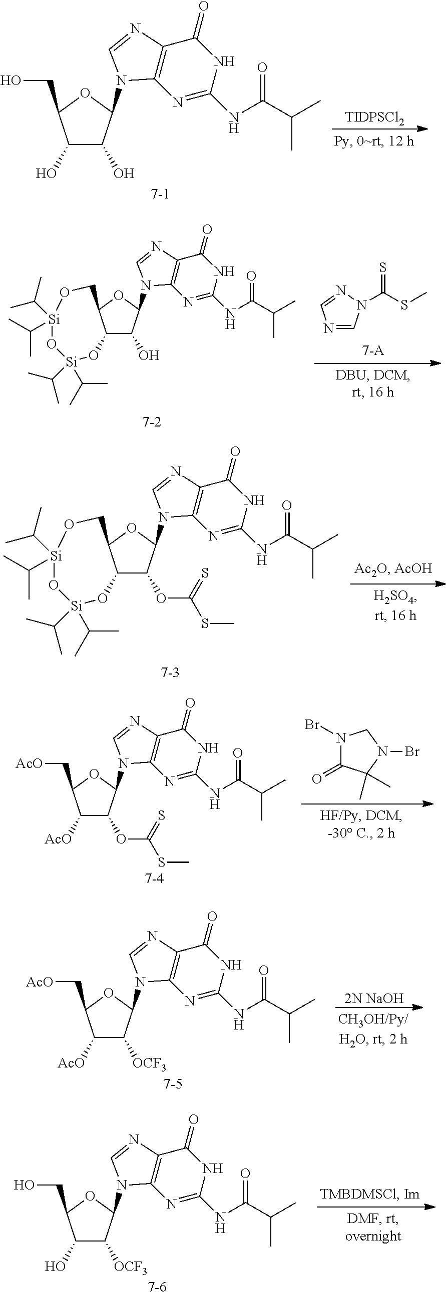

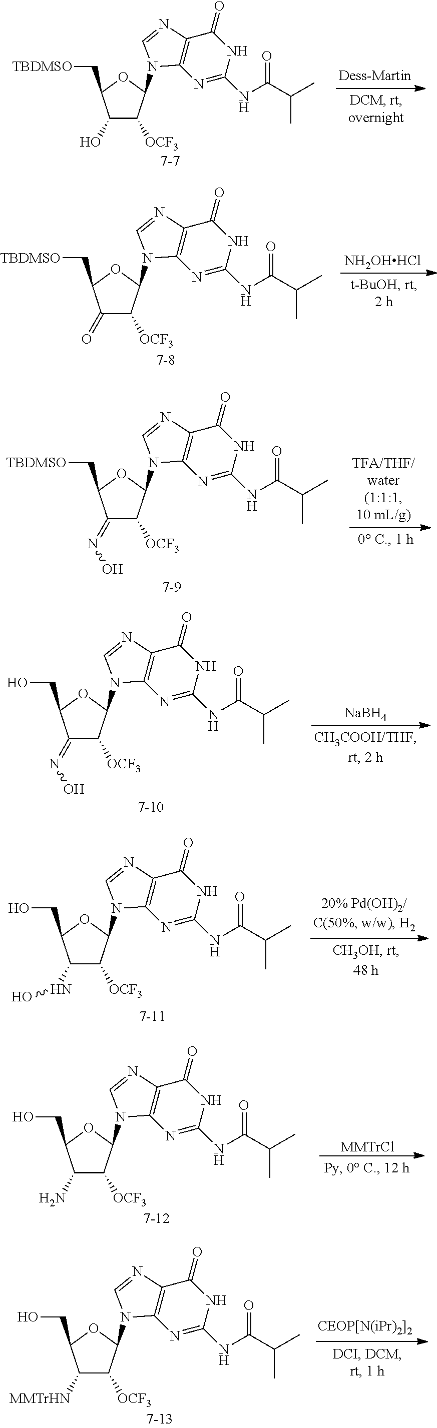

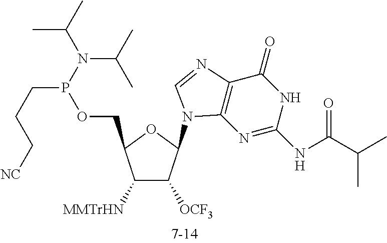

### Synthesis of 7-2

To a solution of N-[9-[(2R,3R,4S,5R)-3,4-dihydroxy-5-(hydroxymethyl)oxolan-2-yl]-6-oxo-6,9-dihydro-1H-purin-2-yl]-2-methylpropanamide (7-1; 250 g, 727.71 mmol, 1 eq.) in 2.5 L of pyridine with an inert atmosphere of nitrogen was added chloro([[chlorobis(propan-2-yl)silyl]oxy])bis(propan-2-yl)silane (330 g, 1.1 mol, 1.5 eq.) dropwise with stirring at 0° C. The resulting solution was stirred for 12 h at room temperature. The reaction was quenched by the addition of water/ice. The resulting solution was diluted with ethylacetate. The organic phase washed with water and saturated aqueous sodium chloride respectively. The organic phase was dried over anhydrous sodium sulfate, filtered, and concentrated under reduced pressure. The crude product was applied onto a silica gel column. 270 g (62%) 7-2 was obtained as a white solid. MS m/z [M+H]+ (ESI): 596.

### Synthesis of 7-3

To a solution of 7-2 (240 g, 405.12 mmol, 1 eq.) in 2 L of dichloromethane with an inert atmosphere of nitrogen was added 7-A (72 g, 482.41 mmol, 1.2 eq.), 1,8-Diazabicyclo[5.4.0]undec-7-ene (96 g, 604.82 mmol, 1.5 eq.) in order. The resulting solution was stirred for 16 h at room temperature. The resulting solution was diluted with 2 L of dichloromethane. The organic phase washed with 2N hydrochloric acid and saturated aqueous sodium chloride respectively. The organic phase was dried over anhydrous sodium sulfate, filtered, and concentrated under reduced pressure. The crude product was applied onto a silica gel column. 240 g (86.8%) 7-3 was obtained as a white solid. MS m/z [M+H]+ (ESI): 686.

### Synthesis of 7-4

To a solution of 7-3 (240 g, 350.89 mmol, 1 eq.) in acetic acid (500 mL) with an inert atmosphere of nitrogen was added acetic anhydride at room temperature. To this was added sulfuric acid (67 g, 687.74 mmol, 1.96 eq.) dropwise with stirring at 0° C. The resulting solution was stirred for 16 h at room temperature. The resulting solution was diluted with 2 L of ethylacetate. The pH value of the solution was adjusted to 7 with aqueous sodium bicarbonate. The organic phase was washed with saturated aqueous sodium chloride, dried over anhydrous sodium sulfate, filtered and concentrated under reduced pressure. The crude product was applied onto a silica gel column. 100 g (54%) 7-4 was obtained as a white solid. MS m/z [M+H]+ (ESI): 528.

### Synthesis of 7-5

To a solution of 1,3-dibromo-5,5-dimethylimidazolidine-2,4-dione (134 g, 472.5 mmol, 4.98 eq.) in 2500 mL of dichloromethane with an inert atmosphere of nitrogen, was added pyridine hydrofluoride (200 mL) dropwise with stirring at −78° C. Then a solution of 7-4 (50 g, 94.9 mmol, 1.0 eq.) in 500 mL of dichloromethane was added dropwise with stirring at −78° C. The resulting solution was stirred for 2 h at −30° C. and diluted with dichloromethane. The pH value of the solution was adjusted to 7-8 with saturated aqueous sodium bicarbonate. The resulting solution was extracted with dichloromethane. The organic phase was dried over anhydrous sodium sulfate, filtered, and concentrated under reduced pressure. The crude product was purified by Flash-Prep-HPLC. The fraction was concentrated under reduced pressure. 26.8 g (56%) of 7-5 was obtained as a light yellow solid. MS m/z [M+H]+ (ESI): 506.

### Synthesis of 7-6

To a solution of 7-5 (79.5 g, 157.35 mmol, 1 eq.) in 800 mL of methanol/pyridine/water (13:6:1) with an inert atmosphere of nitrogen was added sodium hydroxide solution (150.00 mL, 314.78 mmol, 2 eq.) dropwise with stirring at 0° C. The resulting solution was stirred for 2 hours at room temperature. The pH value of the solution was adjusted to 7 with acetic acid. The resulting solution was diluted with 1 L of ethyl acetate. The organic phase was washed with water and aqueous sodium chloride respectively. The organic phase was dried over anhydrous sodium sulfate, filtered, and concentrated under reduced pressure. The crude product was purified by Flash-Prep-HPLC. The fraction was concentrated under reduced pressure. 50 g (75.4%) of 7-6 was obtained as a light yellow solid. MS m/z [M+H]+ (ESI): 422.

### Synthesis of 7-7

To a solution of 7-6 (41 g, 97.12 mmol, 1 eq.) in N, N-Dimethylformamide (400 mL) with an inert atmosphere of nitrogen was added imidazole (1.211 g, 17.79 mmol, 1.50 eq.) at room temperature. To this was added tert-Butyldimethylsilyl chloride (9.9 g, 97.91 mmol, 1.01 eq.) with stirring at 0° C. The resulting solution was stirred overnight at room temperature. The reaction was quenched by the addition of methanol at room temperature. Then the reaction mixture was concentrated under reduced pressure. The crude product was applied onto a silica gel column. 45 g (86.5%) of 7-7 was obtained as a light yellow solid. MS m/z [M+H]+(ESI): 536. ¹H-NMR: (300 MHz, DMSO-d6) δ 12.14 (s, 1H), 11.67 (s, 1H), 8.18 (s, 1H), 6.16 (d, J=6.0 Hz, 1H), 6.05 (d, J=5.3 Hz, 1H), 5.31 (m, J=6.0, 4.8 Hz, 1H), 4.41-4.42 (m, 1H), 4.13-3.99 (m, 1H), 3.85-3.87 (m, 2H), 2.78-2.81 (m, 1H), 1.14 (d, J=6.8 Hz, 6H), 0.88 (s, 9H), 0.07 (d, J=1.6 Hz, 6H).

### Synthesis of 7-8

To a solution of 7-7 (40 g, 74.80 mmol, 1 eq.) in dichloromethane (400 mL) with an inert atmosphere of nitrogen, was added Dess-Martin (41.1 g, 96.97 mmol, 1.30 eq.) at room temperature. The resulting solution was stirred overnight at room temperature. The solids were filtered out. The filtrate was used in next step without further purification.

### Synthesis of 7-9

To a solution of 7-8 (35 g crude in tert-Butanol 1 L and dichloromethane 1 L) with an inert atmosphere of nitrogen was added hydroxylamine hydrochloride (19 g, 271.94 mmol, 3.00 eq.). The resulting solution was stirred at room temperature for 2 h. The resulting solution was diluted with 1 L of dichloromethane. The organic phase was washed with water and aqueous sodium chloride respectively. The organic phase was dried over anhydrous sodium sulfate, filtered and concentrated under reduced pressure. The residue was purified by Flash-Prep-HPLC. The fraction was concentrated under reduced pressure. 30 g (60.3%) of 7-10 was obtained as a white solid. MS m/z [M+H]+ (ESI): 506.

### Synthesis of 7-10

To a solution of 7-9 (25 g, 46.06 mmol, 1 eq.) in 250 mL of tetrahydrofuran/water/trifluoroacetic acid (1:1:1) with an inert atmosphere of nitrogen was stirred at 0° C. for 1 h. The resulting solution was concentrated under reduced pressure. The residue was purified by Flash-Prep-HPLC. The fraction was concentrated under reduced pressure. 12.5 g (63.1%) of 7-10 was obtained as a white solid. MS m/z [M+H]+ (ESI): 435.

### Synthesis of 7-11

To a solution of acetic acid/tetrahydrofuran (3.3/1, 120 mL) with an inert atmosphere of nitrogen was added sodium borohydride (3.5 g, 92.10 mmol, 4.00 eq.) at 0° C. for several batches. The resulting solution was stirred at 0° C. for 10 min. To this was added 7-10 (10 g, 23.00 mmol, 1 eq.) in acetic acid (60 mL) dropwise with stirring at 0° C. Then the resulting solution was stirred at room temperature for 2 h. The reaction was quenched by water. The resulting solution was concentrated under reduced pressure. The residue was purified by Flash-Prep-HPLC. The fraction was concentrated under reduced pressure. 5.0 g (49.8%) of 7-11 was obtained as a white solid. MS m/z [M+H]+ (E51):437. H-NMR: (400 MHz, DMSO-d6) δ 12.10 (s, 1H), 11.75 (s, 1H), 8.34 (s, 1H), 7.72 (s, 1H), 6.26 (d, J=6.9 Hz, 2H), 5.38-5.21 (m, 2H), 4.27 (q, J=3.4 Hz, 1H), 3.67-3.88 (m, 2H), 3.61 (d, J=12.0 Hz, 1H), 2.76-2.79 (m, 1H), 1.13-1.10 (m, 6H).

### Synthesis of 7-12

To a solution of 7-11 (4.4 g, 10.19 mmol, 1 eq.) in methanol (44 mL) was added 20% Palladium hydroxide on activated carbon (4.3 g, 30.57 mmol, 3.00 eq.) at room temperature. The flask was evacuated and flushed five times with hydrogen. The resulting solution was stirred at room temperature for 48 h. The solids were filtered out. The resulting solution was concentrated until no residual solvent left under reduced pressure. The residue was purified by Flash-Prep-HPLC. The fraction was concentrated under reduced pressure. 4.0 g (93.4%) of 7-12 was obtained as a white solid. MS m/z [M+H]+ (ESI): 421.

### Synthesis of 7-13

To a solution of 7-12 (3.2 g, 7.61 mmol, 1 eq.) in pyridine (30 mL) with an inert atmosphere of nitrogen was added 4-methoxytriphenylchloromethane (2.34 g, 7.61 mmol, 1.00 eq.) with stirring at 0° C. The resulting solution was stirred at 0° C. for 12 h. The reaction was quenched by 10 mL of methanol and the resulting solution was concentrated until no residual solvent left under reduced pressure. The residue was diluted with 300 mL of dichloromethane. The organic phase was washed with 2×100 mL of aqueous sodium chloride, dried over anhydrous sodium sulfate, filtered and concentrated until no residual solvent left under reduced pressure. The residue was purified by Flash-Prep-HPLC. The fraction was concentrated under reduced pressure. 3.2 g (51.2%) of 7-13 was obtained as a white solid. MS m/z[M+Na]⁺ (ESI): 693. ¹H-NMR: (DMSO-d6, 300 MHz, ppm): δ12.20 (s, 1H), 11.59 (s, 1H), 8.20 (s, 1H), 7.51 (d, J=7.5 Hz, 4H), 7.38 (d, J=8.7 Hz, 2H), 7.25-7.30 (t, J=7.5 Hz, 4H), 7.16-7.21 (t, J=7.2 Hz, 2H), 6.83 (d, J=9.0 Hz, 2H), 6.23 (d, J=5.4 Hz, 1H), 5.20-5.23 (t, J=4.7 Hz, 1H), 4.37-4.40 (t, J=5.4 Hz, 1H), 3.70 (s, 3H), 3.51-3.56 (m, 1H), 3.38-3.50 (m, 3H), 3.20-3.32 (m, 1H), 2.80-2.89 (m, 1H), 1.10-1.14 (t, J=6.0 Hz, 6H). F-NMR: (DMSO, 300 MHz, ppm): −56.35.

### Synthesis of 7-14

To a solution of 7-13 (3.2 g, 4.62 mmol, 1 eq.) in dichloromethane (32 mL) with an inert atmosphere of nitrogen, was added 3-([bis[bis(propan-2-yl)amino]phosphanyl]oxy)propanenitrile (1.95 g, 6.47 mmol, 1.40 eq.) and 1H-imidazole-4,5-dicarbonitrile (0.6 g, 5.08 mmol, 1.10 eq.) in order. The resulting solution was stirred for 1 hour at room temperature. The resulting solution was diluted with dichloromethane. The organic phase was washed with aqueous sodium bicarbonate and aqueous sodium chloride respectively. The organic phase was dried over anhydrous sodium sulfate and concentrated under reduced pressure. The residue was purified by Flash-Prep-HPLC. The fractions were diluted with dichloromethane and dried over anhydrous sodium sulfate. The solid was filtered out. The filtrate was concentrated under reduced pressure. 3.0 g (72.7%) of 7-14 was obtained as a white solid. MS m/z [M+H]+ (ESI): 893. H-NMR: (DMSO-d6, 400 MHz, ppm):δ 12.18 (s, 1H), 11.55 (d, J=72 Hz, 1H), 8.02 (d, J=24.0 Hz, 1H), 7.48-7.53 (m, 4H), 7.17-7.40 (m, 8H), 6.82 (dd, J=8.8 Hz, 2H), 6.21 (dd, J=4.0 Hz, 1H), 3.88-4.77 (m, 1H), 3.51-3.72 (m, 7H), 3.24-3.45 (m, 5H), 2.72-2.85 (m, 3H), 1.21-1.23 (m, 1H), 1.09-1.15 (m, 12H), 0.95-1.04 (m, 5H). F-NMR: (DMSO, 400 MHz, ppm): −55.40, −56.81. P-NMR: (DMSO, 400 MHz, ppm): 148.50, 148.16.

### Example 8

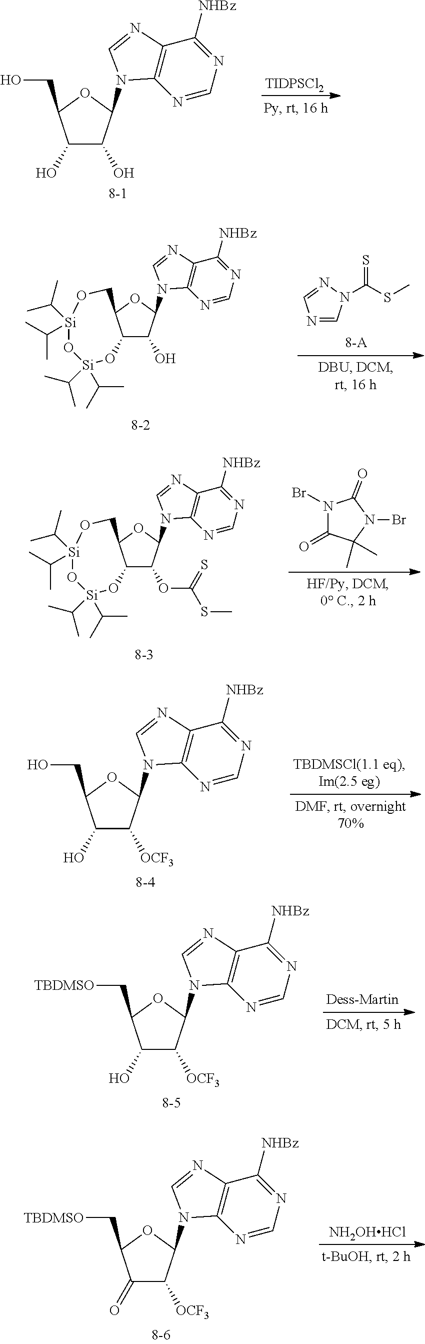

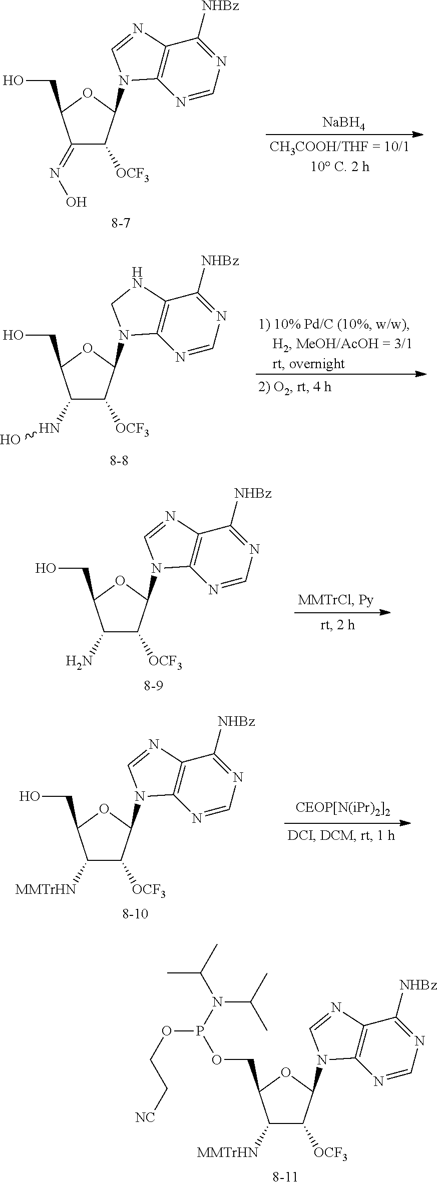

### Synthesis of 8-2

To a solution of N-[9-[(2R,3R,4S,5R)-3,4-dihydroxy-5-(hydroxymethyl)oxolan-2-yl]-9H-purin-6-yl]benzamide (8-1; 400 g, 1077 mmol, 1 eq.) in 12000 mL of pyridine with an inert atmosphere of nitrogen was added 1,3-Dichloro-1,1,3,3-tetraisopropyldisiloxane (374 g, 1186 mmol, 1.10 eq.) dropwise with stirring at 0° C. The resulting solution was stirred for 16 h at room temperature and concentrated under reduced pressure. The residue was diluted with 10000 mL of dichloromethane and washed with saturated aqueous sodium chloride. The organic phase was dried over anhydrous sodium sulfate, filtered, concentrated under reduced pressure. The crude product was applied onto a silica gel column. 520 g (80%) of 8-2 was obtained as a white solid. MS m/z [M+H]+ (ESI): 614.

### Synthesis of 8-3

To a solution of 8-2 (110 g, 179 mmol, 1 eq.) in 1100 mL of dichloromethane with an inert atmosphere of nitrogen, was added 8-A (51.3 g, 323 mmol, 1.80 eq.) at room temperature. Then 1,8-Diazabicyclo [5.4.0] undec-7-ene (35 g, 233 mmol, 1.3 eq.) dropwise was added with stirring at 0° C. The resulting solution was stirred at room temperature for 16 h and diluted with 2000 mL of dichloromethane. The organic layer phase was washed with 5% hydrochloric acid, saturated aqueous sodium bicarbonate, and saturated aqueous sodium chloride respectively. The organic phase was dried over anhydrous sodium sulfate, filtered, concentrated under reduced pressure. The residue was applied onto a silica gel column. 85 g (65%) of 8-3 was obtained as a white solid. MS m/z [M+H]+ (ESI): 704.

### Synthesis of 8-4

To a solution of 1,3-Dibromo-5,5-dimethylhydantoin (65.0 g, 227.33 mmol, 8.00 eq.) in 400 mL of dichloromethane with an inert atmosphere of nitrogen was added pyridine hydrofluoride (130 mL, 1442.90 mmol, 50.79 eq.) dropwise with stirring at −20° C. Then 8-3 (20 g, 28.41 mmol, 1 eq.) in dichloromethane was added dropwise with stirring at −20° C. The resulting solution was stirred at 0° C. for 2 hours and diluted with dichloromethane. The pH value of the solution was adjusted to 7 with saturated aqueous sodium bicarbonate. The organic phase was washed with saturated sodium chloride aq. The organic layer phase was dried over anhydrous sodium sulfate, filtered, concentrated under reduced pressure. The residue was purified by Flash-Prep-HPLC. The fraction was concentrated under reduced pressure. 9.7 g (24%) of 8-4 was obtained as a white solid. MS m/z [M+H]+ (ESI): 440.

### Synthesis of 8-5

To a solution of 8-4 (10 g, 22.8 mmol, 1 eq.) in 100 mL N, N-Dimethylformamide with an inert atmosphere of nitrogen was added imidazole (3.87 g, 56.9 mmol, 2.50 eq.) at room temperature. Then tert-butyldimethylsilyl chloride (3.77 g, 25 mmol, 1.10 eq.) was added with stirring at 0° C. The resulting solution was stirred at room temperature overnight and quenched by the addition of 50 mL of methanol at room temperature. The reaction mixture was concentrated under reduced pressure. The residue was applied onto a silica gel column. 8.8 g (70%) of 8-5 was obtained as a white solid. MS m/z [M+H]+ (ESI): 554

### Synthesis of 8-6

To a solution of 8-5 (20 g, 36 mmol, 1.0 eq.) in 200 mL of dichloromethane with an inert atmosphere of nitrogen was added Dess-Martin (3.06 g, 72 mmol, 2.0 eq.) at room temperature. The resulting solution was stirred for 2 h at room temperature and diluted with tert-butanol. The solids were filtered out. The filtrate was used in the next step directly without further purification (containing ˜18 g 8-6). MS m/z [M+H]+ (ESI): 554

### Synthesis of 8-7

To a solution of 8-6 (18 g crude in 600 mL of tert-butanol and 200 mL of dichloromethane, from last step) with an inert atmosphere of nitrogen was added Hydroxylamine hydrochloride (6.5 g, 93 mmol, 2.91 eq.) with stirring at room temperature. The resulting solution was stirred at room temperature for 2 h and concentrated until no residual solvent left under reduced pressure. The residue was purified by Flash-Prep-HPLC. The fractions were diluted with dichloromethane and dried over anhydrous sodium sulfate. The solid was filtered out. The filtrate was concentrated under reduced pressure. 7.2 g (48% over two steps) of 8-7 was obtained as a purple solid. MS m/z [M+H]⁺ (ESI): 453.

### Synthesis of 8-8

To a solution of acetic acid/tetrahydrofuran (10/1, 150 mL) with an inert atmosphere of nitrogen was added sodium borohydride (6.2 g, 164 mmol, 10 eq.) at 0° C. for several batches. The resulting solution was stirred at 0° C. for 20 min. To this was added 8-7 (7.2 g, 16.4 mmol, 1 eq.) in 7 mL acetic acid dropwise with stirring at 0° C. and resulting solution was stirred at 0° C. for 2 h. The resulting solution was quenched by the addition of water at room temperature. The reaction was concentrated until no residual solvent left under reduced pressure. The residue was purified by Flash-Prep-HP LC. The fraction was concentrated under reduced pressure. 5.2 g (70%) of 8-8 was obtained as yellow solid. MS m/z [M+H]⁺ (ESI): 457. ¹H NMR (CD₃OD, 300 Hz, ppm) δ 8.02-7.99 (d, J=9.0 Hz, 2H), 7.88 (s, 1H) 7.65-7.60 (t, J=7.5 Hz, 1H), 7.56-7.51 (t, J=7.5 Hz 2H), 6.04-6.01 (d, J=9.0 Hz, 1H), 5.33-5.32 (d, J=3.0 Hz, 1H), 5.22-5.18 (m, 2H), 4.24-4.21 (m, 1H), 3.84-3.80 (m, 1H), 3.74-3.80 (m, 1H) 3.70-3.65 (m, 1H). F-NMR(CD₃OD, 300 Hz, ppm) δ −60.61.

### Synthesis of 8-9

To a solution of 8-8 (5.2 g, 11.4 mmol 1.00 eq.) in 50 mL of methanol/aqueous acetic acid (3:1, v/v, 10 mL/g) was added 10% palladium on activated carbon (0.5 g) at room temperature. The flask was evacuated and flushed five times with hydrogen. The resulting solution was stirred at room temperature overnight. The flask was evacuated and flushed five times with oxygen. The resulting solution was stirred at room temperature for 4 h and the solids were filtered out. The pH value of the solution was adjusted to 7 with ammonium hydroxide at 0° C. and then concentrated under reduced pressure. The residue was purified by Flash-Prep-HPLC. The fraction was concentrated under reduced pressure. 3.53 g (70%) of 8-9 was obtained as a white solid. MS m/z [M+H]+ (ESI): 439. ¹H NMR (300 MHz, DMSO-d₆) δ 11.26 (s, 1H), 8.78 (s, 2H), 8.06-8.04 (d, J=6.0 Hz, 2H), 7.65-7.61 (t, J=6.0 Hz, 1H), 7.58-7.54 (t, J=6.0 Hz, 2H), 6.42 (s, 1H), 5.32 (s, 1H), 5.20-5.17 (t, J=4.5 Hz, 1H), 4.12-3.80 (m, 3H), 3.65 (s, 1H), 1.36 (s, 2H). F-NMR (300 MHz, DMSO-d₆) δ −56.90.

### Synthesis of 8-10

To a solution of 8-9 (3.53 g, 8.06 mmol, 1 eq.) in 30 mL of pyridine with an inert atmosphere of nitrogen was added 4-methoxytriphenylchloromethane (2.9 g, 9.68 mmol, 1.20 eq.) with stirring at 0° C. The resulting solution was stirred at room temperature for 2 h. The reaction was quenched by methanol and the resulting solution was concentrated until no residual solvent left under reduced pressure. The residue was diluted with dichloromethane and washed with brine. The organic layer phase was dried over anhydrous sodium sulfate, filtered, concentrated until no residual solvent left under reduced pressure. The residue was purified by Flash-Prep-HPLC. The fractions were diluted with dichloromethane. The organic phase was dried over anhydrous sodium sulfate, filtered, and concentrated under reduced pressure. 3 g (75%) of 8-10 was obtained as a white solid. MS m/z [M+Na]⁺ (ESI): 711. ¹H NMR (DMSO-d₆, 300 Hz, ppm) δ 11.21 (s, 1H), 8.71 (s, 2H), 8.07-8.05 (d, J=6.0 Hz, 2H), 7.65-7.45 (m, 7H), 7.34-7.15 (m, 9H), 6.78-6.76 (d, J=6.0 Hz, 2H), 6.40 (s, 1H), 5.22-5.19 (t, J=4.5 Hz, 1H), 3.98-3.95 (t, J=4.5 Hz, 1H), 3.82-3.80 (d, J=6.0 Hz, 1H), 3.74-3.56 (m, 5H), 3.17-3.13 (d, J=12.0 Hz, 1H). F NMR (DMSO-d₆, 300 Hz, ppm) δ −55.46.

### Synthesis of 8-11

To a solution of 8-10 (3 g, 4.22 mmol, 1 eq.) in 50 mL of dichloromethane with an inert atmosphere of nitrogen was added bis(diisopropylamino) (2-cyanoethoxy) phosphine (1.8 g, 5.91 mmol, 1.4 eq.). To this was added 4, 5-Dicyanoimidazole (0.5 g, 4.64 mmol, 1.1 eq.) at room temperature. The resulting solution was stirred for 1 h at room temperature and diluted with 200 mL of dichloromethane, washed with saturated aqueous sodium bicarbonate and saturated aqueous sodium chloride respectively. The organic phase was dried over anhydrous sodium sulfate, filtered, and concentrated until no residual solvent left under reduced pressure. The crude product was purified by Flash-Prep-HPLC. The fractions were diluted with dichloromethane. The organic phase was dried over anhydrous sodium sulfate, filtered, and concentrated under reduced pressure. The product, which was dried for 8 h under, reduced pressure at 25° C. 2.86 g (70%) of 8-11 was obtained as a white solid. MS m/z [M+Na]⁺ (ESI): 911. ¹H NMR (DMSO-d₆, 300 Hz, ppm) δ 11.21 (s, 1H), 8.67-8.66 (d, J=3.0 Hz, 1H), 8.52-8.50 (d, J=6.0 Hz, 1H), 8.07-8.04 (d, J=12.0 Hz, 2H), 7.65-7.62 (m, 1H), 7.58-7.41 (m, 6H), 7.38-7.13 (m, 8H), 6.85-6.75 (m, 2H), 6.72-6.34 (m, 1H), 4.62-4.15 (m, 1H), 3.87-3.82 (m, 2H), 3.70-3.63 (m, 2H), 3.63-3.33 (m, 6H), 3.16-3.12 (d, J=12.0 Hz, 1H), 2.73-2.51 (m, 2H), 1.23 (s, 1H), 1.10-0.95 (m, 12H). P-NMR (DMSO-d₆, 300 Hz, ppm) δ 147.86, 147.46. F-NMR (DMSO-d₆, 300 Hz, ppm) δ −55.19, −56.89.

### Example 9

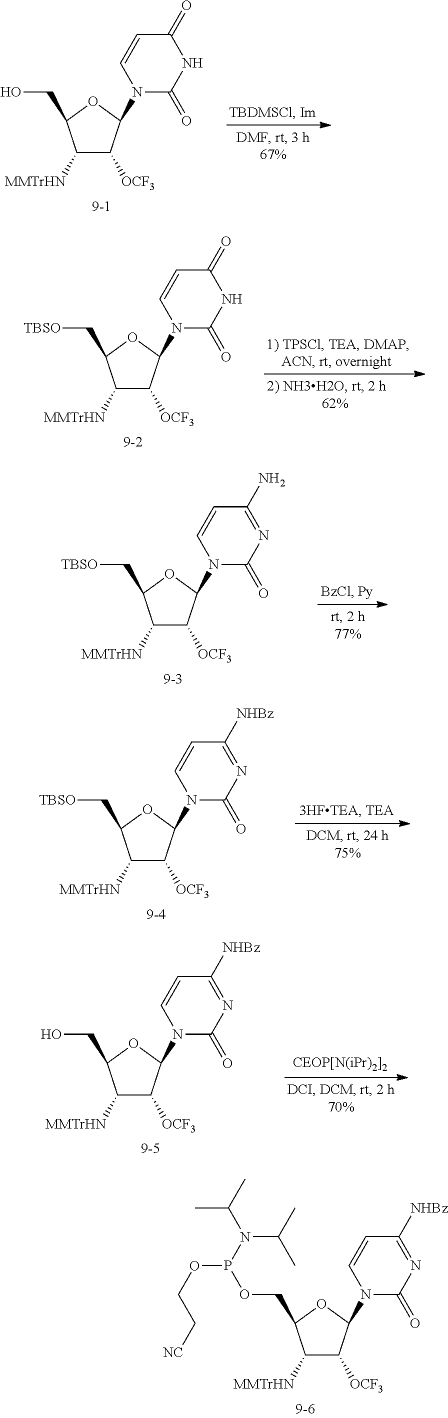

### Synthesis of 9-2

To a solution of 9-1 (7.6 g, 13.01 mmol, 1.00 eq.) in 120 mL of N, N-Dimethylformamide with an inert atmosphere of nitrogen, was added imidazole (2.22 g, 32.56 mmol, 2.50 eq.) at room temperature. To this was added tert-Butyldimethylsilyl chloride (2.75 g, 18.24 mmol, 1.40 eq.) with stirring at 0° C. The resulting solution was stirred at room temperature for 3 h and then quenched by the addition of 10 mL of methanol at room temperature. The resulting solution was concentrated under reduced pressure. The residue was purified by Flash-Prep-HPLC. 6 g (67%) of 9-2 was obtained as a white solid. MS m/z [M+H]+ (ESI): 698.

### Synthesis of 9-3

To a solution of 9-2 (6 g, 8.61 mmol, 1.00 eq.) in 60 mL of acetonitrile with an inert atmosphere of nitrogen, was added triethylamine (2.7 g, 25.82 mmol, 3.00 eq.), 4-Dimethylaminopyridine (3.15 g, 25.82 mmol, 3.00 eq.) and 2, 4, 6-Triisopropylbenzenesulfonyl chloride (7.8 g, 25.82 mmol, 3.00 eq.) in order at room temperature. The resulting solution was stirred overnight at room temperature. Then ammonium hydroxide (20 mL) was added with stirring at room temperature and the resulting mixture was stirred at room temperature for 2 h. The reaction mixture was concentrated under reduced pressure. The residue was purified by Flash-Prep-HPLC. 3.7 g (62%) of 9-3 was obtained as a white solid. MS m/z [M+H]+ (ESI): 697.

### Synthesis of 9-4

To a solution of 9-3 (3.7 g, 5.31 mmol, 1.00 eq.) in 37 mL of pyridine with an inert atmosphere of nitrogen, was added benzoyl chloride (1.1 g, 7.83 mmol, 1.50 eq.) dropwise with stirring at room temperature. The resulting solution was stirred at room temperature for 2 h. The reaction was then quenched by the addition of 10 mL of methanol at room temperature and diluted with 200 mL dichloromethane. The resulting solution was washed with brine, dried over anhydrous sodium sulfate, filtered, concentrated under reduced pressure. The residue was purified by Flash-Prep-HPLC. 3.3 g (77%) of 9-4 was obtained as a white solid. MS m/z [M+H]+ (ESI): 801.

### Synthesis of 9-5

To a solution of 9-4 (3.3 g, 4.12 mmol, 1.00 eq.) and triethylamine (2.5 g, 24.72 mmol, 6.00 eq.) in 33 mL of dichloromethane with an inert atmosphere of nitrogen, was added triethylamine trihydrofluoride (1.7 g, 10.55 mmol, 2.56 eq.). The resulting solution was stirred at room temperature for 24 h. The reaction mixture was diluted with dichloromethane and washed with saturated sodium bicarbonate and brine respectively. The organic layer was dried over anhydrous sodium sulfate, filtered, concentrated under reduced pressure. The residue was purified by Flash-Prep-HPLC. 2.1 g (70%) of 9-5 was obtained as a white solid. MS m/z [M+H]+ (ESI): 687. ¹H-NMR (DMSO-d₆, 400 Hz, ppm): δ 11.29 (s, 111), 8.52-8.51 (d, J=7.2 Hz, 1H), 8.01-7.99 (d, J=7.2 Hz, 2H), 7.64-7.61 (m, 1H), 7.53-7.44 (m, 6H), 7.32-7.16 (m, 9H), 6.81-6.80 (d, J=4.4 Hz, 2H), 5.92 (s, 1H), 5.27-5.26 (t, J=3.6 Hz, 1H), 3.92-3.72 (m, 6H), 3.34-3.40 (m, 2H), 2.93-2.91 (d, J=10.0 Hz, 1H). F-NMR (DMSO-d₆, 400 Hz, ppm): −54.96

### Synthesis of 9-6

To a solution of 9-5 (2.1 g, 3.06 mmol, 1.00 eq.) in 30 mL of dichloromethane with an inert atmosphere of nitrogen was added 1H-imidazole-4, 5-dicarbonitrile (0.4 g, 3.36 mmol, 1.10 eq.) and Bis (diisopropylamino) (2-cyanoethoxy) phosphine (1.3 g, 4.28 mmol, 1.40 eq.) in order at room temperature. The resulting solution was stirred at room temperature for 2 h. The reaction mixture was diluted with dichloromethane and washed with saturated sodium bicarbonate and brine respectively. The organic layer was dried over anhydrous sodium sulfate, filtered, concentrated until no residual solvent left under reduced pressure. The residue was purified by Flash-Prep-HPLC. The fractions were diluted with dichloromethane and dried over anhydrous sodium sulfate. The solid was filtered out. The filtrate was concentrated until no residual solvent left under reduced pressure, obtained the product, which was dried for 8 h under reduced pressure at 25° C. 1.91 g (70%) of 9-6 was obtained as a white solid. MS m/z [M+H]+ (ESI): 887. ¹H-NMR (DMSO-d₆, 400 Hz, ppm): δ 11.32 (s, 1H), 8.33-8.02 (m, 1H), 8.00-7.99 (d, J=1.6 Hz, 2H), 7.63-7.50 (m, 7H), 7.47-7.17 (m, 9H), 6.83-6.79 (t, J=8.2 Hz, 2H), 6.08-5.85 (m, 1H), 4.26-4.01 (m, 1H), 3.99-3.89 (m, 1H), 3.78-3.57 (m, 5H), 3.57-3.54 (m, 3H), 3.32-3.31 (m, 2H), 2.91-2.90 (m, 1H), 2.78-2.74 (m, 2H), 1.15-1.14 (d, J=3.2 Hz, 6H), 1.10-1.04 (m, 6H) P-NMR (DMSO-d₆, 400 Hz, ppm): 148.34, 147.68. F-NMR (DMSO-d₆, 400 Hz, ppm): −54.73, −55.42.

### Example 10

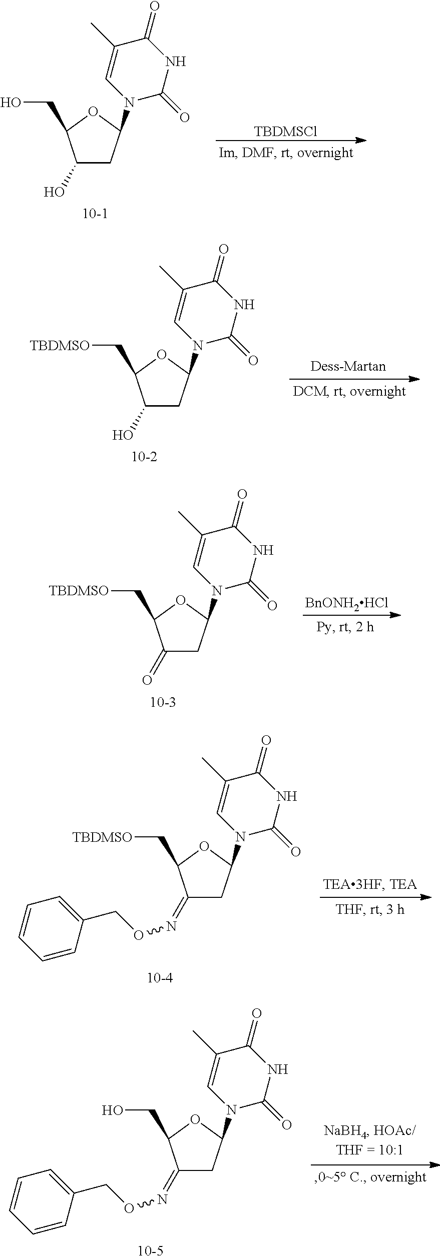

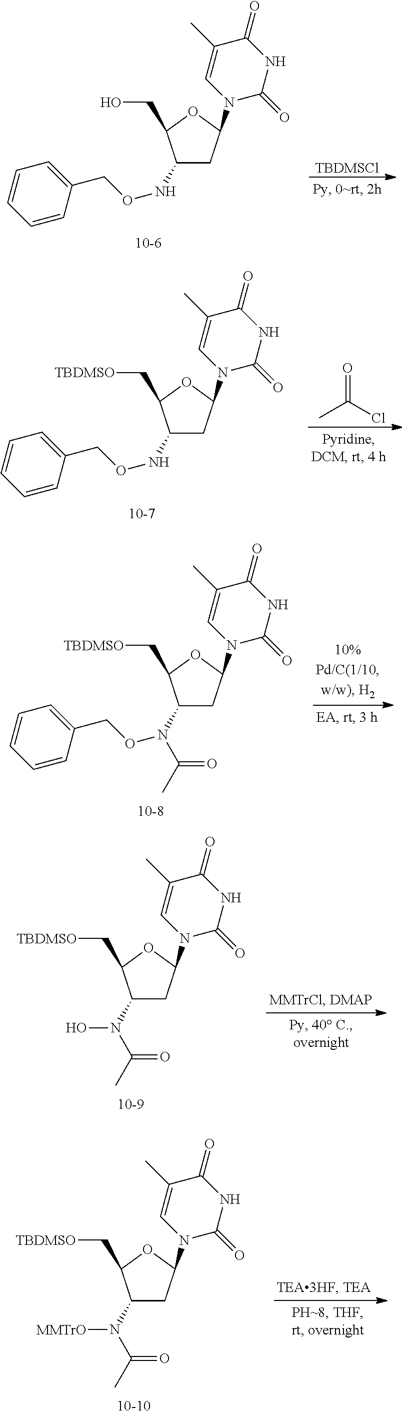

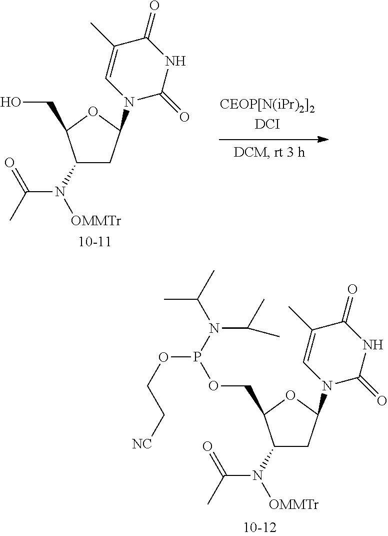

### Synthesis of 10-2

To a solution of 1-[(2R,4S,5R)-4-hydroxy-5-(hydroxymethyl)oxolan-2-yl]-5-methyl-1,2,3,4-tetrahydropyrimidine-2,4-dione (100 g, 0.41 mol, 1.0 eq) in 2000 mL of dimethyl formamide was added imidazole (56.3 g, 0.84 mol, 2 equiv) at 25° C. Then the tert-Butyldimethylsilyl chloride (64.2 g, 0.426 mol, 1.04 equiv) was added at 0° C. The resulting solution was stirred for 16 h at 25° C. and quenched by 100 mL of methanol at 0° C. The resulting solution was concentrated under reduced pressure. The residue was dissolved in 4 L of dichloromethane, washed with 2×2 L of saturated aqueous sodium bicarbonate and 1×2 L of saturated aqueous sodium chloride respectively. The organic phase was dried over anhydrous sodium sulfate, filtered and concentrated under reduced pressure. The crude product was applied onto a silica gel column. 120 g (81%) of 10-2 was obtained as a white solid. MS m/z [M+H]+ (ESI): 357.

### Synthesis of 10-3

To a solution of 10-2 (120 g, 0.34 mol, 1.0 eq) in 1200 mL of dichloromethane was added Dess-Martin (171.5 g, 0.40 mol, 1.20 equiv) at 25° C. Then the resulting solution was stirred for 16 h at 25° C. The residue was dissolved in 3 L of dichloromethane, washed with 5×1 L of saturated aqueous potassium bicarbonate and 1×1 L of saturated aqueous sodium chloride respectively. The organic phase was dried over anhydrous sodium sulfate, filtered and concentrated under reduced pressure. 110 g (crude) 10-3 was obtained as an off-white solid. MS m/z [M+H]+ (ESI): 355.

### Synthesis of 10-4

To a solution of 10-3 (75 g, 0.21 mmol, 1.0 eq) in 750 ml of pyridine with an inert atmosphere of nitrogen was added O-benzylhydroxylamine hydrochloride (100.5 g, 0.63 mmol, 3.0 equiv) and potassium acetate (15.96 g, 0.84 mmol, 4.0 eq) in order at 0° C. The resulting solution was stirred for 2 h at 25° C. and diluted with 2 L of dichloromethane, washed with 2×1 L of saturated aqueous sodium bicarbonate and 1×1 L of saturated aqueous sodium chloride respectively. The organic phase was dried over anhydrous sodium sulfate, filtered and concentrated under reduced pressure. The residue was applied onto a silica gel column. 70 g (74%) of 10-4 was obtained as a white solid. MS m/z [M+H]+ (ESI): 460.

### Synthesis of 10-5

To a solution of 10-4 (75 g, 0.168 mol, 1.00 equiv) in 750 ml of dichloromethane with an inert atmosphere of nitrogen, was added triethylamine (102 g, 1.01 mol, 6 equiv) and triethylamine trihydrofluoride (53.1 g, 0.33 mol, 2 equiv) in order at room temperature. The resulting solution was stirred for 16 h at 25° C. and diluted with 2 L of dichloromethane, washed with 2×1 L of saturated aqueous sodium bicarbonate and 1×1 L of saturated aqueous sodium chloride respectively. The organic phase was dried over anhydrous sodium sulfate, filtered and concentrated under reduced pressure. The crude product was applied onto a silica gel column. 35 g (62.5%) of 10-5 was obtained as a white solid. MS m/z [M+H]+ (ESI): 346.

### Synthesis of 10-6

To a solution of 10-5 (20 g, 57.64 mmol, 1.0 eq) in 200 mL of acetic acid/tetrahydrofuran (10:1) with an inert atmosphere of nitrogen was added sodium borohydride (6.53 g, 172.9 mmol, 3.0 eq) at 0° C. The resulting solution was stirred for 16 h at 0° C. and quenched by 50 mL of methanol at 0° C. The resulting solution was concentrated under reduced pressure. The residue was purified by Flash-Prep-HPLC. The fractions were diluted with 2000 mL of dichloromethane and the organic phase was dried over anhydrous sodium sulfate. The solid was filtered out. The filtrate was concentrated under reduced pressure. 10.1 g (50%) of 10-6 was obtained as a white solid. MS m/z [M+H₂O]+ (ESI): 365.

### Synthesis of 10-7

To a solution of 10-6 (24 g, 0.69 mol, 1.0 eq) in 220 mL of dry pyridine was added tert-Butyldimethylsilyl chloride (12 g, 0.80 mol, 1.15 equiv) at room temperature. The resulting solution was stirred for 3 h at 25° C. and quenched by 100 mL of methanol at 0° C. The resulting solution was concentrated under reduced pressure. The residue was dissolved in 1 L of dichloromethane, washed with 2×500 mL of saturated aqueous sodium bicarbonate and 1×500 mL of saturated aqueous sodium chloride respectively. The organic phase was dried over anhydrous sodium sulfate, filtered and concentrated under reduced pressure. The crude product was applied onto a silica gel column. 26.0 g (81%) of 10-7 was obtained as a white solid. MS m/z [M+H]+ (ESI): 462. 1H NMR (400 MHz, DMSO-d₆) δ 11.29 (s, 1H), 7.49 (d, J=1.4 Hz, 1H), 7.37-7.25 (m, 5H), 6.90 (d, J=5.6 Hz, 1H), 6.13 (m, 1H), 4.65 (s, 2H), 3.89 (m, 1H), 3.83 (m, 1H), 3.70 (m, 1H), 3.59 (s, 1H), 2.16 (m, 1H), 2.05-1.95 (m, 1H), 1.78 (d, J=1.2 Hz, 3H), 0.88 (s, 9H), 0.06 (d, J=2.3 Hz, 6H).

### Synthesis of 10-8

To a solution of 10-7 (18 g, 39.04 mmol, 1.0 eq) in 180 ml of dichloromethane with an inert atmosphere of nitrogen, was added pyridine (15.6 g, 195 mmol, 5 eq) and acetyl chloride (3.37 g, 42.9 mmol, 1.1 eq) in order at 0° C. The resulting solution was stirred for 2 h 25° C. and diluted with 1000 mL of dichloromethane. The organic phase washed with 2×100 mL of water and 1×100 mL of saturated aqueous sodium chloride respectively. The organic phase was dried over anhydrous sodium sulfate, filtered and concentrated under reduced pressure. The residue was purified by Flash-Prep-HPLC. The fractions were diluted with 2000 mL of dichloromethane and the organic phase dried over anhydrous sodium sulfate. The solid was filtered out. The filtrate was concentrated under reduced pressure. 16.0 g (81%) of 10-8 was obtained as a white solid. MS m/z [M+H]+ (ESI): 504.

### Synthesis of 10-9

To a solution of 10-8 (16 g, 39.7 mmol, 1.00 equiv) in the 160 ml of ethyl acetate with an inert atmosphere of nitrogen was added 10% Palladium on activated carbon (1.6 g, 1.42 mmol, 0.71 equiv). The flask was evacuated and flushed five times with hydrogen. The resulting solution was stirred for 3 h at room temperature. The solids were filtered out and the filtrate was concentrated under vacuum. The crude product was applied onto a silica gel column. 10 g (51%) of 10-9 was obtained as a yellow solid. MS m/z [M+H]+ (ESI): 414. 1H NMR (300 MHz, DMSO-d₆) δ 11.36 (s, 1H), 10.05 (s, 1H), 7.52 (d, J=1.3 Hz, 1H), 6.19 (t, J=7.0 Hz, 1H), 5.08 (s, 1H), 4.03 (m, 1H), 3.88-3.77 (m, 1H), 3.77-3.68 (m, 1H), 2.31 (m, 1H), 2.19-2.04 (m, 1H), 2.07 (m, 3H), 1.78 (d, J=1.1 Hz, 3H), 0.89 (s, 9H), 0.08 (s, 6H).

### Synthesis of 10-10

To a solution of 10-9 (10 g, 24.2 mmol, 1.00 equiv) in the 70 ml of pyridine with an inert atmosphere of nitrogen, was added 4-Dimethylaminopyridine (2.95 g, 24.2 mmol, 1 equiv) and 4-Methoxytriphenylchloromethane (30 g, 100 mmol, 4.00 equiv) in order at room temperature. The resulting solution was stirred overnight at 40° C. and quenched by 30 mL of methanol at 0° C. The resulting solution was concentrated under reduced pressure. The residue was dissolved in 500 mL of dichloromethane, washed with 2×200 mL of saturated aqueous sodium bicarbonate and 1×200 mL of saturated aqueous sodium chloride respectively. The organic phase was dried over anhydrous sodium sulfate, filtered and concentrated under reduced pressure. The crude product was applied onto a silica gel column. 6 g (42%) of 10-10 was obtained as a white solid. MS m/z [M+H]+ (ESI): 686.

### Synthesis of 10-11

To a solution of 10-10 (6 g, 8.76 mmol, 1.00 equiv) in 60 ml of tetrahydrofuran with an inert atmosphere of nitrogen, was added triethylamine (7.8 g, 52.6 mmol, 6 equiv) and triethylamine trihydrofluoride (2.8 g, 17.5 mmol, 2 equiv) in order at room temperature. The resulting solution was stirred overnight at 25° C. and diluted with 300 mL of dichloromethane, washed with 2×100 mL of saturated aqueous sodium bicarbonate and 1×100 mL of saturated aqueous sodium chloride respectively. The organic phase was dried over anhydrous sodium sulfate, filtered and concentrated under reduced pressure. The residue was purified by Flash-Prep-HPLC. The fractions was diluted with 1000 mL of dichloromethane and dried over anhydrous sodium sulfate. The solid was filtered out. The filtrate was concentrated under reduced pressure. 3 g (60%) of 10-11 was obtained as a white solid. MS m/z [M+Na]+ (ESI): 594. 1H NMR (400 MHz, DMSO-d₆) δ 11.20 (d, J=2.3 Hz, 1H), 7.56 (s, 1H), 7.34 (m, 10H), 7.22-7.17 (m, 2H), 6.99-6.92 (m, 2H), 6.17 (m, 1H), 5.02 (m, 1H), 4.28-4.07 (m, 2H), 3.76 (d, J=2.4 Hz, 3H), 3.61 (d, J=11.9 Hz, 1H), 2.07 (m, 2H), 1.86 (m, 4H), 1.62 (m, 3H)

### Synthesis of 10-12

To a solution of 10-11 (500 mg, 0.875 mmol, 1.0 eq) in 5 mL of dichloromethane with an inert atmosphere of nitrogen, was added bis (diisopropylamino) (2-cyanoethoxy) phosphine (343.6 mg, 1.14 mmol, 1.3 eq) and 4, 5-dicyanoimidazole (113.6 g, 0.96 mmol, 1.1 eq) in order at room temperature. The reaction mixture was stirred for 3 hour at room temperature. The reacting solution was diluted with 40 mL of dichloromethane and washed with 2×10 mL of saturated aqueous sodium bicarbonate and 1×10 mL of saturated aqueous sodium chloride respectively. The organic phase was dried over anhydrous sodium sulfate, filtered and concentrated till no residual solvent left under reduced pressure. The residue was purified by Flash-Prep-HPLC. The fractions (1000 mL) were diluted with 1500 mL of dichloromethane. The organic phase was dried over anhydrous sodium sulfate, filtered and concentrated under reduced pressure. 312.4 mg (85% pure, 54%) of 10-12 was obtained as a white solid. MS m/z [M+H]+ (ESI): 772. 1H NMR (300 MHz, DMSO-d₆) δ 11.25 (s, 1H), 7.54-7.26 (m, 11H), 7.25-7.16 (m, 2H), 6.95 (m, 2H), 6.20 (m, 1H), 4.23 (t, J=4.0 Hz, 1H), 4.16-3.94 (m, 1H), 3.83-3.42 (m, 9H), 2.84-2.64 (m, 2H), 1.94 (d, J=14.4 Hz, 1H), 1.76 (m, 4H), 1.65 (d, J=2.6 Hz, 3H), 1.12 (m, 12H). P NMR (300 MHz, DMSO-d₆): δ147.8, 146.86.

### Example 11

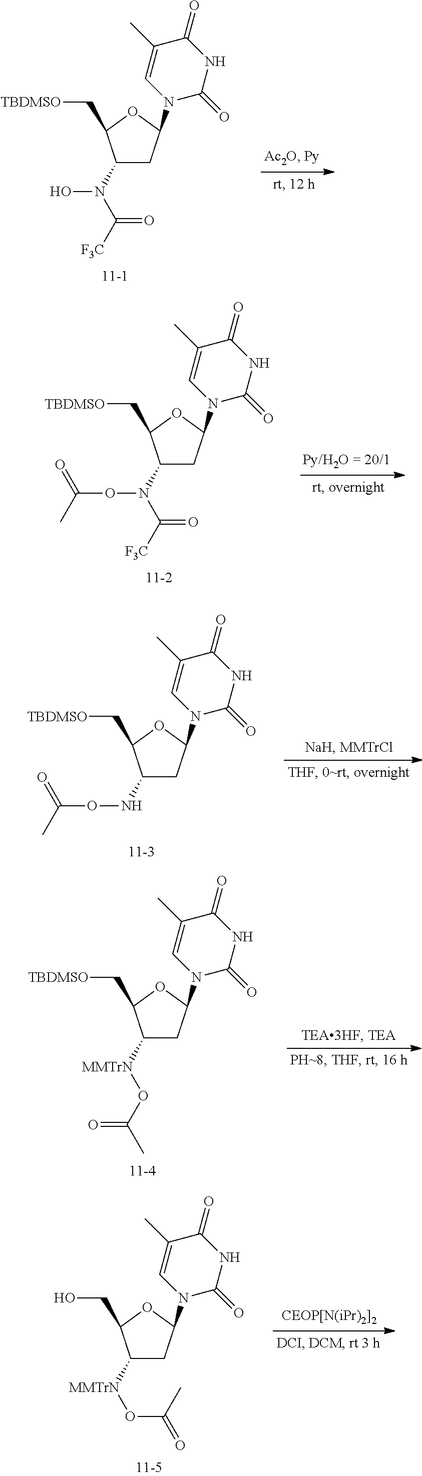

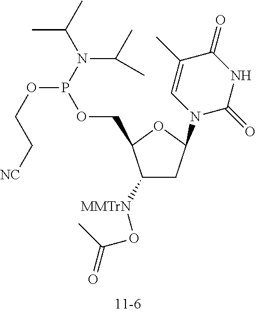

### Synthesis of 11-2

To a solution of 11-1 (28 g, 59.96 mmol, 1.0 equiv) in 280 mL dry Pyridine was added acetic anhydride (9.17 g, 89.94 mmol, 1.5 equiv) in portions at room temperature. The reaction mixture was stirred for 12 h at room temperature. The resulting mixture was concentrated under reduced pressure. The residue was dissolved in 2000 mL of dichloromethane and washed with 2×500 mL of water. The organic phase was dried over anhydrous sodium sulfate, filtered and concentrated under reduced pressure. The crude product was purified by Flash-Prep-HPLC. The fractions (1100 mL) were diluted with 2000 mL of dichloromethane and the organic phase dried over anhydrous sodium sulfate. The solid was filtered out. The filtrate was concentrated under reduced pressure. 23 g (75%) of 11-2 was obtained as a white solid. MS m/z [M+H]+(ESI): 510.

### Synthesis of 11-3

11-2 (23 g, 45.18 mmol) was dissolved in 230 mL of pyridine/water=20/1 at room temperature and stirred overnight at room temperature. The resulting mixture was concentrated under reduced pressure. The crude product was purified by Flash-Prep-HPLC. The fractions (1000 mL) were diluted with 2000 mL of dichloromethane and the organic phase dried over anhydrous sodium sulfate. The solid was filtered out. The filtrate was concentrated under reduced pressure. 14.5 g (83%) of 11-3 was obtained as a light yellow solid. MS m/z [M+H]+ (ESI): 414. 1H NMR (400 MHz, DMSO-d₆) δ 11.35 (s, 1H), 8.06 (d, J=4.9 Hz, 1H), 7.50 (d, J=1.5 Hz, 1H), 6.13 (m, 1H), 3.92 (m, 1H), 3.84 (m, 1H), 3.73 (m, 2H), 2.22 (m, 1H), 2.05 (s, 4H), 1.78 (d, J=1.2 Hz, 3H), 0.89 (s, 9H), 0.08 (d, J=1.7 Hz, 6H).

### Synthesis of 11-4

To a solution of sodium hydride (60%, w/w) (5.6 g, 144.2 mmol, 8.5 equiv) in 56 ml of tetrahydrofuran with an inert atmosphere of argon gas, was added 11-3 (7 g, 16.99 mmol, 1.0 equiv) at 0° C. After 10 min, 4-methoxy-triphenyl methane (11.5 g, 37.38 mmol, 2.2 equiv) was added at 0° C. The resulting solution was stirred overnight at room temperature and diluted with 250 mL of tetrahydrofuran and filtrated. The pH value of the filtrate was adjusted to 7-8 with acetic acid, washed with 2×100 mL of saturated aqueous sodium bicarbonate and 1×100 mL of saturated aqueous sodium chloride respectively. The organic phase was dried over anhydrous sodium sulfate, filtered and concentrated under reduced pressure. The crude product was purified by Flash-Prep-HPLC. The fractions (1000 mL) were diluted with 2000 mL of dichloromethane and the organic phase dried over anhydrous sodium sulfate. The solid was filtered out. The filtrate was concentrated under reduced pressure. 3.5 g (31%) of 11-4 was obtained as a white solid. MS m/z [M+H]+ (ESI): 686.

### Synthesis of 11-5

To a solution of 11-4 (4.2 g, 6.13 mmol, 1.0 equiv) in 40 ml of tetrahydrofuran with an inert atmosphere of nitrogen, was added triethylamine (3.72 g, 36.78 mmol, 6.0 equiv) and triethylamine trihydrofluoride (1.97 g, 12.26 mmol, 2.0 equiv) in order at room temperature. The resulting solution was stirred for 16 h at room temperature, and diluted with 300 mL of dichloromethane, washed with 2×100 mL of saturated aqueous sodium bicarbonate and 1×100 mL of saturated aqueous sodium chloride respectively. The organic phase was dried over anhydrous sodium sulfate, filtered and concentrated under reduced pressure. The residue was purified by Flash-Prep-HPLC. The fractions were diluted with 1000 mL of dichloromethane and the organic phase was dried over anhydrous sodium sulfate. The solid was filtered out. The filtrate was concentrated under reduced pressure. 1.6 g (48%) of 11-5 was obtained as a white solid. MS m/z [M+Na]+ (ESI): 594. 1H NMR (300 MHz, DMSO-d₆) δ 11.22 (s, 1H), 7.57 (s, 1H), 7.39 (s, 10H), 7.21 (s, 2H), 6.97 (s, 2H), 6.18 (t, J=6.7 Hz, 1H), 5.03 (t, J=4.7 Hz, 1H), 4.19 (d, J=15.7 Hz, 2H), 3.77 (s, 3H), 3.63 (d, J=7.7 Hz, 1H), 3.48-3.36 (m, 1H), 2.14-1.98 (m, 1H), 1.67 (m, 7H).

### Synthesis of 11-6

To a solution of 11-5 (500 mg, 0.875 mmol, 1.0 equiv) in 5 mL of dichloromethane with an inert atmosphere of nitrogen, was added bis (diisopropylamino) (2-cyanoethoxy) phosphine (343.6 mg, 1.14 mmol, 1.3 equiv) and 4, 5-dicyanoimidazole (113.6 g, 0.96 mmol, 1.1 equiv) in order at room temperature. The reaction mixture was stirred for 3 hours at room temperature. The reacting solution was diluted with 40 mL of dichloromethane and washed with 2×15 mL of saturated aqueous sodium bicarbonate and 1×15 mL of saturated aqueous sodium chloride respectively. The organic phase was dried over anhydrous sodium sulfate, filtered and concentrated till no residual solvent left under reduced pressure. The residue was purified by Flash-Prep-HPLC. The fractions (800 mL) were diluted with 1500 mL of dichloromethane. The organic phase was dried over anhydrous sodium sulfate, filtered and concentrated under reduced pressure. 301.5 mg (85% pure, 44%) of 11-6 was obtained as a white solid. MS m/z [M+H]+ (ESI): 772. 1H NMR (300 MHz, DMSO-d₆) δ 11.25 (s, 1H), 7.74-7.24 (m, 11H), 7.20 (m, 2H), 6.95 (m, 211), 6.20 (m, 1H), 4.23 (s, 1H), 4.01 (s, 1H), 3.89-3.36 (m, 9H), 2.82-2.64 (m, 2H), 1.96 (s, 1H), 1.75 (d, J 4.0 Hz, 4H), 1.65 (d, J=2.7 Hz, 3H), 1.12 (m, 12H). P-NMR (DMSO, 300 Hz, ppm): 146.8, 147.8.

### Example 12

### Preparation of 2′-Moe-U Nucleoside

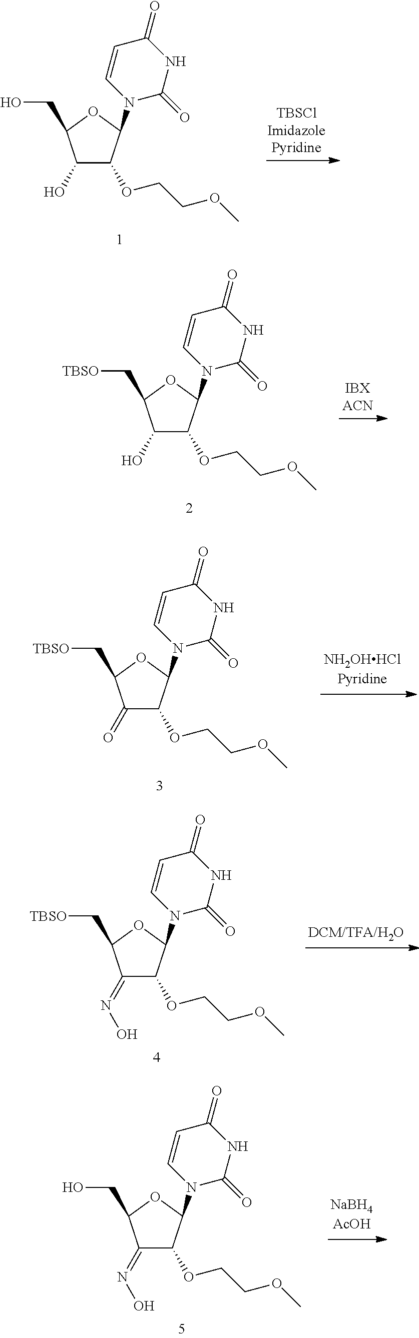 [SMILES: COCCO[C@@H]1[C@H](O)[C@@H](CO)O[C@H]1n1ccc(=O)[nH]c1=O ; COCCO[C@@H]1[C@H](O)[C@@H](CO[Si](C)(C)C(C)(C)C)O[C@H]1n1ccc(=O)[nH]c1=O ; COCCO[C@@H]1C(=O)[C@@H](CO[Si](C)(C)C(C)(C)C)O[C@H]1n1ccc(=O)[nH]c1=O ; COCCO[C@@H]1/C(=N\O)[C@@H](CO[Si](C)(C)C(C)(C)C)O[C@H]1n1ccc(=O)[nH]c1=O ; COCCO[C@@H]1/C(=N\O)[C@@H](CO)O[C@H]1n1ccc(=O)[nH]c1=O]

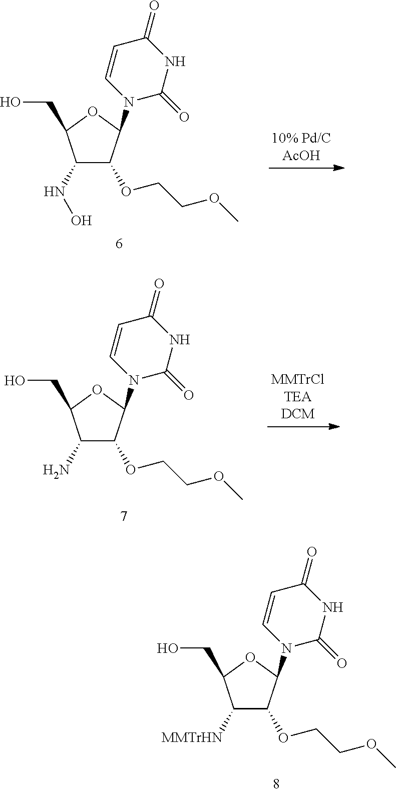

Preparation of (2): To a solution of 1 (10.0 g, 33.1 mmol) in pyridine were added imidazole (4.5 g, 66.2 mmol) and TBSCl (7.5 g, 49.5 mmol). Then the mixture was stirred at r.t. for 15 h. The mixture was diluted with EA and washed with water and brine. The organic layer was dried over Na₂SO₄ and concentrated to give the crude. The crude was purified by silica gel column (PE:EA=5:1˜1:3) to give 2 (12.0 g, 28.8 mmol, 88.0% yield) as a white solid. ESI-LCMS: m/z 417 [M+H]⁺.

Preparation of (3): A solution of 2 (12.0 g, 28.8 mmol) in ACN (120 mL) was added IBX (16.1 g, 57.6 mmol). The mixture was stirred at 80° C. for 5 h. LC-MS showed 2 was consumed completely. The suspension was filtered and the combined filtrate was concentrated to give crude 3 (12.0 g) as a white solid which was used directly for next step. ESI-LCMS: m/z 415 [M+H]⁺.

Preparation of (4): To a solution of crude 3 (12.0 g) in pyridine (100 mL) was added NH₂OH·HCl (3.1 g, 43.2 mmol). The reaction mixture was stirred at r.t. for 1 h. LCMS showed 3 was consumed completely. Water was added and the product was extracted with EA. The combined organic layer was washed with brine and dried over Na₂SO₄. Then the organic was concentrated to give the crude. The crude was purified by silica gel column (PE:EA=5:1˜1:3) to give 4 (10.0 g, 23.3 mol, 80.2% yield over 2 steps) as a white solid. ESI-LCMS: m/z 430 [M+H]⁺.

Preparation of (5): To a solution of 4 (10.0 g, 23.3 mmol) in DCM (100 mL) were added TFA (20 mL) and water (5 mL). The mixture was stirred at r.t. for 15 h. TLC showed 4 was consumed completely. The mixture was concentrated to give the crude. The crude was washed with MTBE to give the crude 5 (5 g) as a black solid which was used directly for the next step. ESI-LCMS: m/z 316 [M+H]⁺.

Preparation of (6): To a solution of 5 (1.0 g, 3.1 mmol) in AcOH (10 mL) was added NaBH4 slowly at 5° C. Then the reaction mixture was stirred at r.t. for 1 h. LCMS showed 6 was consumed completely. The solvent was removed in vacuo. The residue was purified by silica gel column (10% MeOH in DCM) to give 6 (500 mg, 1.5 mmol) as a yellow solid. ¹H-NMR (DMSO-do, 400 MHz): δ ppm 11.42 ((s, 1H), 8.01 (d, J=8.1 Hz, 1H), 7.51 (s, 1H), 5.88 (d, J=4.4 Hz, 1H), 5.62 (d, J=5.2 Hz, 1H), 5.20 (s, 1H), 4.08 (t, J=5.2 Hz, 1H), 3.95-3.94 (m, 1H), 3.74-3.63 (m, 3H), 3.63-3.45 (m, 4H), 3.24 (s, 3H). ESI-LCMS: m/z 318 [M+H]⁺.

Preparation of (7): To a solution of 6 (300 mg, 0.9 mmol) in aqueous AcOH (90%, 10 mL) was added Pd/C (10%, 50 mg). Then the mixture was stirred under atmosphere pressure of H₂ at r.t. for 3 h. The catalyst was filtered through Celite and the filtrate was concentrated in vacuo. The residue was purified by silica gel column (10% MeOH in DCM) to give 7 (180 mg. 0.6 mmol) as a yellow solid. ESI-LCMS: m/z 302 [M+H]⁺;

Preparation of (8): To a solution of 7 (180 mg, 0.6 mmol) in DCM (5 mL) was TEA (181 mg, 1.8 mmol). Then MMTrCl (184 mg, 0.6 mmol) was added to the reaction mixture. The reaction mixture was stirred at r.t. for 1 h. TLC showed 7 was consumed. Water was added and the product was extracted with DCM. The combined organic layer was washed with brine and dried over Na₂SO₄. Then the organic was concentrated to give the crude. The crude was purified by silica gel column (PE:EA=5:1˜1:2) to give 8 (250 mg, 0.43 mmol, 71.6% yield) as a white solid. ESI-LCMS: m/z 572 [M−H]⁻; ¹H-NMR (DMSO-d₆, 400 MHz): δ ppm 11.26 (s, 1H), 7.95 (d, J=8.4 Hz, 1H), 7.47-7.44 (m, 4H), 7.34-7.17 (m, 8H), 6.82 (d, J=8.8 Hz, 2H), 5.50-5.48 (m, 2H), 5.13 (t, J=3.6 Hz, 1H), 4.05-3.98 (m, 3H), 3.78 (s, 3H), 3.52-3.49 (m, 1H), 3.34-3.32 (m, 2H), 3.14 (s, 3H), 3.08-3.04 (m, 1H), 2.89-2.86 (m, 1H), 2.70 (d, J=10.0 Hz, 1H), 1.51 (d, J=4.4 Hz, 1H).

### EQUIVALENTS

The present disclosure is not to be limited in terms of the particular embodiments described in this application, which are intended as single illustrations of individual aspects of the present disclosure. Many modifications and variations of this present disclosure can be made without departing from its spirit and scope, as will be apparent to those skilled in the art. Functionally equivalent methods and apparatuses within the scope of the present disclosure, in addition to those enumerated herein, will be apparent to those skilled in the art from the foregoing descriptions. Such modifications and variations are intended to fall within the scope of the present disclosure. It is to be understood that this present disclosure is not limited to particular methods, reagents, compounds compositions, or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 items refers to groups having 1, 2, or 3 items. Similarly, a group having 1-5 items refers to groups having 1, 2, 3, 4, or 5 items, and so forth.

All patents, patent applications, provisional applications, and publications referred to or cited herein are incorporated by reference in their entirety, including all figures and tables, to the extent they are not inconsistent with the explicit teachings of this specification.

What is claimed is:

1. A method of producing a nucleoside of formula (III): 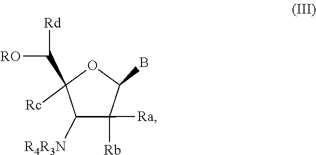
wherein
wherein B is an optionally protected or modified nucleobase;
R is H, a counterion or a protecting group, PG1;
Ra and Rb are each independently selected from the group consisting of H, halogen, R¹, OR¹, OPG1 and OR²OR¹;
Rc is selected from the group consisting of H, R¹, OPG2, OR¹ and N(R₉)₂;
Rd is selected from the group consisting of H and R¹;
R₃ is PG2 or OPG3; and
R₄ is H, OAc, or Ac, or
R₃ and R₄ together form a cyclic protecting group, cPG;
R¹ is C₁₋₃alkyl optionally substituted with one or more halogen or PG;
R² is C₁₋₅alkylene optionally substituted with one or more halogen; and
each R₉ is independently selected from the group consisting of H and C₁₋₆alkyl, comprising the steps of:
providing a 3′-oxime modified nucleoside,
converting the 3′-oxime modified nucleoside to a 3′-NH modified nucleoside, and
converting the 3′-NH modified nucleoside to a compound of formula (III).

2. The method of claim 1, wherein R¹ is a fluorine.

3. The method of claim 1, wherein R² is a fluorine

4. The method of claim 1, wherein Rb is selected from OCF₂—CH₃, OCH₂CH₂OMe, OMe, OEt, OCH₂F, F, OTBDMS.

5. The method of claim 1, wherein Rb is selected from OCF₂—CH₃, OCH₂CH₂OMe, OMe, OEt, OCH₂F, F, OTBDMS.

6. The method of claim 1, wherein the 3′-oxime modified nucleoside is represented by the following formula (I): 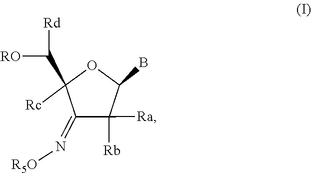
wherein
B, R, Ra, Rb, Rc, Rd, P, R₁, R₂ are the same as formula (III) and
R₅ is H or a C₁₋₆alkyl group, optionally substituted with an aryl group.

7. The method of claim 1, wherein the 3′-NH modified nucleoside is represented by the following formula: 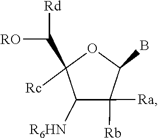
wherein
B, R, Ra, Rb, Rc, Rd, P, R₁, R₂ are the same as formula (I) and
R₆ is a C₁₋₃alkyl or a protecting group.

8. The method of claim 1, wherein the 3′-oxime modified nucleoside is converted to 3′-NH modified nucleoside directly through a hydroxylamine intermediate compound.

9. The method of claim 1, wherein the 3′-oxime modified nucleoside is converted to 3′-NH modified nucleoside through a hydroxylamine intermediate compound in two or less steps.

10. The method of claim 1, wherein converting the 3′-oxime modified nucleoside to a 3′-NH modified nucleoside comprises a selective reduction of the 3′-oxime moiety.

11. The method of claim 10, wherein the selective reduction comprises use of NaB(OAc)₃ or pinacolborane.

12. The method of claim 1, wherein B is a protected or unprotected purine or pyrimidine.

13. The method of claim 1, wherein B is a protected or unprotected adenosine.

14. The method of claim 1, wherein B is a protected or unprotected guanosine.

15. The method of claim 1, wherein B is a protected or unprotected uridine.

16. The method of claim 1, wherein B is a protected or unprotected cytidine.

17. The method of claim 1, wherein the method does not include a chromatography purification step.

18. The method of claim 1, wherein the method is conducted on 1 kg or more 3′-oxime modified nucleoside.

19. The method of any one of claims 1-12, wherein B is a protected or unprotected adenosine and Rb is F or MOE.

20. The method of claim 19, wherein adenosine is not protected with Bz.

21. The method of any one of claims 1-12, wherein B is a protected or unprotected guanosine and Rb is F or MOE.

22. The method of any one of claims 1-21, further comprising preparing an oligonucleotide using the compound of formula (III).

23. A compound represented by formula (I′) or (II′): 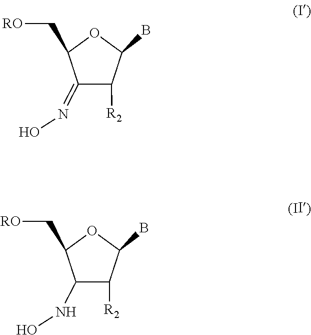
wherein
B is an optionally protected nucleobase,
R is H, —OH, a counterion, or a protecting group,
R₂ is F, OR₇ or OR₈OR₇,
R₇ is a C₁₋₃alkyl or fluoroalkyl, and
R₈ is a C₁₋₅alkylene or fluoroalkylene.

24. The compound of claim 23, wherein R₂ is selected from OCF₂—CH₃, OCH₂CH₂OMe, OMe, OEt, OCH₂F, F, OTBDMS.

25. The compound of claim 23, wherein B is a protected or modified nucleobase.

26. The compound of claim 23, wherein B is a protected or modified adenine, guanine, cytosine, uridine or thymine.

27. The compound of claim 23, wherein B is a protected or modified purine or pyrimidine.

28. The compound of claim 23, wherein B is a protected adenine.

29. A method of producing a nucleoside of formula (III-A): 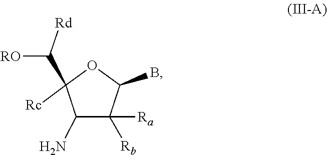
wherein
wherein B is an optionally protected nucleobase;
R is H, a counterion or a protecting group, PG1;
Ra and Rb are each independently selected from the group consisting of H, halogen, R¹, OR¹, OPG1 and OR₂OR¹;
Rc is selected from the group consisting of H, R¹, OPG2, OR¹ and N(R₉)₂;
Rd is selected from the group consisting of H and R¹;
R¹ is C₁₋₃alkyl optionally substituted with one or more halogen or PG;
R₂ is C₁₋₅alkylene optionally substituted with one or more halogen; and
each R₉ is independently selected from the group consisting of H and C₁₋₆alkyl, comprising the steps of:
providing a 3′-oxime modified nucleoside,
converting the 3′-oxime modified nucleoside to compound of formula (III-A).

30. The method of claim 29, further comprising protecting the amine at the 3′ position of the compound of formula (III-A).

31. A method of producing an antisense oligonucleotide (ASO) or a small interfering RNAs (siRNA), the method comprising preparing the compound of formula (III) using the method of any one of claims 1-21.
